(19) United States
(12) Patent Application Publication
(10) Pub. No.: US 2021/0041441 A1
(43) Pub. Date: Feb. 11, 2021

(54) DISCOVERY OF NOVEL MOLECULES AND REPURPOSED DRUGS FOR RAS FAMILY GTPases

(71) Applicants: UNM Rainforest Innovations, Albuquerque, NM (US); The University of North Carolina at Chapel Hill, Chapel Hill, NC (US)

(72) Inventors: Larry A. Sklar, Albuquerque, NM (US); Tudor I. Oprea, Albuquerque, NM (US); Anna Waller, Albuquerque, NM (US); Angela Wandinger-Ness, Albuquerque, NM (US); Mark K. Haynes, Albuquerque, NM (US); Sharon Campbell, Chapel Hill, NC (US); Harold A. Ames, Albuquerque, NM (US)

(21) Appl. No.: 16/978,364

(22) PCT Filed: Mar. 8, 2019
(86) PCT No.: PCT/US2019/021301

§ 371 Date: Sep. 4, 2020

Related U.S. Application Data
(60) Provisional application No. 62/689,512, filed on Jun. 25, 2018.

(51) Int. Cl. G01N 33/574; A61K 31/407; A61K 31/155

(52) U.S. Cl. CPC G01N 33/5748; G01N 2500/02; A61K 31/155; A61K 31/407

(57) ABSTRACT

The present invention is directed to compounds, compositions and methods for modulating RAS family GTPases, in particular KRas, HRas and NRas GTPases. These GTPases are upregulated in cancer and in other tissue and represent appropriate targets for therapy. Methods for identifying the activity of compounds with respect to these and other GTPases in multiplex flow cytometry systems represents another aspect of this invention.

### RELATED APPLICATIONS

This application claims the benefit of priority of provisional applications Ser. Nos. US62/640,162, filed Mar. 8, 2018 and US62/689,512, filed Jun. 25, 2018, each of said applications being incorporated by reference in their entirety herein.

This invention was made with government support under Grant Nos. P50 GM085273 and TR0001111 awarded by National Institutes of Health. The government has certain rights in the invention.

### FIELD OF THE INVENTION

The present invention is directed to compounds, compositions and methods for modulating RAS family GTPases, in particular, KRas, HRas and NRas GTPases and treatment of disease which is mediated through these GTPases or where these GTPases play a role in a disease state and/or condition. The present invention is also directed to technology for discovery of compounds, compositions, and methods for identifying compounds/compositions which modulate RAS family GTPases, in particular KRas, HRas and NRas GTPases. These GTPases are upregulated in cancer and in other tissue and represent appropriate targets for therapy with the compounds which are identified here.

### BACKGROUND AND OVERVIEW OF THE INVENTION

As RAS genes comprise the most frequently mutated gene family in human cancer, the validated role of mutationally activated RAS genes in driving cancer development and growth has stimulated comprehensive efforts to develop therapeutic strategies to block mutant Ras function for cancer treatment. Despite more than three decades of intensive effort, no effective Ras-targeted therapies have reached the clinic while kinases have yielded dozens of approved drugs. The inventors challenge the currently held perception that all RAS mutations are “created equal” and argue that pursuit of a pan-Ras therapeutic approach will not be successful. Instead, we suggest that the recent discovery of a therapeutic approach targeting one RAS mutation (G12C) establishes the premise that screening specific Ras mutant proteins will reveal mutation- and cancer type-specific vulnerabilities for mutation-selective anti-Ras therapies. Additionally, the recent identification of unique pockets and protein-protein interaction interfaces dictate unique behaviors of individual Ras proteins (HRas, KRas and NRas) further supporting the premise that Ras selective compounds will have significant utility. We used our unique multiplexed experimental approach that ensures the stability of Ras and Ras-related GTPase and allows comparative assessment of target sensitivity during screening with compound libraries. The approach has demonstrated utility for detection of hits and development of robust leads that are active against select or multiple GTPases. Through combined testing of off-patent drugs, cheminformatics to identify the most promising scaffolds, preclinical and clinical testing, two enantio-selective scaffolds derived from off-patent drug libraries were shown to have clinical translational utility. The publication and patent track records of the present inventors suggest that Ras GTPases are targets useful in the treatment of disease states and/or conditions which are modulated through Ras GTPases.

Ras proteins are molecular switches that regulate cellular metabolism and growth, toggle between inactive ‘off’ and active ‘on’ states through a process highly regulated by cellular factors¹⁻³. Mutations in RAS commonly found in human cancer cause Ras proteins to chronically switch on, resulting in deregulated growth control⁴. Approximately one-third of all human cancers contain activating mutations in RAS genes that drive cancer development and growth^{5,6}. Still, the specific roles of particular RAS mutations in oncogenesis are poorly understood⁷. Additionally, information on the complex regulation through conformation, oligomerization, and membrane orientation/organization of individual Ras proteins is still emerging⁸⁻¹². Several small molecule and protein inhibitors of Ras have been identified through in silico, high content imaging, crystallographic, and NMR-based strategies¹³⁻²⁰. However, despite the identification of compounds that block mutant Ras protein function, moving effective Ras-targeted therapies into the clinic remains an unmet goal^{5,7,21-23}. Thus, further characterization and discovery is needed for a more knowledgeable approach to anti-Ras therapy.

Numerous drugs have been identified and developed to modulate kinases; thus part of the motivation of the present inventors was to identify drugs and other chemical entities that could be found to modulate GTPases. Like kinases, GTPases act as stimulus sensors and utilize nucleotide binding and hydrolysis to govern conformational changes, membrane organization, and protein-protein interactions¹⁻⁴. Both kinases and GTPases constitute large protein families which were at one time or another dubbed ‘undruggable’ due to the idea that conserved substrate binding pockets make it impossible to develop selective or specific drugs. Kinase targeted drugs are now a notable success story^{24,25}. The present inventors thus hypothesize that GTPases are high value, individually druggable targets, which like kinases are a large family of enzymes. Unlike kinases today, there are few inhibitors (FTI inhibitors, zoledronic acid) for GTPases that have reached the clinic and the noted examples affect multiple targets causing adverse effects. With over 500 kinases encoded in the human genome and large numbers of human diseases caused by kinase dysregulation, kinases have a history as targets for therapeutic intervention^{26,27}. After the serendipitous discovery of staurosporine²⁸, high throughput screening identified more ATP-competitive kinase inhibitors, with optimization leading to trials and approval. Structural analyses of kinases with inhibitors bound enabled kinase drug discovery to employ structure-based rational design, using lead optimization and fragment-based strategies. Notably, compound libraries generated by combinatorial chemical synthesis have facilitated the discovery of new kinase inhibitors where the library members can be individual compounds or compound mixtures²⁹. This history of therapeutic targeting of kinases offers relevant perspectives for targeting GTPases that can now be exploited.

Progress into clinical trials for drugs targeting GTPases has been slow, potentially due to several factors. First, the nucleotide binding domain is relatively small and GTPases assume a relatively smooth and globular structure^{4,30}, making it more difficult to predict drug binding pockets. Second, the binding affinity of the guanine nucleotide towards GTPases is high suggesting a problem for competition^{31,32}. Third, the activity of GTPases is regulated by separate proteins like GEF and GAP proteins³³. Finally, GTPases play diverse roles in cell physiology ranging from cytoskeletal changes to protein translation which suggests that toxicity from unwanted side effects could be severe, especially for compounds that are not selective or specific. Still, there has been progress. For example, virtual screening identified Rho and Rac inhibitors that block the interactions between the GTPase and its effectors^{14,37}, and in silico docking has identified inhibitors of Ras and its downstream effector proteins^{17,19,38-49}. Automated and efficient screening methods now include our multiplexing strategies⁴¹. Flow cytometry based multiplex screening assay⁴² allows individual GTPases to be linked to microsphere bead sets with distinct fluorescence intensities in the red fluorescence channel. The extent of fluorescent GTP binding to individual GTPases in the presence of test compounds can then be analyzed in another fluorescent channel. This method allows the potency and selectivity of a compound towards several GTPases to be revealed simultaneously and reduces quantities of GTPases compared to plate-based homogeneous assays. Also, the use of GST-GTPase chimeras and their immobilization on beads stabilizes GTPases against denaturation and may mimic oligomeric status. The inventors have been involved with previously conducted: a) high throughput screening of multiple GTPases against ˜200,000 compounds from the Molecular Libraries Small Molecule Repository to identify regulators of nucleotide binding^{35,36,42}; b) quantitative analyses of cellular GTPase activities using small volume samples⁴³; and c) small molecule mechanism of action studies using real-time kinetic measurements of ligand or effector binding,^{44,45}. Our screening and multi-tiered analysis platforms have identified both competitive and allosteric, selective inhibitors of Rho-family GTPases with clinical applicability^{46,47}. We also identified small molecules that potentiate GTP binding⁴⁸.

The overall premise which led to the present invention is that different KRAS mutations drive cancer by distinct mechanisms and hence require distinct therapeutic strategies. Recent identification of small molecules that allosterically and covalently inhibit the KRas G12C mutant frequently found in lung cancer support this hypothesis⁴⁰⁻⁵¹. Thus, we have adapted our multiplex screening technology^{42,52-55} and have performed proof-of-principle screens of small chemical libraries using wild-type KRas and its prevalent point mutant proteins (G12A, G12D, G12V, G12C, G13D, Q61R, Q61L, and Q61H). Initial experience with this screen supports the idea that we can identify Ras selective small molecules and that there is merit in identifying molecules that bind to and regulate nucleotide binding to codon-specific KRAS mutations found with high frequency in human cancers. Identified active compounds and prevalent scaffolds can be used to confirm chemical vulnerabilities of Ras family proteins

### BRIEF DESCRIPTION OF THE INVENTION

In one embodiment, the present invention is directed to discovery of selective compounds which modulate human RAS GTPases, in particular KRAS, NRAS and HRAS and methods of treating disease states and/or conditions which are modulated through human RAS GTPases. These disease states and/or conditions include immune dysfunction, pigmentation or neurological disorders which occur as a consequence of impaired GTPase function and/or functional insufficiency. Additional disease states and/or conditions which may be favorably influenced by treatment with the present compounds include cancers (e.g., leukemias, colorectal cancer, pancreatic cancer, lung cancer, ovarian cancer, lung adenocarcinoma, mucinous adenoma, ductal carcinoma of the pancreas, colorectal cancer, among others, often associated with KRAS, thyroid cancer, salivary duct carcinoma, epithelial-myoepithelial carcinoma, kidney cancer, astrocytoma, among others, often associated with HRAS and melanoma, often associated with NRAS), histiocyte disorders (e.g. Rosai-Dorfman disease/sinus histiocytosis with massive lymphadenopathy), Noonan syndrome (NS), Noonan syndrome with multiple lentigines, Leopard syndrome, cardiofacio-cutaneous syndrome, neurofibromatosis type I (NF1) and secondary effects of same including fibromas, scoliosis, long bone dysplasmia, osteoporosis and cognitive impairment, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 and hypertrophic cardiomyopathy (HCM), among others. The method comprises administering an effective amount of a compound identified herein to a patient in need, optionally in combination with at least one additional bioactive agent, and further optionally, at least one pharmaceutically acceptable carrier, additive or excipient. In embodiments, the present invention is directed to the treatment of cancer. In certain embodiments, the compound identified herein is combined with at least one additional bioactive agent in the treatment of a disease state and/or condition. In certain embodiments, the additional bioactive agent is at least one additional anticancer agent. In other embodiments, the additional bioactive agent is an agent which is separately useful for the treatment of a disease and/or a condition, often the same disease state or condition or a related disease state or condition for which the Ras modulator may be administered.

In other embodiments, the present invention is directed to the technology and methods used to identify pharmaceutical compositions comprising an effective amount of a compound identified herein, optionally in combination with an effective amount of an additional bioactive agent (often an additional anticancer agent or other agent useful in the treatment of cancer), in combination with a pharmaceutically acceptable carrier, additive or excipient. In embodiments, the compound is an antagonist or inhibitor of KRas, NRas or HRas GTPase. In other embodiments, the compound is an agonist of KRas, NRas or HRas GTPase. The Ras GTPase may be a wild type protein of a mutant protein as described herein.

Compounds according to the present invention are discovered employing the technology embodied in this claim that are described as specific inhibitors or agonists of Ras GTPases (ie., KRas, NRas and HRas GTPases), pan-inhibitors or pan-activators of Ras GTPases (ie., the inhibitor or activator is active across a number of GTPases, including mutant GTPases), mixed activity modulators (i.e., within a family of GTPases the type of activity is the same, but outside of the family, the activity may vary such that an inhibitor may become an agonist) or other potential modulators (i.e., within the same family of GTPases such as KRas, NRas or HRas the compound exhibits categorically different activity as an inhibitor or agonist).

Thus, the Ras and Ras-related GTPases are important targets for the development of small molecule agonists well as antagonists for therapy of certain disease states and/or conditions, to aid studies of disease mechanism or to serve as scaffolds or pharmacophores for future therapeutics. The present invention identifies technology for discovery of modulators of Ras GTPases as set forth in the present application and in the examples A, B, C, D, E) which provide methods and results for 1) optimization of buffers for stability and display of KRas WT and mutants; 2) screening of multiplex KRas proteins; 3) dose-response of active compounds; 4) selectivity of active compounds; 5) mechanism of action of a representative active compound.

The small molecules of the present invention include antagonists, activators (agonists), including specific (for individual proteins, including mutant versions of such proteins). Importantly, such specific- and pan-GTPase modulators, including inhibitors and activators could provide advantages over genetic methodologies in cell-based assays, for measuring initial and/or acute response of reversibly altering activities of GTPases. Furthermore, these molecules provide a scaffold for structure-based design of agonists and antagonists against Rho-family GTPases to complement existing antagonists or inhibitors. As Ras superfamily GTPases gain increasing traction as viable targets for further probe and drug discovery, the present invention provides a chemical platform for the rationale design of selective activators of key Ras superfamily members that could represent a boon for expanded understanding of the biology and pharmacology of small GTPases and therapy of disease states and/or conditions which are modulated through these proteins.

The following compounds among others were identified using the technology for compound discovery as presented in the attached examples section.

Pan Activators of Ras Family GTP Binding

Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid and Iopanic acid have been found to be pan activators of Ras family GTP binding. These compounds exhibited an increase in Bodipy-GTP binding in the presence of compound.

Pan Inhibitors of Ras Family GTP Binding

Istradefylline, PR-619, N6022, Diffractaic acid, IPA 3, Fisetin, Folic acid, GSK 3787 have been found to be pan inhibitors of Ras family GTP binding. These compounds exhibited a decrease in Bodipy-GTP binding in the presence of compound.

Selective Inhibitors of GTP Binding to RAS Proteins, but Not Q61 KRAS Mutants

Guanabenz acetate, Chlorprothixene hydrochloride, NSC 663284, Trifluperazine Hydrochloride have been found to be selective inhibitors of GTP binding to Ras family proteins (these compounds exhibited a decrease in Bodipy-GTP binding in the presence of compound), but not to Q61 KRas mutants.

Mixed Activity Modulators of GTP Binding

Ipsapirone, GF109203X (selective activator non-RAS), Beta Lapachone (RAS activator/non-RAS inhibitor), SF1670 (RAS activator, non-RAS inhibitor), Darapladib (SB480848), PD 198306, Pimethixene Maleate, Oxyquinoline Hemisulfate have been found to be mixed activity modulators of GTP binding.

In embodiments, the present invention is directed to compositions comprising an effective amount of a Ras Family GTPase modulator as described herein in combination with a pharmaceutically acceptable carrier, additive or excipient and further in combination with an effective amount of at least one additional bioactive agent, often an additional anticancer agent.

In embodiments, the present invention is directed to methods of modulating a Ras Family GTPase comprising exposing said GTPase to a compound disclosed herein in effective amounts as an inhibitor or agonist of said GTPase. In embodiments, the Ras GTPase is KRas WT or a mutant, for example, KRas G12v, KRas G13d, KRas G12a, KRas G12c, KRas G12d, KRas Q61H, KRas Q61L, KRas HRas WT or a mutant such as HRas G12v. In preferred aspects the Ras Family GTPase modulator compound is selected from the group consisting of Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid, Iopanic acid, Istradefylline, PR-619, N6022, Diffractaic acid, IPA 3, Fisetin, Folic acid, GSK 3787, Guanabenz acetate, Chlorprothixene hydrochloride, NSC 663284, Trifluperazine Hydrochloride, Ipsapirone, GF109203X (selective activator non-RAS), Beta Lapachone (RAS activator/non-RAS inhibitor), SF1670 (RAS activator, non-RAS inhibitor), Darapladib (SB480848), PD 198306, Pimethixene Maleate, Oxyquinoline Hemisulfate, or a pharmaceutically acceptable salt, stereoisomer, alternative salt or mixture thereof. In embodiments, the GTPase which is modulated (inhibited or activated/up-regulated) is CDC42 WT or a mutant such as CDC42 L61, Rac1 WT or a mutant such as Rac1 L61 and the compounds modulate one or more of the identified GTPases.

In embodiments, the present invention is directed to methods of inhibiting a Ras Family GTPase the method comprising exposing said Ras Family GTPase to an effective amount of at least one compound identified herein as an inhibitor of GTP binding to one or more Ras proteins. In embodiments, the invention is directed to methods of up-regulating or enhancing the activity (through agonist activity) of a Ras Family GTPase, including a mutant, the method comprising exposing said Ras Family GTPase to an effective amount of an agonist of GTP binding to one or more Ras proteins. In embodiments, the Ras Family GTPase is KRas WT or a mutant, such as KRas G12v, KRas G13d, KRas G12a, KRas G12c, KRas G12d, KRas Q61H, KRas Q61L, KRas Q61R, HRas WT or a mutant such as HRas G12v. In embodiments, the GTPase which is inhibited is CDC42 WT or a mutant such as CDC42 L61, Rac1 WT or a mutant such as Rac1 L61 and the compounds modulate one or more of the identified GTPases.

In embodiments, the present invention is directed to methods of treating a disease state or condition which is mediated through a Ras family GTPase, the method comprising administering to a patient or subject in need thereof an effective amount of at least one modulator of GTP binding to one or more RAS proteins, optionally in combination with an effective amount of at least one additional bioactive agent. Disease states or conditions which may be treated pursuant to the present invention include histiocyte disorders (e.g. Rosai-Dorfman disease/sinus histiocytosis with massive lymphadenopathy), Noonan syndrome (NS), Noonan syndrome with multiple lentigines, Leopard syndrome, cardiofacio-cutaneous syndrome, neurofibromatosis type I (NF1) and secondary effects of same including fibromas, scoliosis, long bone dysplasmia, osteoporosis and cognitive impairment, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 and hypertrophic cardiomyopathy (HCM), cancer, other sporadic or genetic diseases or conditions and infections, including those caused by Entamoeba histolytica, among others.

In yet another embodiment the present invention is directed to a method of identifying a compound as a potential selective agonist, antagonist, or regulator of a protein in a flow cytometer comprising:
i. providing in a flow cytometer a multiplex of derivatized flow cytometer beads wherein each said derivatized bead is bound to a GST fusion protein (e.g. GST-GTPase fusion protein) comprising a fused protein and a fluorescently labeled binding partner of said fused protein bound thereto, wherein the binding partner emits fluorescent light upon excitation;
ii. exposing the flow cytometer bead multiplex from step i to a solution comprising at least one compound and preferably a library of unknown activity; and
iii. identifying a compound of said solution as a potential agonist, antagonist, or regulator of said fused GTPase protein within said multiplex if said compound displaces or impacts the binding of said fluorescently labeled binding partner as evidenced by a reduction or increase in the fluorescent light being emitted.

In another embodiment, the present invention is directed to a method of identifying a compound as a potential selective agonist, antagonist, or regulator of a protein in a flow cytometer comprising:
a. providing in a flow cytometer derivatized flow cytometer beads contained within a multiplex wherein the derivatized beads are each bound to a GST fusion protein comprising GST and a fused protein (preferably, a GST-GTPase fusion protein);
b. exposing the flow cytometer bead multiplex from step a to a solution comprising at least one fluorescently labeled compound having the potential for binding to said fused protein; and
c. identifying a compound of said solution as a potential binding partner of said fused protein if said compound binds to said fused protein as evidenced by an increase in the fluorescent light being emitted from said fused protein.

In yet another embodiment, the present invention is directed to a method of identifying a compound or portion of a compound as a binding partner of a protein in a flow cytometer comprising:
a. providing in a flow cytometer a population of derivatized flow cytometer beads wherein each of said derivatized beads is bound to a GST fusion protein comprising GST and a fused protein (preferably GST-GTPase fusion protein) which is fluorescently labeled;
b. exposing the flow cytometer beads from step a to a solution comprising at least one compound having the potential for binding to said fused protein;
c. identifying the compound or a region of the compound as a potential binding partner of the fused protein if the compound binds to the fused protein as evidenced by a decrease in the fluorescent light being emitted from the fused protein;
d. determining the selectivity of said compound identified in step c with respect to individual GTPases by exposing a multiplex of individual fluorescent flow cytometer beads comprising individual GTPases to a solution comprising the compound identified in step c and comparing the binding of the compound with the individual GTPases on the individual fluorescent flow cytometer beads; and
e. determining the selectivity of the compound identified in step c with respect to individual KRas mutants by exposing a multiplex of fluorescent flow cytometer beads comprising individual KRas mutant GTPases to a solution comprising the compound identified in step c and comparing the binding of the compound with the individual KRas mutant GTPases on the fluorescent flow cytometer beads, wherein the selectivity of said compound with respect to KRas mutants and other GTPases is determined by comparing the activities of said compound on said multiplexes comprising both KRas mutant and Ras GTPases with a standard.

In embodiments, the method employs a standard which is used to assess the activity of the compound in the assay compared with the standard.

In embodiments, the method employs a solution comprising a library of compounds, preferably a library of organic small molecules. In embodiments, the compound is identified as a potential agonist, antagonist, or regulator of the fused protein and is subjected to a second assay to determine the activity of said compound as an agonist, antagonist, or regulator of fused proteins within the multiplex.

In embodiments, the fused protein comprises a KRas GTPase, preferably a wild-type or a mutant KRAS (G12D, G12A, G12V, G12C, G13D, Q61R, Q61L, and Q61H).

In embodiments, the used proteins in the second assay utilizes fused proteins comprising one or more GTPase such as a Rab family (˜70 mammalian GTPases), Rho family GTPase, including Rac (e.g. Rac1, Rac2, Rac3) and Cdc42, Ran, or Ras family GTPases.

In embodiments, the fused protein is a GST-GTPase fused protein.

In embodiments, the binding partner is GTP.

In embodiments, the GTPase is a mutant KRas GTPase.

In embodiments, the compound is a protein or polynucleotide.

In embodiments, the compound is a small molecule.

In embodiments, the method utilizes a solution which comprises a series of fluorescently labeled polypeptides or polynucleotides of varying lengths and sequences obtained from a protein or polynucleotide known to be a binding partner of the fused protein.

In embodiments, the protein or polynucleotide compound which binds to said fused protein is further identified by sequencing.

In embodiments the fused protein requires the presence of another molecule in order for the binding partner to bind to the fused protein. In embodiments, the molecule required for the binding partner to bind is fluorescently labeled.

In embodiments, the method is conducted in a flow cytometer which is a high throughput flow cytometer. In embodiments the method is conducted using multiplex high throughput flow cytometry.

### BRIEF DESCRIPTION OF THE FIGURES

FIG. 1 shows multiplex dose dependent Bodipy-GTP binding curves. (A) Dose dependent bodipy-GTP binding curves of KRas proteins (Wild type, G12v, G13d, G12a, G12c, G12d, Q61L, and Q61R) in multiplex. Binding reactions were performed as described in the examples section of the present application. Data shown represent specific binding which is derived by subtracting non-specific binding determined in the presence of excess unlabeled GTP, K_{d} values for Bodipy Fl-GTP binding in this experiment range from 2.3 nM to 4.6 μM. (B) Dose dependent Bodipy-GTP binding curves of non-KRas proteins (HRas WT, HRAS G12v, CDC42WT, CDC42 L61, Rac1 WT and Rac1 L61) in multiplex. K_{d} of the GTP binding in this experiment range from 12.5 to 5.3 nM.

FIG. 2 shows an analysis of kinetic experiments for both A) Protein KRas G12v and B) KRas Q61R. Kinetic binding reactions were analyzed for 42 minutes. Association equilibrium reaction was initiated by the addition of Bodipy-GTP (1 nM) and followed for 15 minutes. Dissociation equilibrium reaction was then initiated by the addition of excess GTP (30 μM) and followed for 25 minutes.

FIG. 3, Table 1, shows singleplex and multiplex analyses of individual, GST-KRas proteins coupled to glutathione-beads evaluated for Bodipy-GTP binding after incubation for 1 hour at 4° C. Binding affinities (EC50) for the KRas proteins were derived using Prism software. Differences between the 2 protocols are within the error of the measurements. Kd values are the average of 4-6 separate experiments.

FIG. 4, Table 2 shows library screening statistics for screens which were conducted in the examples section of the present application.

FIG. 5, Table 3, shows numerous compounds identified pursuant to the present invention and their activities against various RAS protein GTPases. The compounds were classified as PAN Activators, Selective Activators, Pan Inhibitors, Selective inhibitors or Mixed Modulators.

FIG. 6 shows the normalized dose response of activators, inhibitors and mixed modulators of RAS protein GTPases by measuring the binding of Bodipy GTP to multiplex arrays of small GTPases.

FIG. 7 shows (A) the chemical structure of Guanabenz acetate. (B) shows normalized dose response of Guanabenz acetate from 0.1 μM to 100 μM with KRas proteins (WT, G12v, G13d, G12a, G12c, G12d, Q61H, Q61L and Q61R) in multiplex with Bodipy-GTP at 10 nm. (C) shows normalized dose response of Guanabenz acetate from 0.1 μM to 100 μM with non-KRas proteins (HRas WT, HRas G12v, CDC42 WT, CDC42 L61, Rac1 WT and Rac1 L61) in multiplex with Bodipy-GTP at 10 nm.

FIG. 8 shows Tables 4a-4d and Cmax and KI comparison for compounds according to the present invention. The Cmax row provides the maximum concentration of the compound in blood serum. For each compound the KI values were calculated for each protein tested with that compound. Tables 4a and 4b provide all the KI values calculated while Tables 4c and 4 only show the values where the KI has a lower value than the Cmax. ND stands for not determined. “˜” at the beginning of the number signifies that the calculated number was ambiguous.

FIG. 9 shows that RAS genes encode proteins of 189 amino acids containing a highly conserved guanine nucleotide binding domain (G domain) and a hypervariable carboxyl terminal region. RAS is frequently mutated in human cancer, with most point mutations occurring at positions 12, 13 and 61 in the G-domain.

FIG. 10 shows assays for multiplex screening, and follow-up measurements of compound mechanism of action on nucleotide binding or effector protein interactions are in hand and are part of the work-flow. Shown are results for GTPase inhibitor (CID1067700) that acts as a competitive inhibitor of nucleotide binding and prevents adoption of active conformation in vitro and in cells (8, 11, 13-14).

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated to describe an amount of compound, composition or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers, individual optical isomers or racemic mixtures, pharmaceutically acceptable salts and prodrug forms. Within its use in context, the term compound may refer to a single compound or a mixture of compounds as otherwise described herein.

The term “modulator” as used herein refers to a compound that serves as an agonist, antagonist or regulator of a GTPase as described herein.

The term “agonist”, as used herein, is meant to refer to a compound or agent that mimics or upregulates (e.g., potentiates or supplements) the activity of GTPase.

The term “antagonist” as used herein is meant to refer to a compound that downregulates (e.g., suppresses or inhibits) at least one activity of a compound, e.g., a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “bioactive agent” refers to any biologically active compound or drug which may be formulated for use in the present invention. Exemplary bioactive agents include the compounds according to the present invention which are used to modulate GTPases and to treat cancer as well as other disease states and/or conditions which are otherwise described herein.

The terms “treat”, “treating”, and “treatment”, are used synonymously to refer to any action providing a benefit to a patient at risk for or afflicted with a disease, including improvement in the condition through lessening or suppression of at least one symptom, delay in progression of the disease or delay in the onset of the disease, etc. Treatment, as used herein, encompasses prophylactic and therapeutic treatment, depending on the context of the treatment used. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal or decrease the severity of disease that subsequently occurs. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the symptoms of a disease state and/or condition; in another embodiment, administration of the compounds according to the present invention is effective to decrease the severity of the disease or lengthen the lifespan of the mammal so afflicted, in the case of cancer, as well as other diseases and conditions that are Ras GTPase driven, including for example, histiocyte disorders (e.g. Rosai-Dorfman disease/sinus histiocytosis with massive lymphadenopathy), Noonan syndrome (NS), Noonan syndrome with multiple lentigines, Leopard syndrome, cardiofacio-cutaneous syndrome, neurofibromatosis type I (NF1) and secondary effects of same including fibromas, scoliosis, long bone dysplasmia, osteoporosis and cognitive impairment, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 and hypertrophic cardiomyopathy (HCM), among others.

The term “pharmaceutically acceptable” as used herein means that the compound or composition is suitable for administration to a subject to achieve the treatments described herein, without unduly deleterious side effects in light of the severity of the disease and necessity of the treatment.

The term “inhibit” as used herein refers to the partial or complete elimination of a potential effect such as a symptom or a secondary condition of a disease state, while inhibitors are compounds that have the ability to inhibit.

The term “prevention” when used in context shall mean “reducing the likelihood” or preventing a condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients of subjects, administration with compounds according to the present invention will reduce the likelihood or inhibit a particular condition or disease state (in particular, the worsening of a disease state such as the metastasis of cancer or other accepted indicators of disease progression in the case of inflammatory and neurologic diseases) from occurring.

The term “cancer” shall refer to a proliferation of tumor cells having the unique trait of loss of normal controls, resulting in unregulated growth, lack of differentiation, local tissue invasion, and/or metastasis. Examples of cancers from which the compounds of the present invention may be used to treat include, without limitation, carcinomas (e.g., squamous-cell carcinomas, basal cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, ovary, pancreas, prostate, and stomach; hematologic cancers, including leukemias, such as acute myelogenous leukemia, acute lymphocytic leukemia, acute promyelocytic leukemia (APL), acute lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; blastomas, including glioblastoma and medulloblastoma (brain tumors), hepatoblastoma (liver tumor), nephroblastoma (kidney tumor), neuroblastoma (neural tumor), osteoblastoma (bone tumor) and retinoblastoma (retinal tumor in the eye), tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line (germ cell) tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas, among others. It is noted that certain cancers such as leukemias, colorectal cancer, pancreatic cancer, lung cancer, lung adenocarcinoma, mucinous adenoma, ductal carcinoma of the pancreas, colorectal cancer, thyroid cancer, salivary duct carcinoma, epithelial-myoepithelial carcinoma, kidney cancer, astrocytoma and melanoma, have been shown are believed to be associated with RAS GTPase modulation (often KRas, HRas or NRas are upregulated or hyperexpressed in the cancer tissue and are principal target cancers for compounds and therapies according to the present invention. The term cancer includes naïve cancers, recurrent cancers, drug resistant cancers and metastatic cancers, including cancer stem cells. In embodiments, the compounds according to the present invention are effective to treat recurrent cancers and/or metastatic cancers and to inhibit and/or reduce the likelihood that a cancer stem cell will grow and elaborate into a more advanced form of cancer.

In addition to the treatment of principally ectopic cancers as described above, the present invention also may be used preferably to treat eutopic cancers such as choriocarcinoma, testicular choriocarcinoma, non-seminomatous germ cell testicular cancer, placental cancer (trophoblastic tumor) and embryonal cancer, among others.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia subsumes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors, among numerous others as described herein) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms (many of which or more are identified above as ‘cancer”) include neoplasms or neoplasias from which the target cell of the present invention may be derived including without limitation, carcinomas (e.g., squamous-cell carcinomas, basal cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly. Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas, among others. See, Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

The term “additional anti-cancer agent” is used to describe an additional compound which may be coadministered with one or more compounds of the present invention in the treatment of cancer. Such agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901 AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736 AVE-0005, the acetate salt of [D-Ser(But) 6, Azgly 10](pyro-Glu-His-Trp-Ser-Tyr-D-Ser(But)-Leu-Arg-Pro-Azgly-NH₂ acetate [C₅₉H₈₄N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951 aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK 186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilumumab, vemurafenib among others, including immunotherapy agents such as IDO inhibitors (an inhibitor of indoleamine 2,3-dioxygenase (IDO) pathway) such as Indoximod (NLG-8187), Navoximod (GDC-0919) and NLG802, PDL1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab and tremelimumab, among others.

The term “GTPase” is used to describe the RAS GTPases, which is a family of GTPases related to RAS family. These include the KRas GTPases, NRas GTPases and the HRas GTPases, including wild-type (WT) and related prevalent mutant forms of these GTPases such as G12A, G12D, G12V, G12C, G13D, Q61R, Q61L, and Q61H Ras (KRas, NRas and Hras) mutant forms. Together, these GTPase proteins are intimate to processes which are related to cancer and its elaboration and are targets for cancer treatment through modulation, in more particular aspects, inhibition of these GTPase targets, GTPase mediates a number of disease states, including cancer, as otherwise disclosed herein, as well as a number of sporadic and genetic diseases including, histiocyte disorders (e.g. Rosai-Dorfman disease/sinus histiocytosis with massive lymphadenopathy), Noonan syndrome (NS), Noonan syndrome with multiple lentigines, Leopard syndrome, cardiofacio-cutaneous syndrome, neurofibromatosis type I (NF1) and secondary effects of same including fibromas, scoliosis, long bone dysplasmia, osteoporosis and cognitive impairment, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 and hypertrophic cardiomyopathy (HCM), among others, including infections such as Entamoeba histolytica, among others.

The term “non-RAS GTPases”, includes the Rho family of GTPases, which is a family of small signaling GTPases, of which Rac1, Cdc42 and RhoA are the most well studied members. These GTPases have been shown to regulate many aspects of intracellular dynamics, and play a role in cell proliferation, apoptosis, gene expression, and other common cellular functions. They consequently have utility in the treatment of sporadic and genetic diseases, as well as cancers in certain embodiments according to the present invention.

The term “co-administration” or “adjunct therapy” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, including sequential administration. Preferably, effective concentrations of all co-administered compounds or compositions are found in the subject at a given time. The term co-administration or adjunct therapy also contemplates other bioactive agents being coadministered with pharmaceutical compositions according to the present invention, especially where a cancer has metastasized or is at risk for metastasis.

The term “sequencing” refers to the process of determining the sequence of a polynucleotide or protein compound which binds to a target protein in the assays according to the present invention. Such polynucleotide or protein to any polynucleotide or protein, including, e.g., a cDNA, cDNA fragment, a genomic DNA, a genomic DNA fragment, and a synthetic DNA, among numerous others. Moreover, certain differences in nucleotide sequences may exist between individual organisms, of the same or different species, which are called alleles. Such allelic differences may or may not result in differences in amino acid sequence of the encoded polypeptide yet still encode a polypeptide with the same biological activity.

The term “fluorescently labeled” is used to describe a protein (e.g. a fused protein), a binding partner of a protein (e.g., a ligand of a protein such as GTP for GTPase) or a compound fluorophore label that is selected such that its emitted fluorescent energy can be detected by fluorimetry, especially including by flow cytometry (e.g. high throughput flow cytometry). The fluorophore label may be a fluorescent protein or dye, e.g., a fluorescent protein as described in Matz et al., Nature Biotechnology (October 1999) 17:969-973, a green fluorescent protein from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304, the disclosures of which are herein incorporated by reference. Fluorescent dyes which may be used to fluorescently label the protein, binding partner or compound other fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, Cy3 and Cy5, macrocyclic chelates of lanthanide ions, e.g. quantum dye, etc., chemilumescent dyes, e.g., luciferases, including those described in U.S. Pat. Nos. 5,843,746; 5,700,673; 5,674,713; 5,618,722; 5,418,155; 5,330,906; 5,229,285; 5,221,623; 5,182,202; the disclosures of which are herein incorporated by reference.

The term “standard” is used to describe binding measurements of known agonists/antagonists/regulators or other ligands with a target protein (e.g. a fused protein or receptor) in an assay such that the binding measurements of the known agonist/antagonist/regulator in the assay may be compared with binding measurements of a compound of unknown activity in the same assay. By comparing the binding measurements of the compound of unknown activity to the target protein with the binding measurements of the known compound to the target protein, a determination may be made as to the activity of the compound of unknown activity as an agonist/antagonist/regulator or a compound which does not bind to the target protein.

Compounds according to the present invention may be readily formulated into pharmaceutical compositions, useful in the treatment of disease states and/or conditions as otherwise described herein. These disease states and/or conditions include immune dysfunction, pigmentation or neurological disorders which occur as a consequence of impaired GTPase function and/or functional insufficiency. Additional disease states and/or conditions which may be favorably influenced by treatment with the present compounds include cancers (e.g., leukemias, colorectal cancer, pancreatic cancer, lung cancer, lung adenocarcinoma, mucinous adenoma, ductal carcinoma of the pancreas, colorectal cancer, among others, often associated with KRAS, thyroid cancer, salivary duct carcinoma, epithelial-myoepithelial carcinoma, kidney cancer, astrocytoma, among others, often associated with HRAS and melanoma, often associated with NRAS), histiocyte disorders (e.g. Rosai-Dorfman disease/sinus histiocytosis with massive lymphadenopathy), Noonan syndrome (NS), Noonan syndrome with multiple lentigines, Leopard syndrome, cardiofacio-cutaneous syndrome, neurofibromatosis type I (NF1) and secondary effects of same including fibromas, scoliosis, long bone dysplasmia, osteoporosis and cognitive impairment, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 and hypertropic cardiomyopathy (HCM), among others, including infections caused by Entamoeba histolytica, among others.

Pharmaceutical compositions comprise an effective amount of one or more compounds according to the present invention in combination with a pharmaceutically acceptable carrier, additive or excipient, optionally in combination with at least one additional anticancer agent.

As noted above, the compounds and method of the invention modulate GTPase as otherwise described herein, and are useful for the inhibition (including prophylaxis) and/or treatment of cancer, sporadic or genetic diseases or conditions and infections, including those caused by Entamoeba histolytica.

In methods according to the present invention, subjects or patients in need are treated with the present compounds, pharmaceutical compositions in order to inhibit, reduce the likelihood or treat a disease state, condition and/or infection as otherwise described herein. The disease states, conditions and infections treated by the present compounds and compositions are readily recognized and diagnosed by those of ordinary skill in the art and treated by administering to the patient an effective amount of one or more compounds according to the present invention.

Generally, dosages and routes of administration of the compound are determined according to the size and condition of the subject, according to standard pharmaceutical practices. Dose levels of compounds employed can vary widely, and can readily be determined by those of skill in the art. Typically, amounts in the milligram up to gram quantities are employed, although in certain instances, amounts above or below that range may also be used. The composition may be administered to a subject by various routes, e.g. orally, transdermally, perineurally or parenterally, that is, by intravenous, subcutaneous, intraperitoneal, intrathecally or by intramuscular injection, among others, including buccal, rectal, and transdermal administration. Subjects contemplated for treatment according to the method of the invention include humans, companion animals, laboratory animals, and the like.

Formulations containing the compounds according to the present invention may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20% by weight or more), and optional pharmaceutical adjuvants, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for modulating GTPase in a subject according to the present invention in a subject.

### EXAMPLES

The present inventor's view is that the recent discovery of a therapeutic approach targeting one RAS mutation (G12C) establishes the premise that screening specific Ras mutant proteins will reveal mutation- and cancer type-specific vulnerabilities for mutation-selective anti-Ras therapies. Additionally, the recent identification of unique pockets and protein-protein interaction interfaces dictate unique behaviors of individual Ras proteins (HRas, KRas and NRas) further supporting the premise that Ras selective compounds will have significant utility. The present invention relates further to our unique multiplexed experimental approach that ensures the stability of Ras and Ras-related GTPase and allows comparative assessment of target sensitivity during screening with compound libraries (methods). The approach has demonstrated utility for detection of hits and development of robust leads that are active against select or multiple GTPases. Through combined testing of off-patent drugs, cheminformatics to identify the most promising scaffolds, and preclinical and clinical testing, two enantio-selective scaffolds derived from off-patent drug libraries were shown to have clinical translational utility. Our publication and patent track records suggest that GTPases are directly druggable targets.

Through a pilot inter-institutional CTSC collaboration, we tested repurposed drugs in a multiplex screen against wild-type KRas and prevalent point mutant proteins (G12A, G12D, G12V, G12C, G13D, Q61R, Q61L, and Q61H). The screen identified KRas and HRas protein selective compounds in primary and dose response secondary screens.

In order to identify and validate mutant KRas proteins as targets for drug repurposing, the inventors prepared 9 distinct, highly purified oncogenic Ras proteins for display on microspheres (See methods and results).

PanActivators of GTP Binding
Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid, Iopanic acid, Menindione, Iopanic acid.

Pan Inhibitors of GTP Binding
Istradefylline, PR-619, N6022, Diffractaic acid, IPA 3, Fisetin, Folic acid, GSK 3787

Selective Inhibitors of GTP Binding to RAS Proteins, but Not Q61 KRAS Mutants
Guanabenz acetate, Chlorprothixene hydrochloride, NSC 663284.

Mixed Activity Modulators of GTP Binding
Ipsapirone, CG109203X (selective activator non-RAS), Beta Lapachone (RAS activator/non-RAS inhibitor), SF 1670 (RAS activator, non-RAS inhibitor), Darapladib (SB480848), PD 198306, Pimethixene Maleate, Oxyquinoline Hemisulfate.

Materials and Methods:

Reagents: All reagents were from Sigma (St Louis, Mo.) unless otherwise indicated. Plastic-ware was from VWR (Radnor, Pa.) and Greiner Bio-One (Monroe, N.C.). Bead sets for multiplex assays were provided by Duke Scientific (Fremont, Calif.) following protocols developed by the NMMLSC^{54,56}. Guanosine 5′-Triphosphate, BODIPY™ FL 2′-(or-3′)-O-(N-(2-Aminoethyl) Urethane), Trisodium Salt (BODIPY FL GTP) and anhydrous DMSO were from ThermoFisher Scientific (Waltham, Mass.). GST-fusion proteins were either from Cytoskeleton, Inc. (Denver, Colo.) or were purified from E. coli as described below. All solutions were prepared with ultra-pure 18 MΩ water or anhydrous DMSO. Flow cytometric calibration beads were from Bangs Laboratories Inc., (Fishers, Ind.) and Spherotech, Inc., (Lake Forest, Ill.). Off patent commercial libraries were purchased from Prestwick Chemical (Illkirch-Graffenstaden, France), SelleckChem (Houston, Tex.), Spectrum Chemical (New Brunswick, N.J.), and Tocris Bio-Science (Bristol, UK). We also purchased a collection of on patent drugs from MedChem Express (Monmouth Junction, N.J.) that was specifically assembled by UNM collaborators. All purchased libraries were provided as 10 mM stock solutions in 96-well matrix plates except the MedChem Express library which was provided as individual powders that were subsequently solubilized in DMSO. All libraries were reformatted using a Biomek FX^{P} laboratory automated workstation into 384-well plates for storage (Greiner #784201; Labcyte #PP-0200). Low volume dispensing plates (Labcyte #LP-0200) were assembled using an Agilent BioCell work station (Santa Clara, Calif.).

Expression and purification of GST-fusion proteins: Human KRAS4B cDNA sequences encoding the G domains of wild-type and mutant KRAS (G12D, G12A, G12V, G12C, G13D, Q61R, Q61L, and Q61H) fused with glutathione S-transferase were generously provided by the National Cancer Institute through the Ras initiative. The constructs have a Tev protease cleavage site that leaves an extra Gly on the KRas amino terminus after cleavage. The presence of this additional glycine does not alter activity, structure, or other properties measured by NCI. Through an MTA, NCI offers a complete suite of KRAS, HRAS and NRAS clones bearing mutations that can be assessed as needed⁵⁷. Vector options include a T7 promoter based GST-fusion or a T7 promoter based His6-GST-fusion that allows testing of purified proteins with both bead types.

Proteins (residues 1-169) were subcloned into a pET21 vector that adds an N-terminal 6-histidine tag and a TEV protease cleavage site for expression of recombinant protein in Escherichia coli BL21 (DE3) cells (Novagen). The mutations were subsequently verified by DNA sequencing. E. coli BL21 (DE3) cells were grown at 37° C. in Luria-Bertani (LB) medium supplemented with ampicillin and chloramphenicol until A600 of ˜0.5. The temperature was then lowered to 18° C., and GST-KRAS expression was induced with 0.5 mM isopropyl-β-D-1-thiogalactopyranoside (IPTG) after 30 min. The cells were grown for an additional 15 h at 18° C. The cells were then harvested and pelleted at 4000 rpm, resuspended in a lysis buffer (20 mM HEPES, 500 mM NaCl, 1 mM MgCl2, 20 mM imidazole, 5% glycerol (pH 7.75), and protease inhibitor phenylmethanesulfonyl fluoride (ACROS Organics), and sonicated. The cell lysate was centrifuged at 15,000 rpm, and the supernatant was isolated. KRAS proteins were purified using glutathione-agarose affinity chromatography (Qiagen), and were collected by application glutathione to the column. If needed, KRAS proteins were further purified by size exclusion chromatography using a Sephadex G-75 column. Protein purity of >95% was obtained and verified by SDS-PAGE analysis. GST-fusion proteins were stored at −80° C. in 20 mM HEPES, pH 7.4; 50 mM NaCL; 5 mM: MgCl2; 10% glycerol; 10-100 uM GDP. After their initial use, protein preparations were snap-frozen with liquid nitrogen (LN2). Subsequently, single-use aliquots were stored at −30° C. in buffer containing 40% glycerol and 20 uM GDP.

Bead Coupling: 4 μm diameter, glutathione-beads (GSH-beads), distinguished by ten different intensities of red fluorescence (varying by several orders of magnitude of emission at 665±10 nm with excitation at 635 nm) were obtained by special order from Duke Scientific Corp. Each polystyrene bead set is supplied at 1.4×10⁸ beads/ml with approximately 1.2×10⁶ glutathione sites per bead (determined by using GST—green fluorescent protein⁴²). Prior to incubation with individual GST-GTPase fusion proteins, an appropriate volume of each bead slurry was incubated for 30 minutes at room temperature in assay buffer (20 mM HEPES pH 7.5, 125 μM (NH₄)₂SO₂, 1 mM MgCl₂, 0.5 mM EDTA pH 8.0, 0.01% NP-40) supplemented with 1 mM dithiothreitol and 0.1% BSA. Following this initial incubation, passivated beads were collected by centrifugation and resuspended in assay buffer with individual GST-GTPase fusion proteins at a final concentration of 1 M. Overnight coupling reactions were carried out at 4° C. with rotation. The amount of each bead type used for coupling and the final volume of the coupling reaction was determined by the experimental protocol. Typically, bead sets are used at a final concentration of 200 bead/μL. To remove unbound GST-GTPase, bead sets were washed twice by centrifugation with ice-cold assay buffer. Individually coated beads were pooled together and kept cold in an appropriate volume before use in binding assays. Bead-bound proteins are sufficiently stable to study secondary interactions between GTPase-GST chimera's and Bodipy-FL GTP⁵⁴. Note that an extra GSH-bead set is included that is not protein coupled. This set serves as a ‘scavenger’ for proteins that might dissociate during the binding assay.

Dose Dependent Bodipy FL-GTP Binding Assays: For dose dependence binding assays, GTPase-coupled bead sets were incubated on a rotator for 1 hour at 4° C. with varying concentrations of Bodipy FL-GTP (0.3-300 nM). Total assay volume was 10 μL when performed in a microliter plates and 50 μL when performed in tubes. At these concentrations of beads, the concentration of each GTPase in a given reaction is approximately 300 pM. Non-specific binding of Bodipy-FL GTP was assessed by incubating coupled beads with excess GTP prior to the addition of the Bodipy analogue. Dose dependent binding assays were performed in both single- and multiplex format. When assays were performed in plates, the plate assembly was completed as described below.

Assay Plate Assembly: Plate assays were performed in 384-well microtiter plates (Greiner Bio-one, #784101); plates were assembled using a BioTek MultiFlo™ Microplate Dispenser. For dose-dependent Bodipy FL-GTP binding assays, total and non-specific binding were assessed by pre-incubation (30 min/4° C.) in the absence or presence of 30 μM GTP, respectively. This was followed by the addition of varying concentrations of Bodipy FL-GTP. Plates were protected from light and incubated on a rotator for 60 min at 4° C.

For assay screening plates, compound libraries were first dispensed into columns 3-22 using a Labcyte 555 Echo Acoustic Dispenser (San Jose, Calif.) for a final concentration of 10 μM. An equal volume (10 nL) of DMSO was added to the vehicle control wells (column 2). Following the addition of library compounds, 2 μL of assay buffer was added and the plates were mixed before addition of 5 μL of the protein-coupled bead mixtures; 10 μL of assay buffer was added to empty wells in columns 1 and 24. Empty wells serve as wash wells between compound wells and control wells. Plates were mixed and incubated on a rotator for 30 minutes at 4° C. before the addition of 3 μL of Bodipy FL-GTP resulting in a final concentration 10 nM Bodipy FL-GTP. Plates were mixed and incubated for 1 hour at 4° C. Negative controls, containing bead mixtures, Bodipy FL-GTP, and 30 μM unlabeled GTP were assayed separately. Dose response plates were assembled similarly. In this instance, test compounds were added to dose response plates using a dilution protocol of the acoustic dispenser that resulted in a final concentration range of 100-0.015 μM.

Data Acquisition: Assay plates were sampled using the HyperCyt™ high throughput flow cytometry platform (Intellicyt; Albuquerque, N. Mex.). During sampling, the probe moves from well to well and samples 1-2 μL from each well with 0.4 sec transit time in the air before sampling the next well. The resulting sample stream consisting of 384 bubble-separated samples is delivered to an Accuri C6 flow cytometer (BD Biosciences; San Jose, Calif.). Bodipy FL-GTP fluorescence is excited at 488 nm and detected with a 533/30 bandpass filter. Plate data are acquired as time-resolved files that are parsed by software-based well identification algorithms, segregating individual well data that is merged with compound library files to determine compound activity in each well. Gating based on red fluorescence emission distinguishes the separately coated beads. Plate performance was validated using the Z-prime calculation⁵⁸.

Compounds that satisfied the hit selection criteria in the primary screen (change in % binding of 50% from baseline) were cherry-picked from compound storage plates and tested to confirm activity and determine potency. Dose response data points were fitted by Prism® software (GraphPad Software Inc., San Diego, Calif.) using nonlinear least-squares regression in a sigmoidal dose-response model with variable slope, also known as the 4-parameter logistic equation. Curve fit statistics were used to determine the concentration of test compound that resulted in 50% of the maximal effect (EC50), the confidence interval of the EC50 estimate, the Hill slope, and the curve fit correlation coefficient.

Equilibrium Kinetics Assays: GTPase-coupled beads were prepared as described above and were kept on ice until used. Reactions were performed at room temperature in amber micro-centrifuge tubes in an appropriate volume of assay buffer. Bead mixtures were initially incubated on a rotator with Guanabenz acetate (100 μM) or DMSO for 30 min at 4° C. prior to the addition of 1 nM Bodipy FL-GTP. Real-time binding kinetics was recorded using an Accuri C6 flow cytometer. Binding association was followed for 15 minutes at which time excess GTP was added. Disassociation kinetics was followed for an additional 25 minutes. Data was analyzed using GraphPad Prism software. The association time course was fitted to a two phase exponential association and the dissociation time course was fitted to a two phase decay (exponential).

Results

The success of previous studies set the stage for identifying new chemical entities as well as repurposed drugs that inhibit oncogenic KRas proteins to support the notion of Ras GTPases as druggable targets. In order to identify and validate mutant KRas proteins as targets for drug repurposing, we have: 1) prepared 9 distinct, highly purified oncogenic Ras proteins for display on microspheres for multiplex analysis; 2) undertaken semi-quantitative studies for optimizing bead-based display and small molecule screening; 3) identified approved drugs that regulate nucleotide binding to these proteins using multiplex screening and dose-response follow-up technologies; 4) performed proof-of-principle mechanism of action studies as well as initial validation of cellular activities. Multiplexing used UNM's patented GSH bead-GST fusion protein technology⁵⁹. The NCI RAS initiative provided sub-cloned Entry clones into GST E. coli vectors.

Construction and Performance of Multiplex Display GTPases

a) Optimization of Buffer Conditions.

The assay for GTP binding uses a GTP analogue tagged with Bodipy. Previous studies with these conjugates have determined that their fluorescence yield is significantly enhanced when bound within the GTP pocket^{60,61}. Our initial experiments with GST-KRas proteins failed to detect significant binding of Bodipy FL-GTP to KRas WT, G12d, and G13d whereas binding to the G12V mutant was marginally detectable in a magnesium-free buffer used previously in a high throughput campaign to screen non-KRas proteins⁴². We have noted similar changes in detectable binding of Bodipy FL-GTP by other Rho family GTPases when Mg²⁺ was replaced by EDTA⁵⁴. This prompted a review of 5 buffer conditions based on team experience and literature resulting in the use of a buffer that contained both EDTA and Mg²⁺.^{45,52,54-56}. Similar observations regarding Mg²⁺ and EDTA have been reported by Korlach et al.⁶¹

b) Stability; The initial set of proteins (KRas WT, G12v, and G13d) was tested under different storage conditions (−80° C. vs a glycerol/GDP storage at −30° C.) with the 30% glycerol/20 uM GDP storage condition chosen to minimize loss of function during storage and freeze-thaw cycles. Stability of HRas is significantly improved when stored in the presence of GDP³². It should be noted, however, that useful lifetimes for these GST chimeric proteins was still limited to several weeks after the initial thaw of stock material. The inventors were not able to establish satisfactory conditions for storage of the G13D mutant. This may be due to the particular attributes of this ‘fast cycling’ KRas mutant⁶². Once these conditions were chosen, we evaluated dose-dependent binding of Bodipy FL-GTP using established GTP binding procedures.⁵⁵

Over the course of the screening the library plates the KRas proteins remained relatively stable. The KRas proteins were thawed and aliquoted and stored in the −30° C. freezer with glycerol and GDP. After the initial thaw of the proteins, at the beginning of the library screens, there was a 20-30% decrease in the activity of the proteins. For the next 5 weeks the proteins remained consistent at this activity while running the rest of the compound libraries. After this point the KRas proteins decrease down to around 40% when running the Cherry Pick screens.

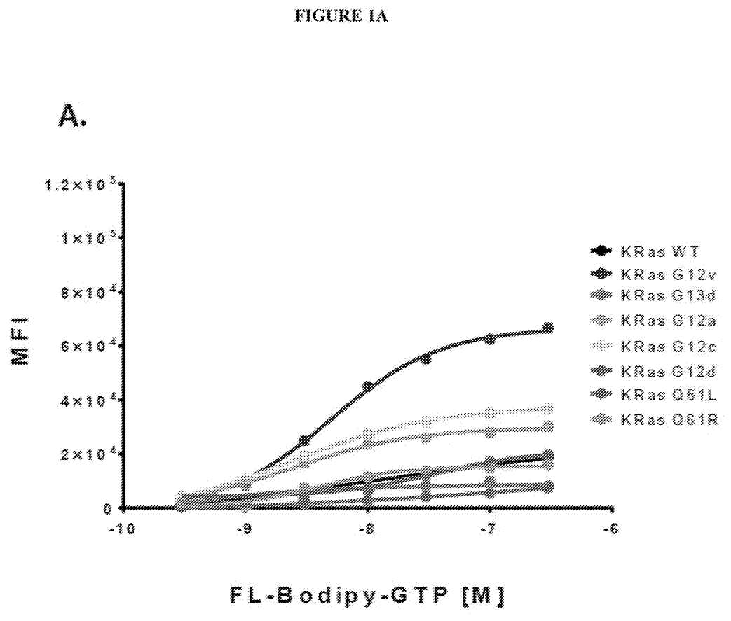

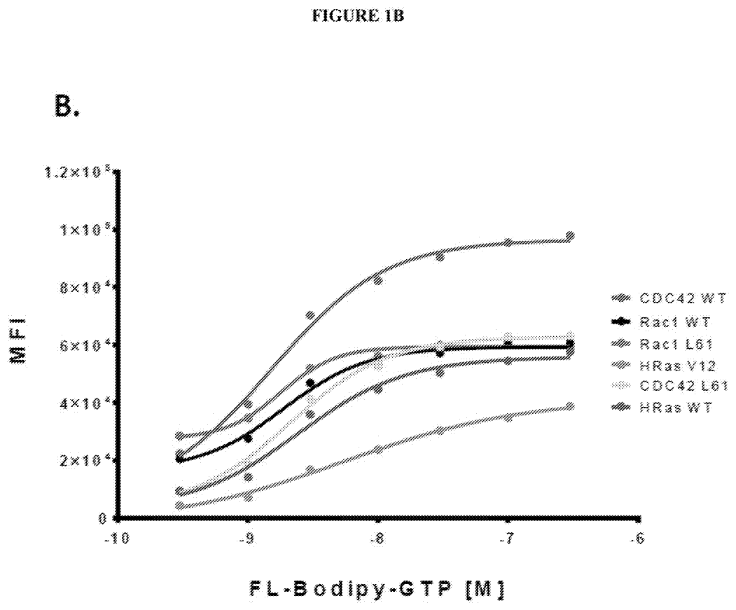

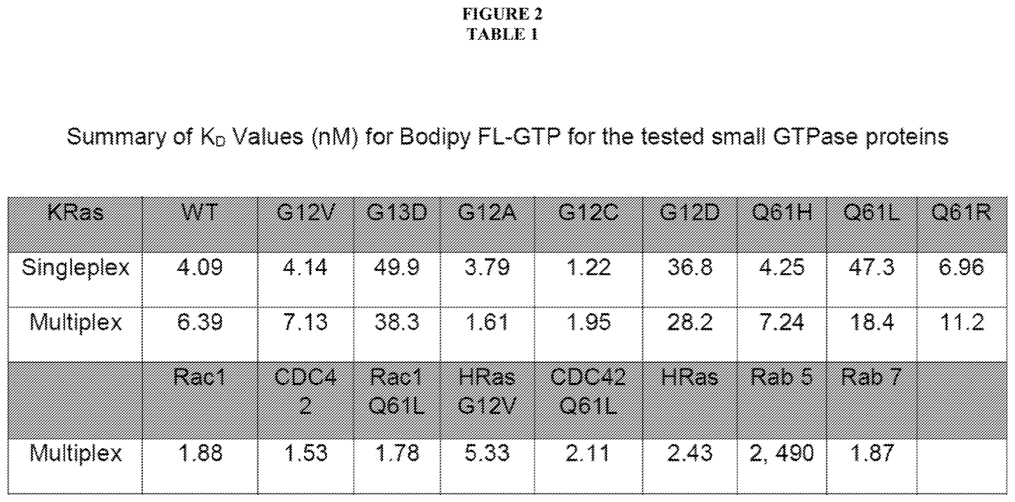

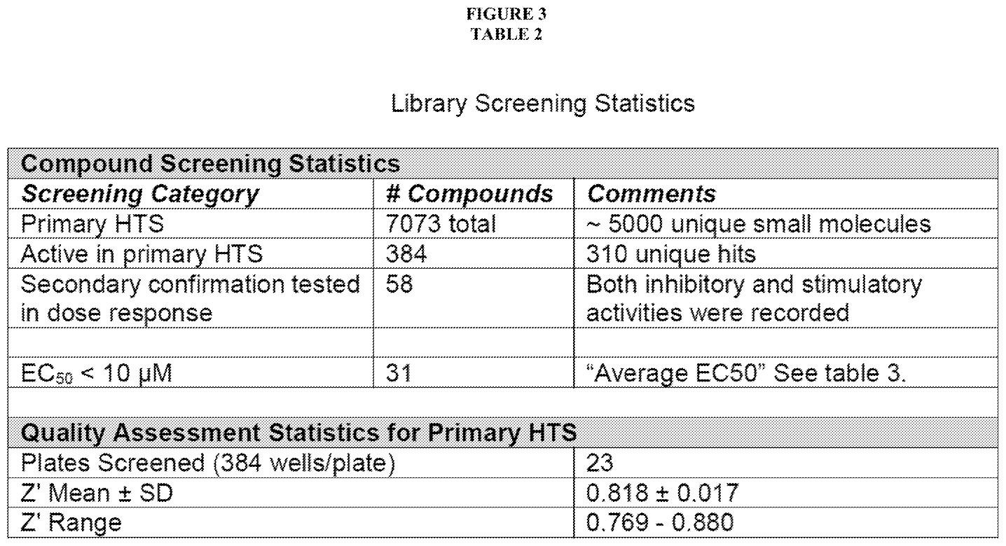

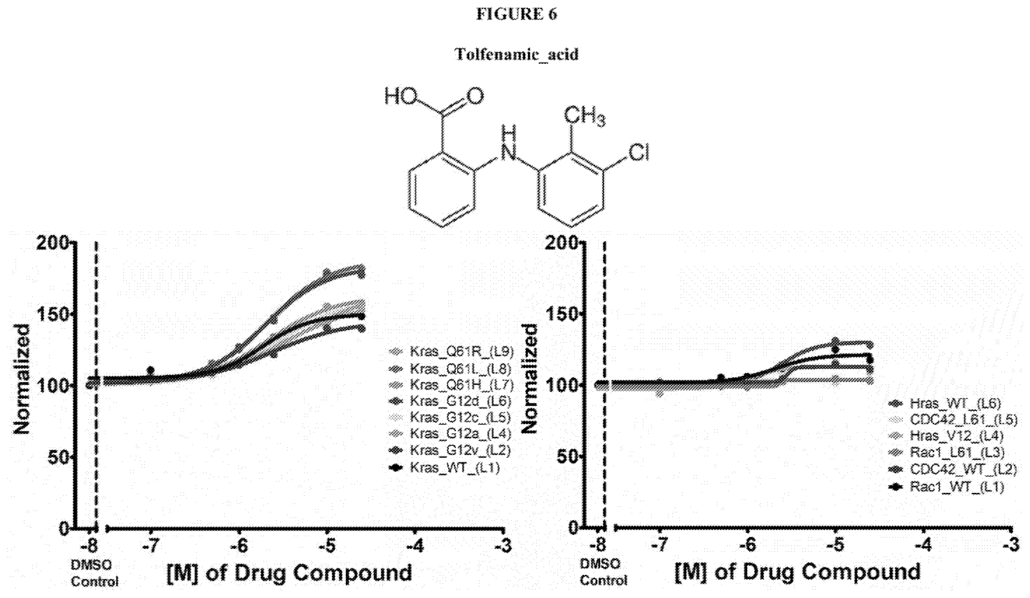

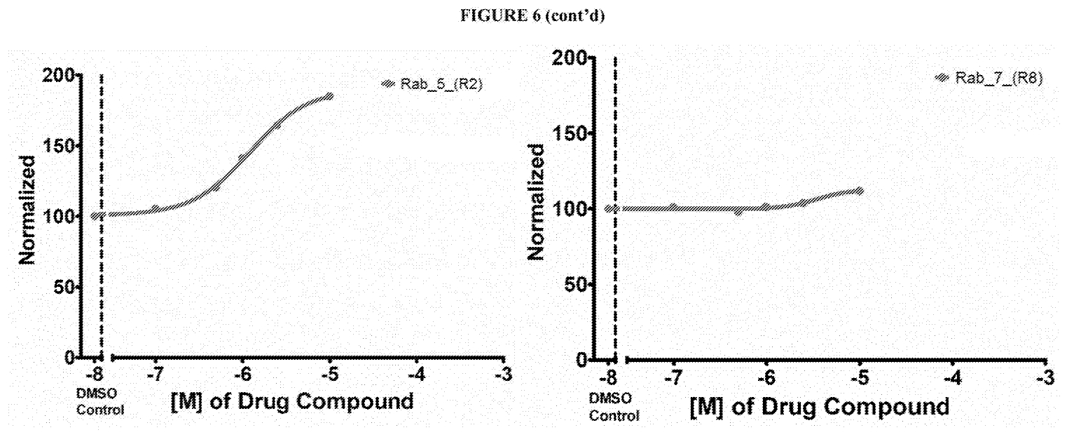

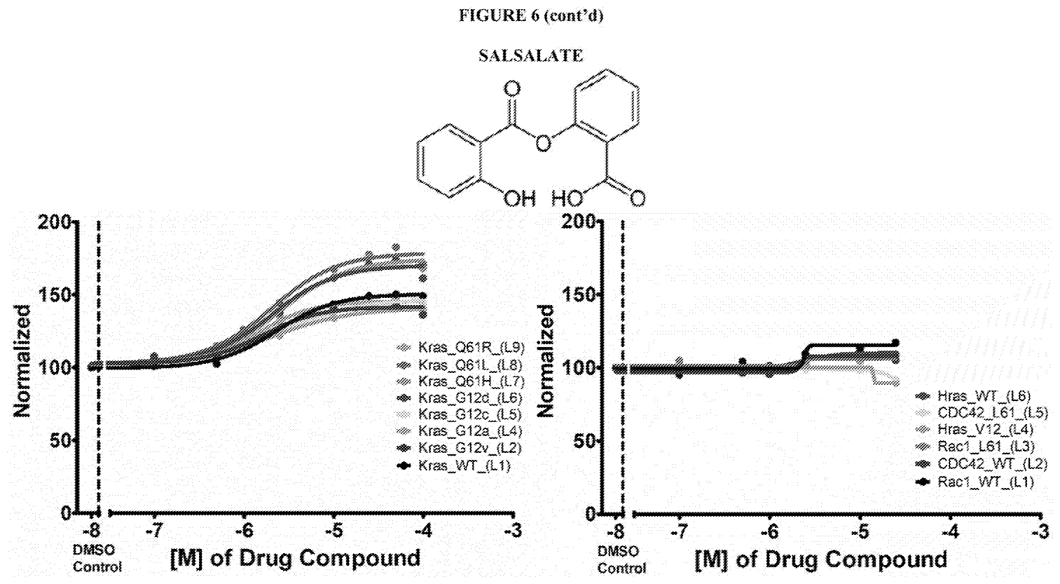

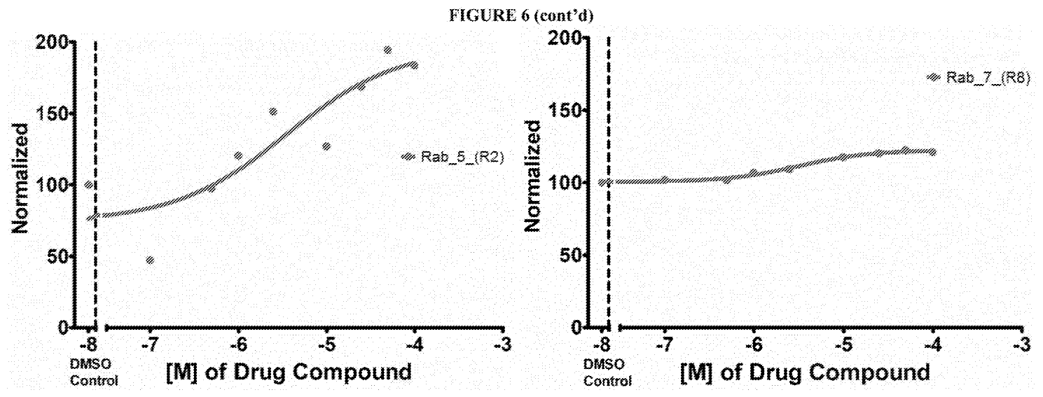

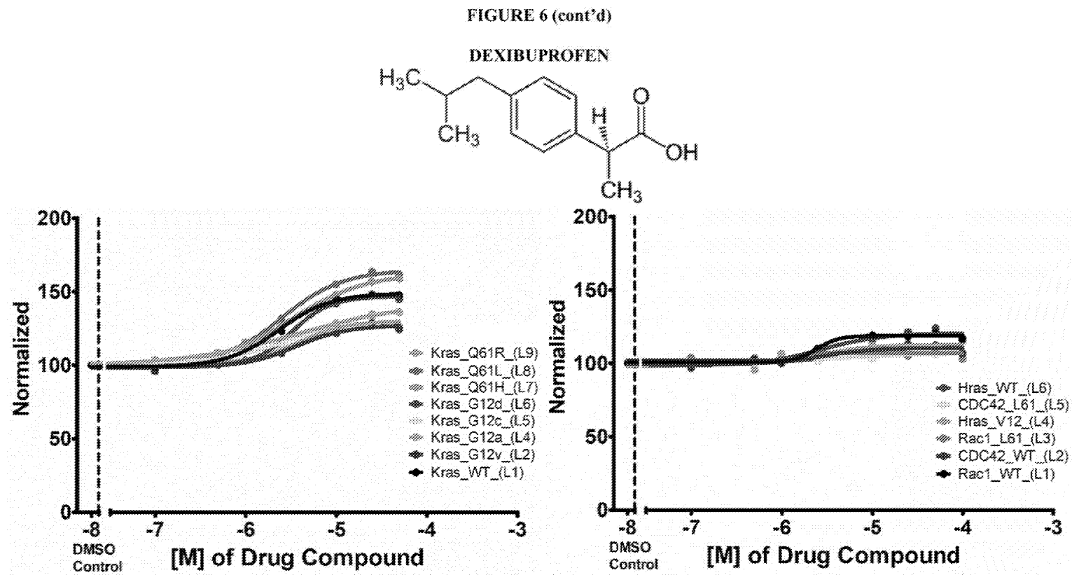

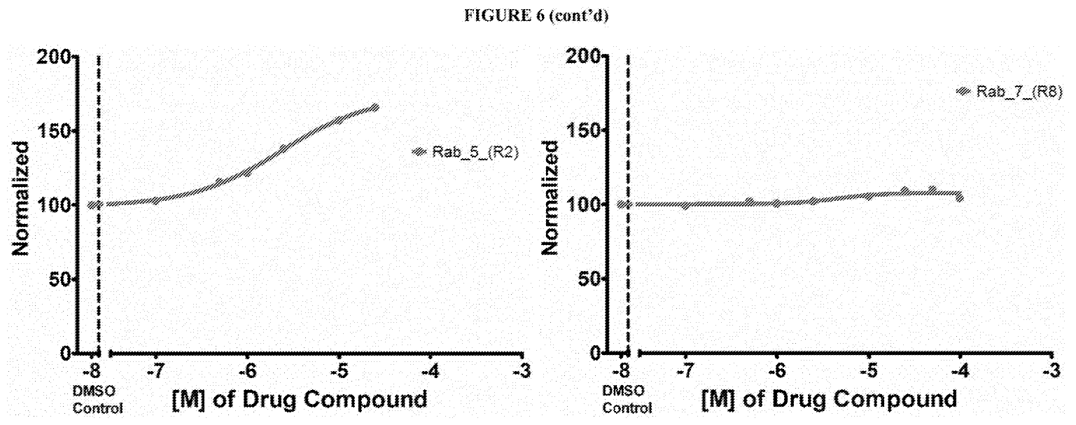

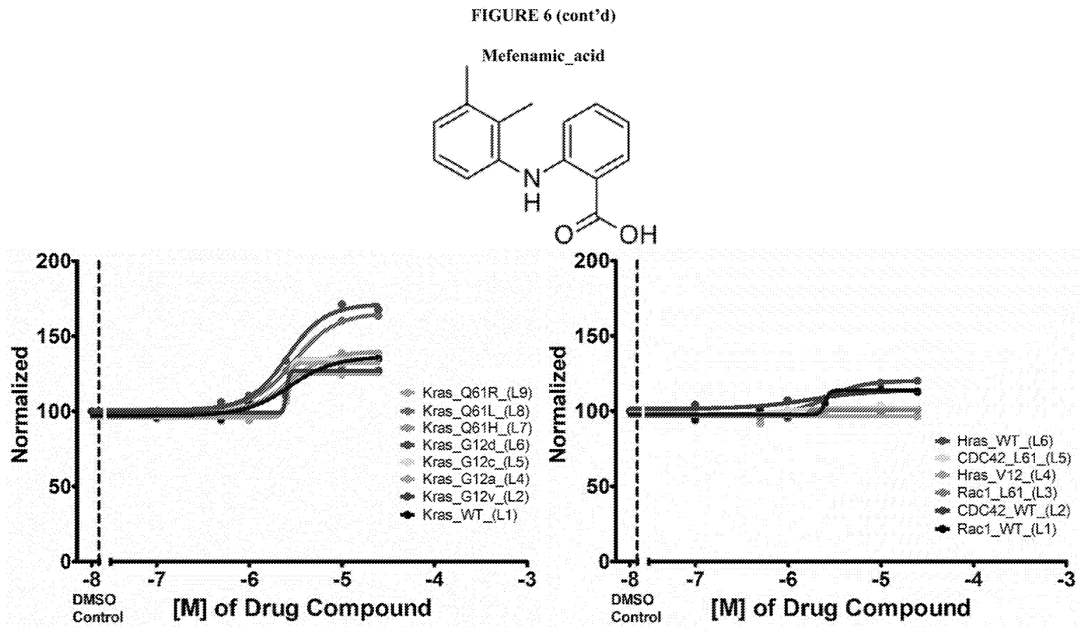

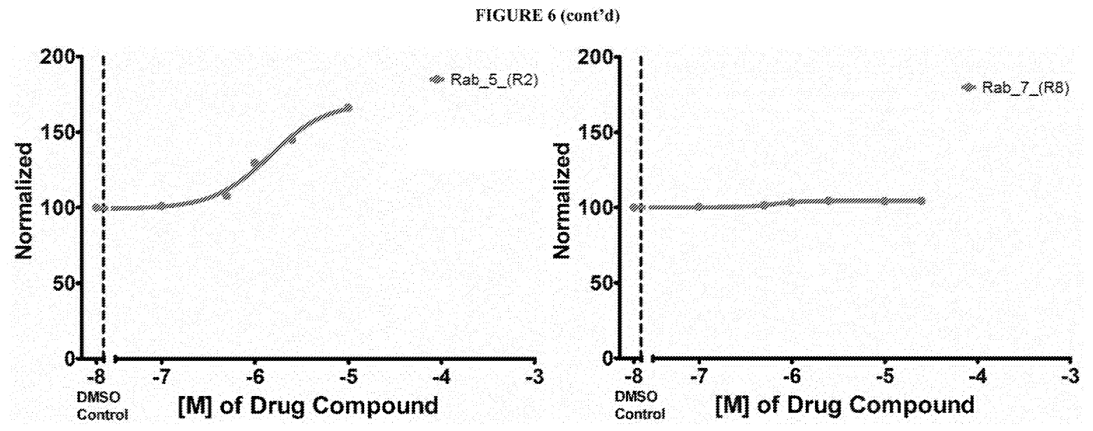

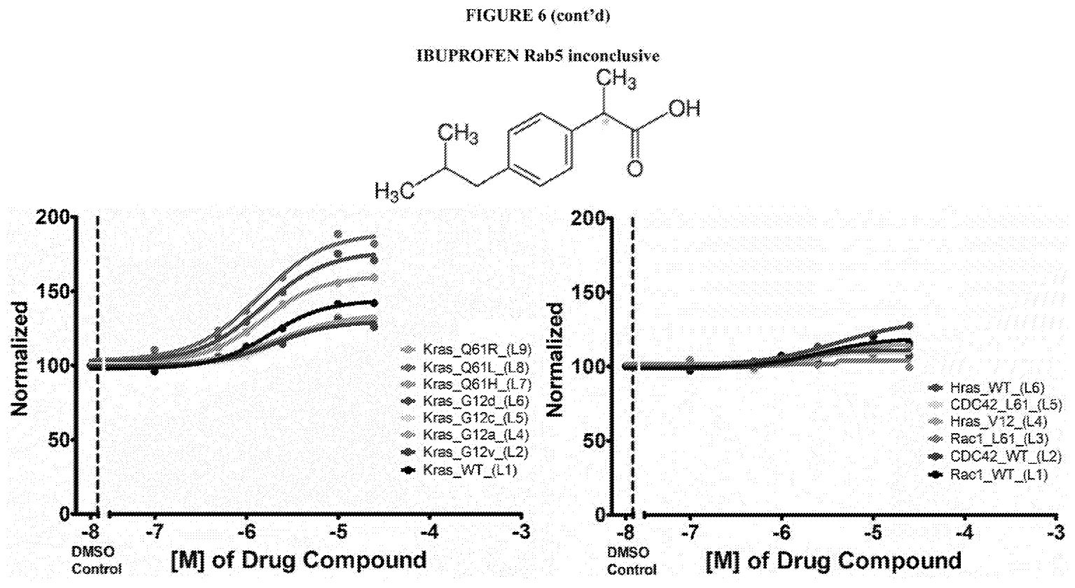

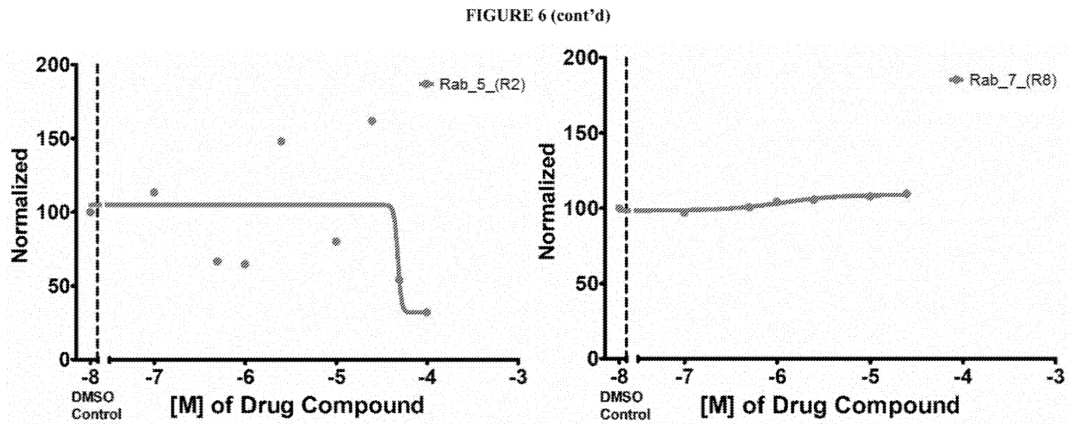

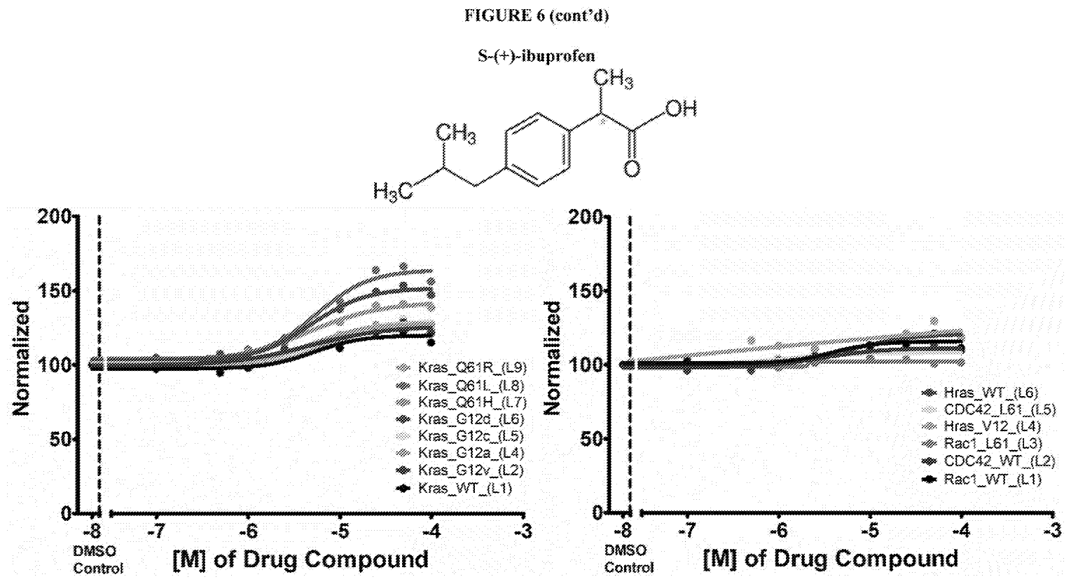

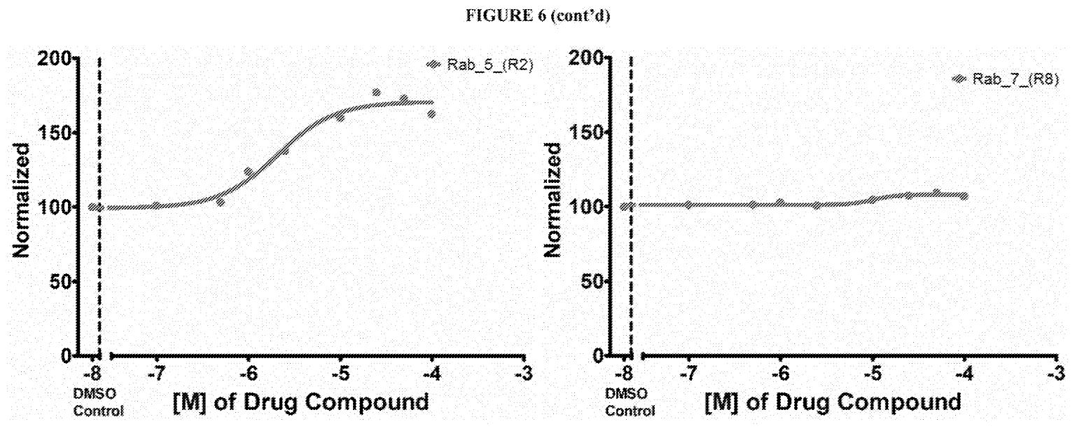

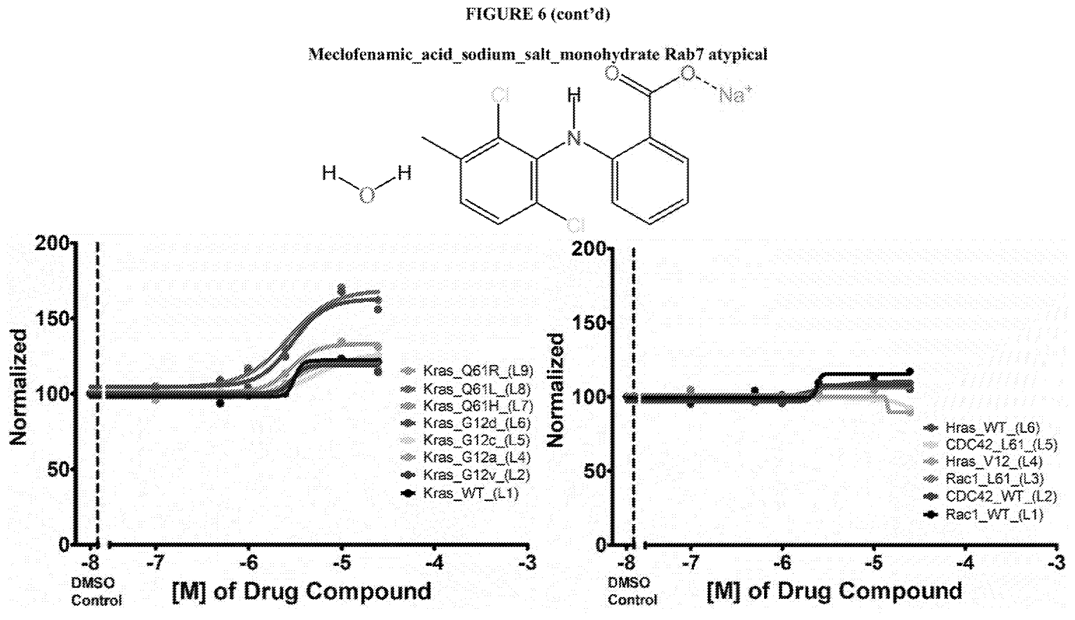

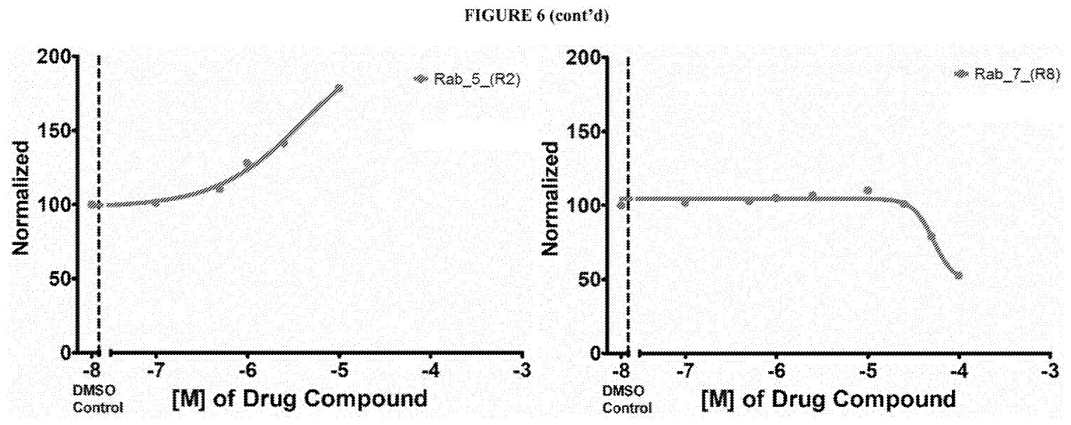

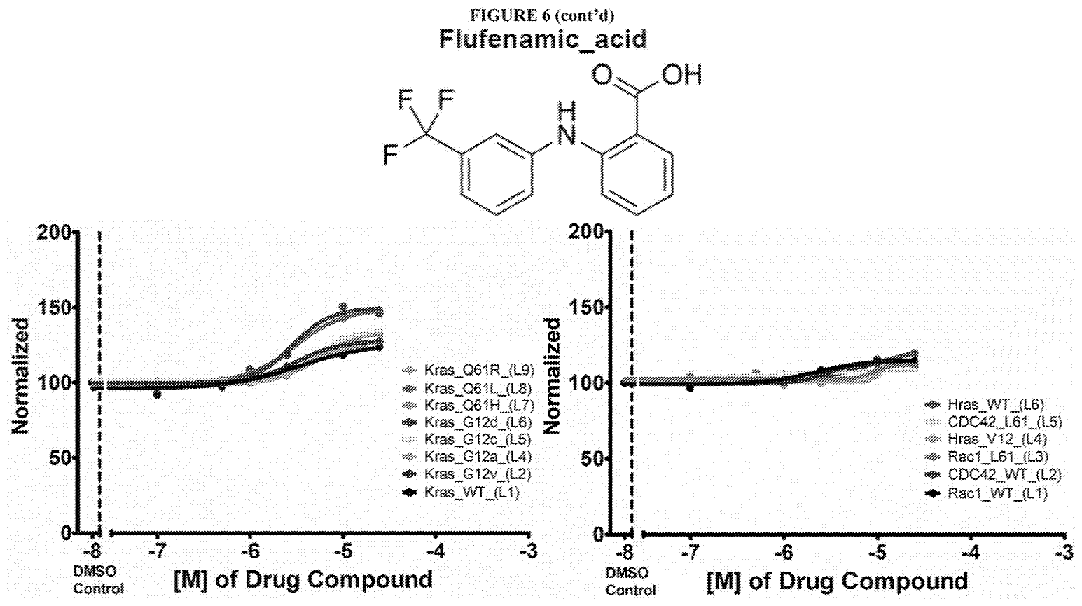

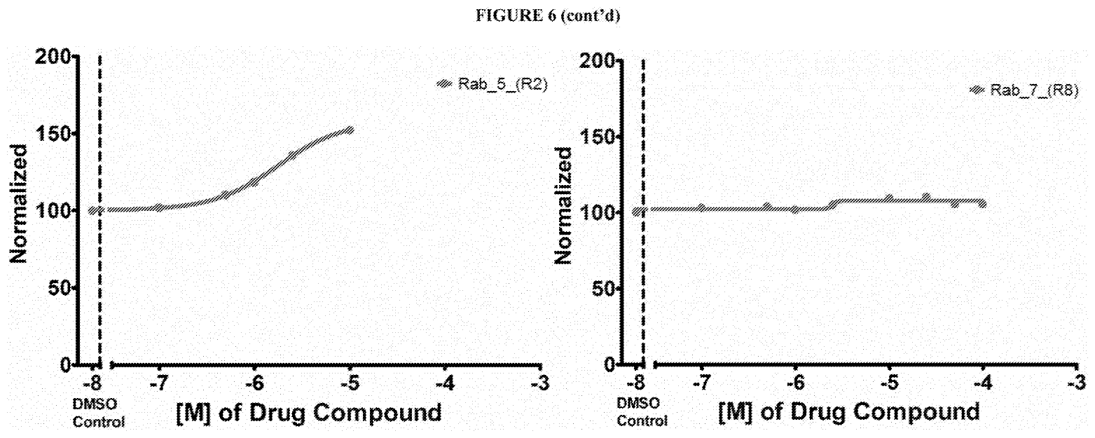

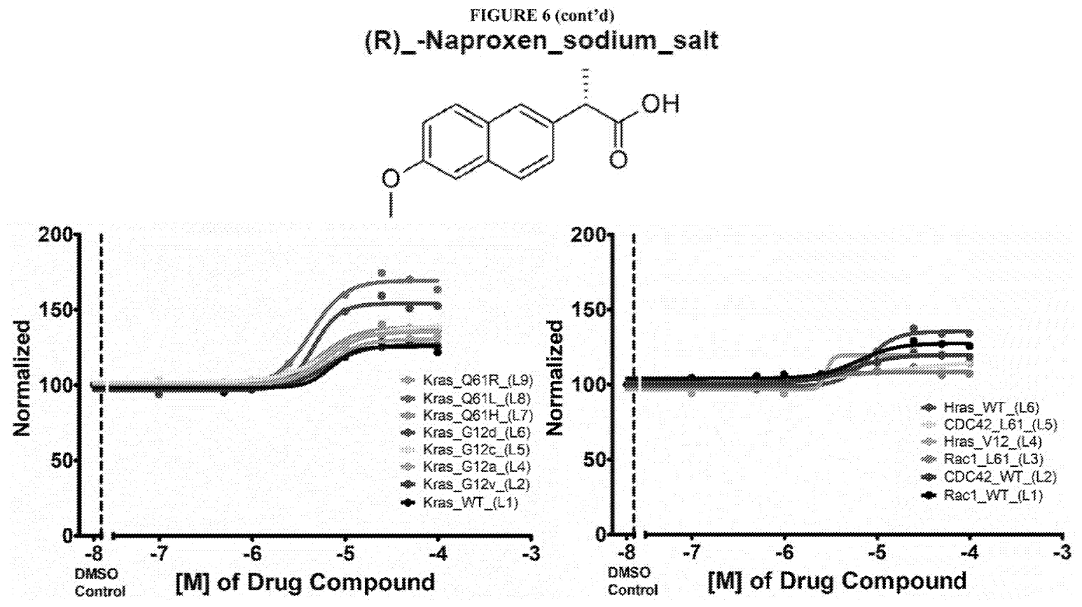

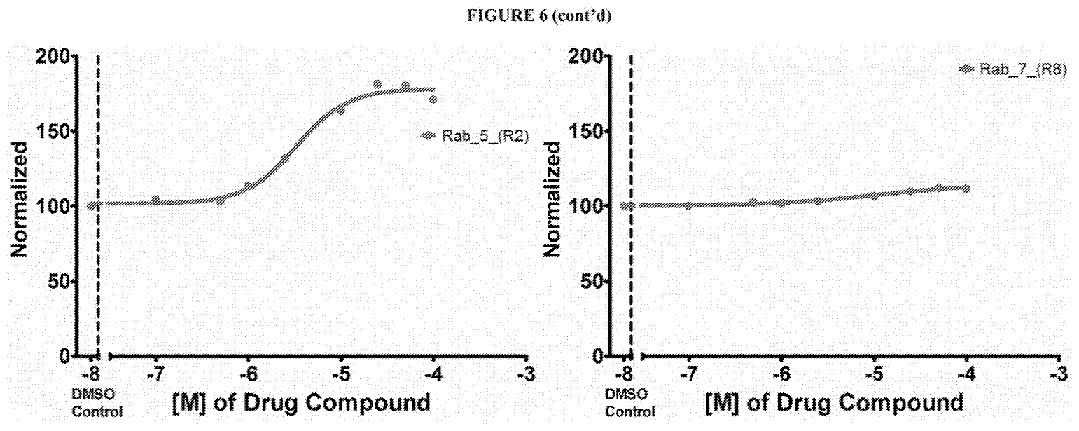

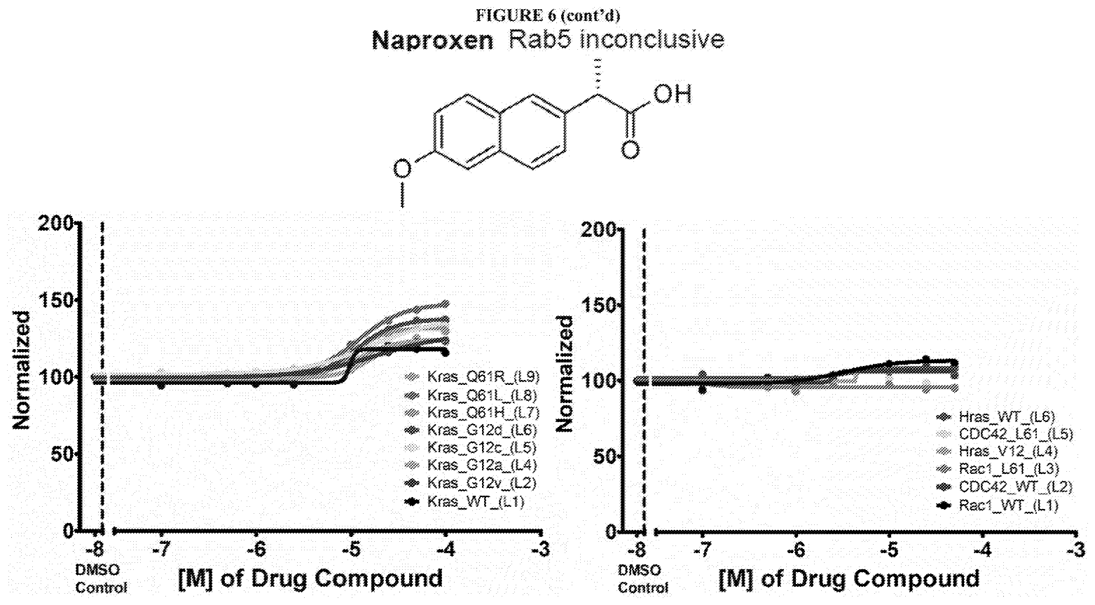

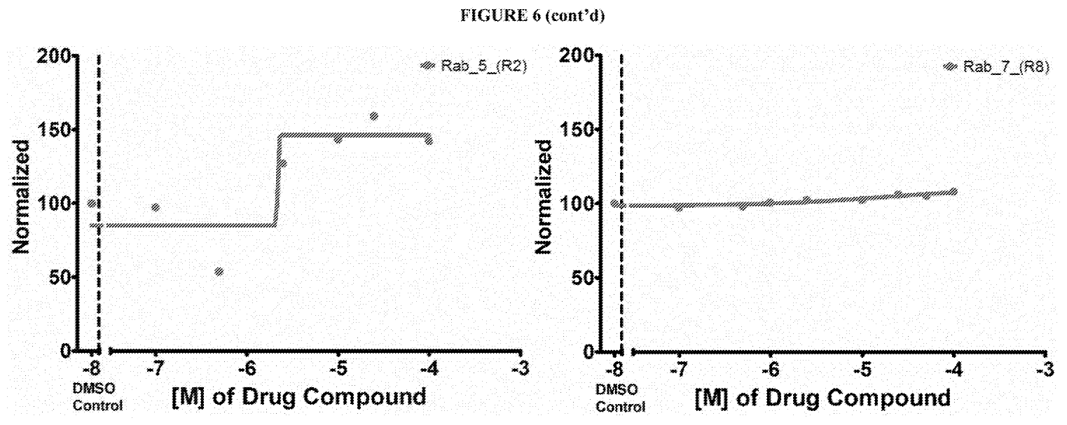

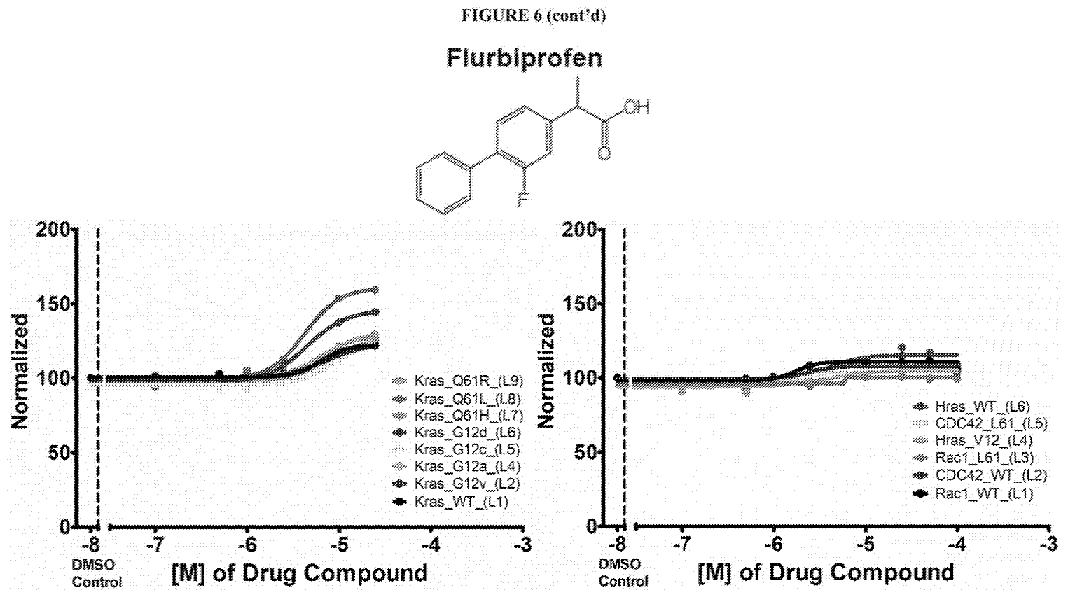

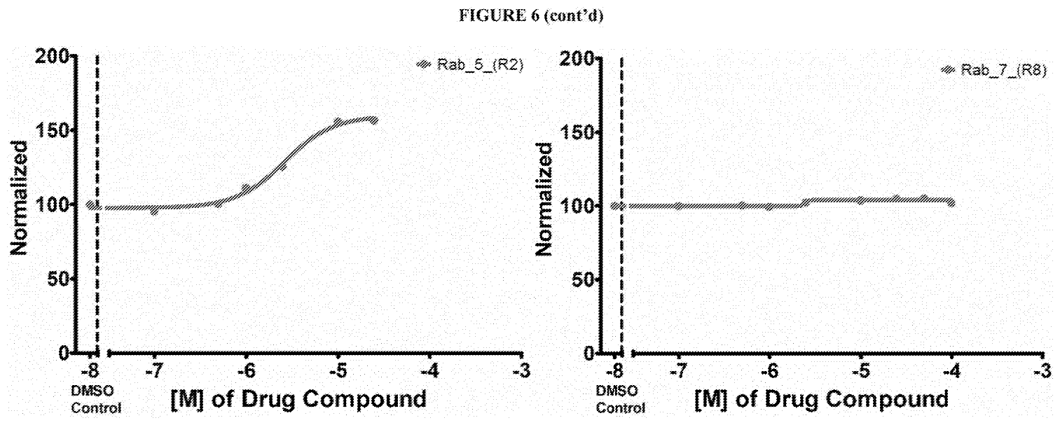

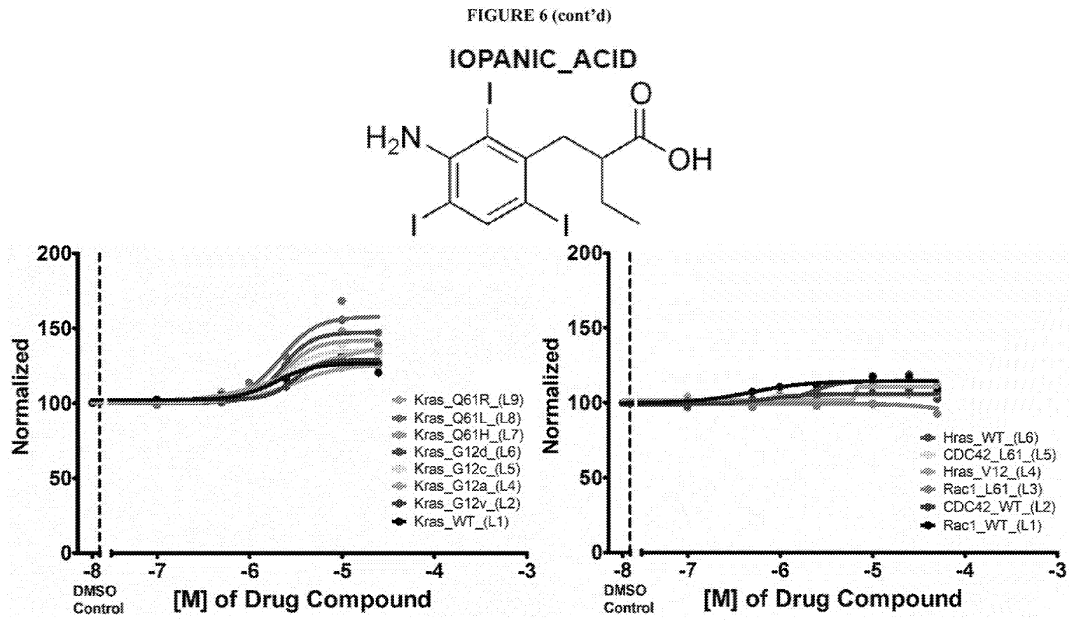

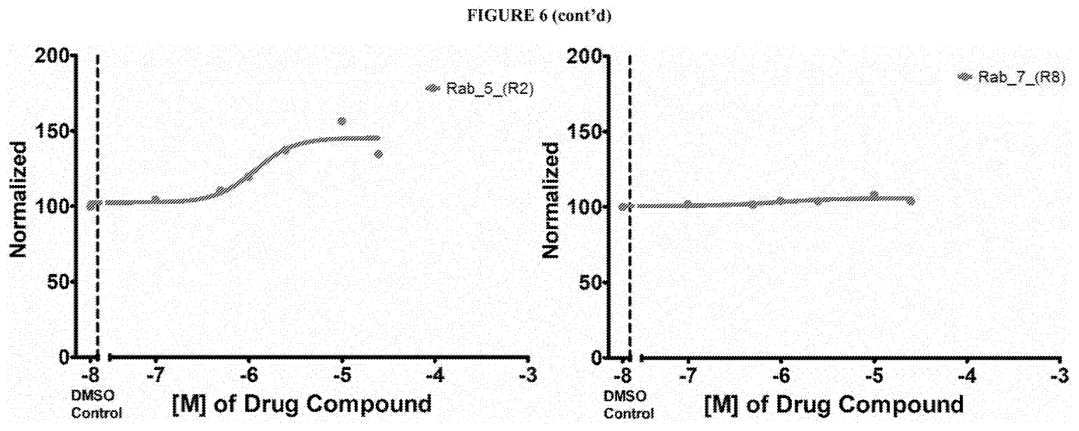

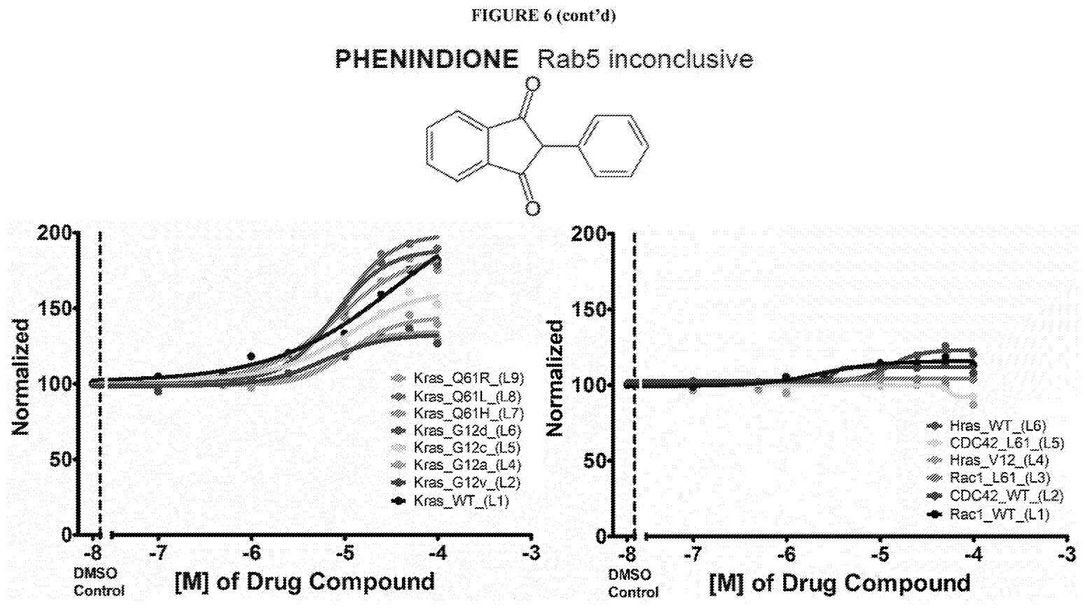

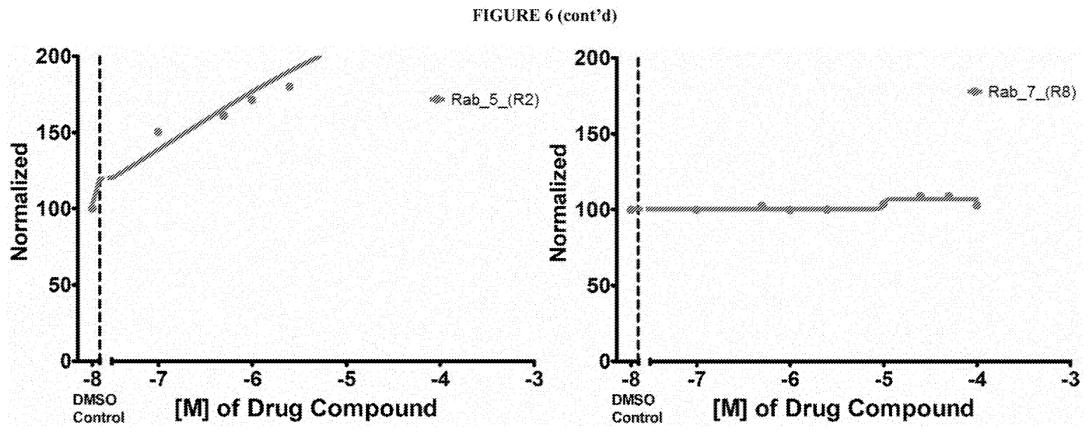

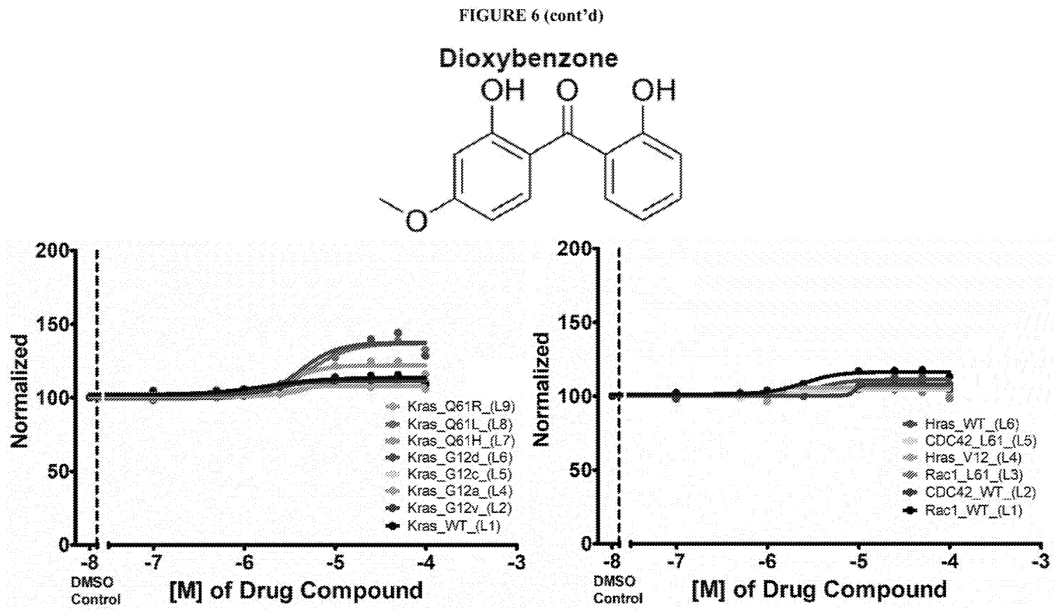

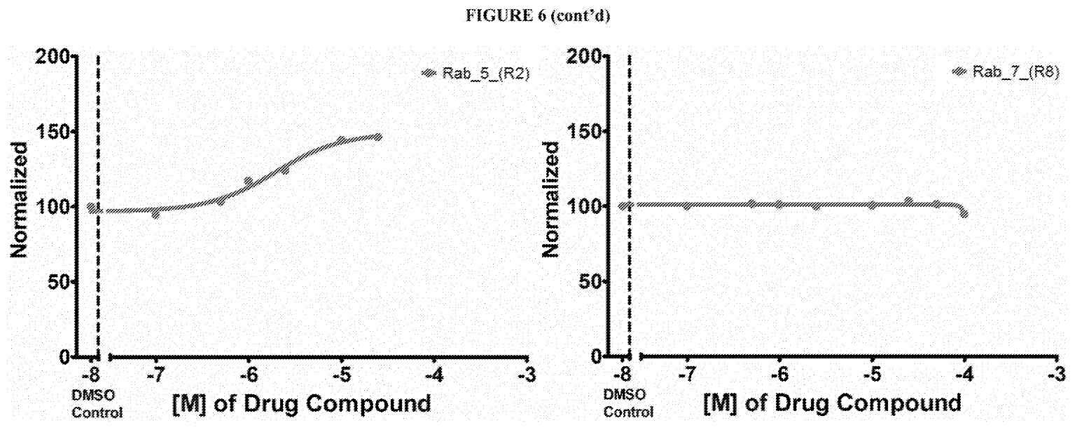

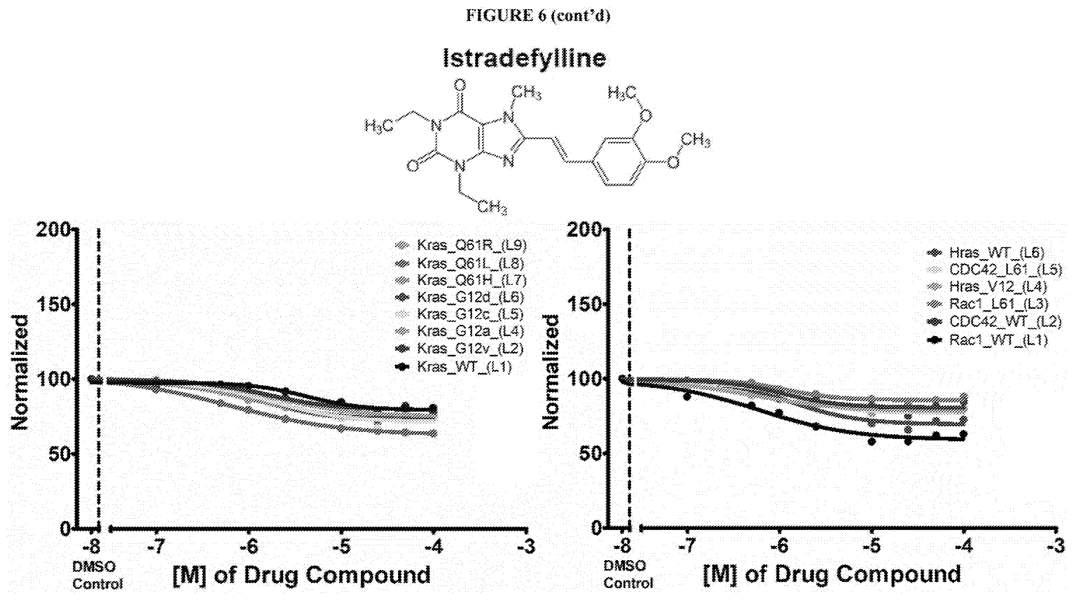

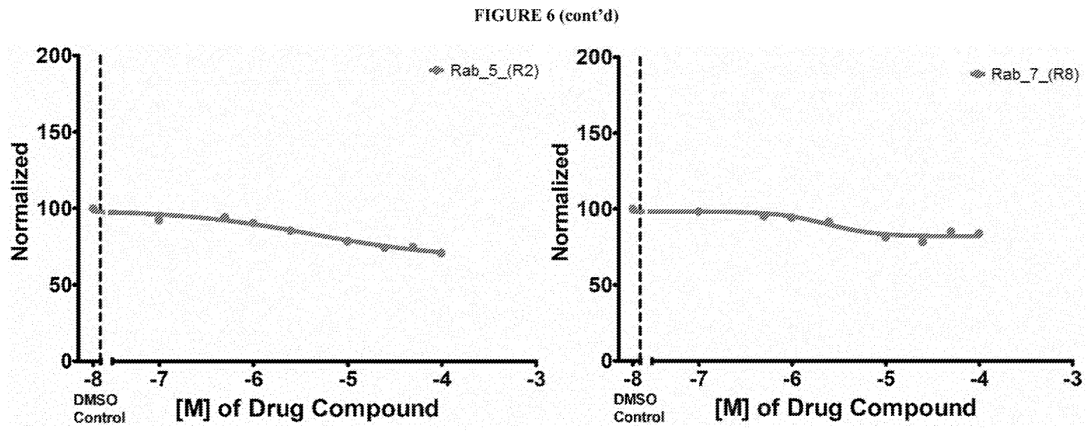

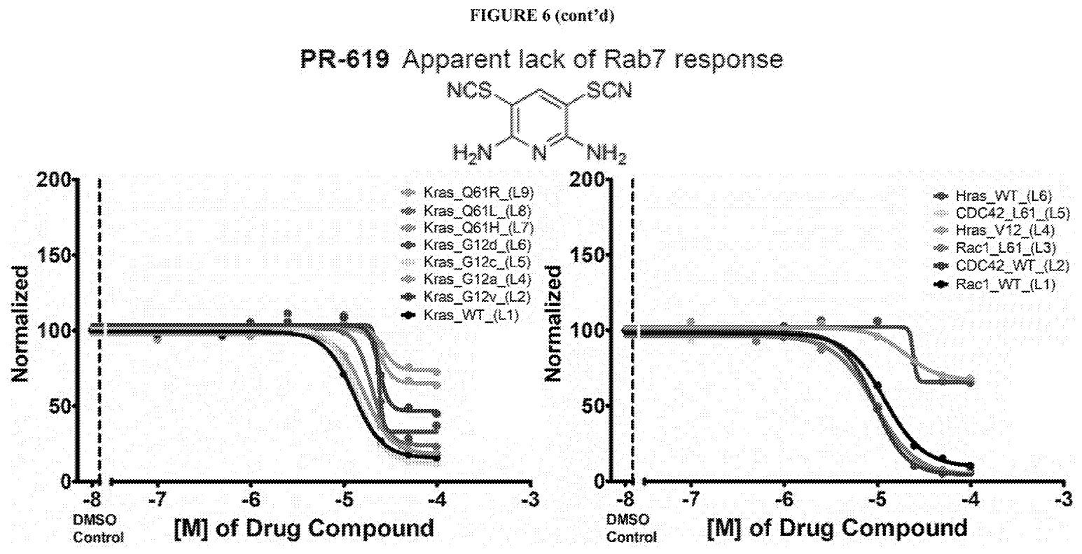

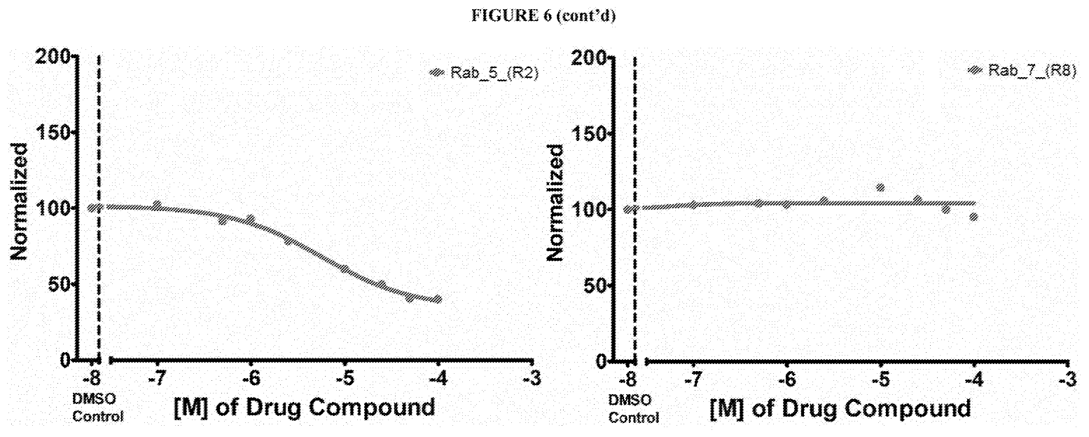

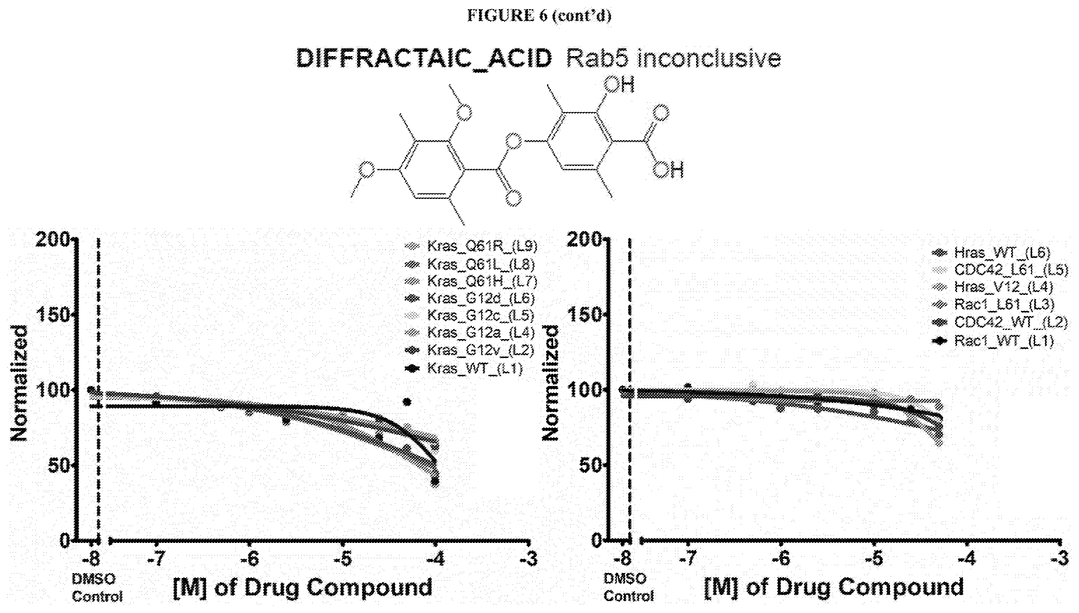

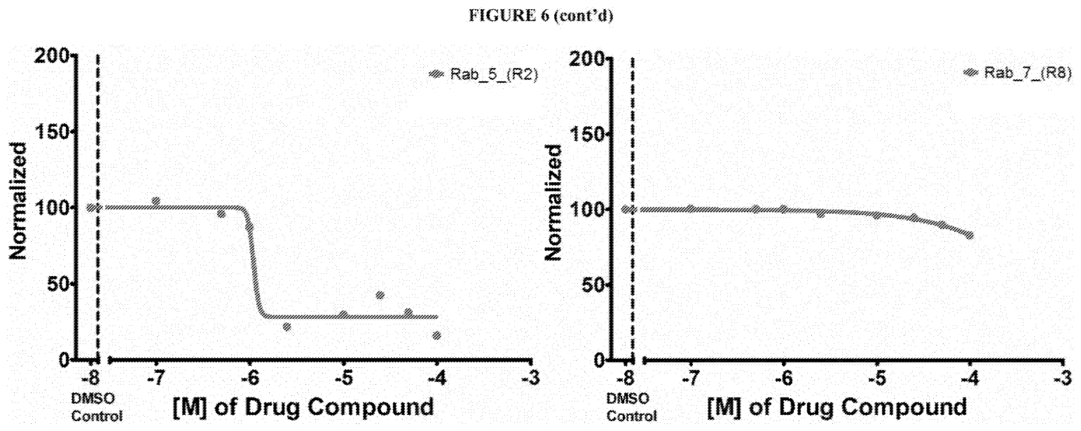

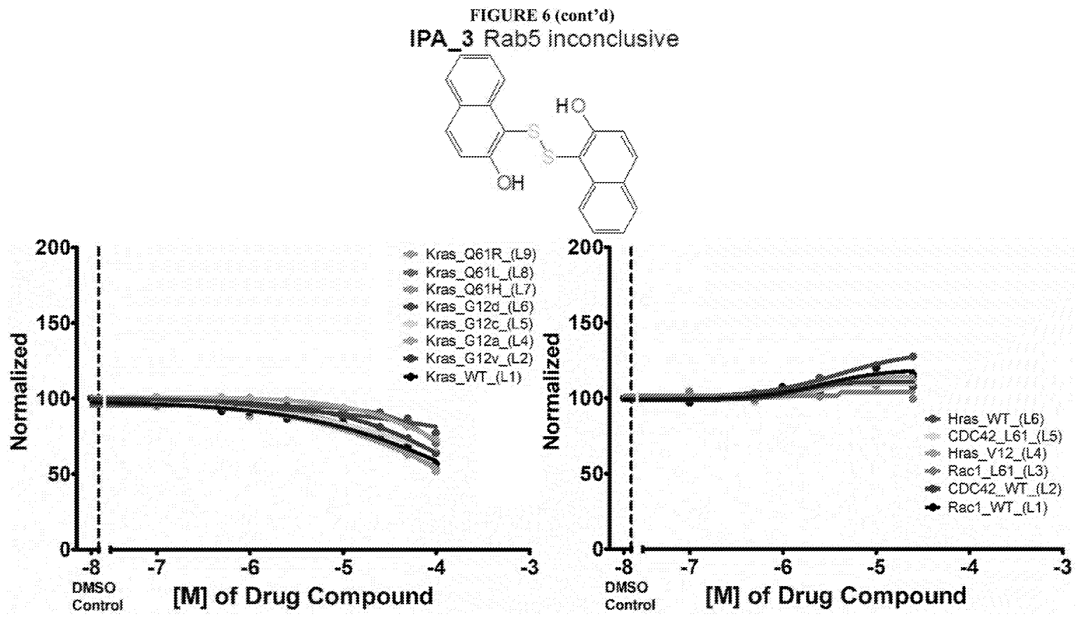

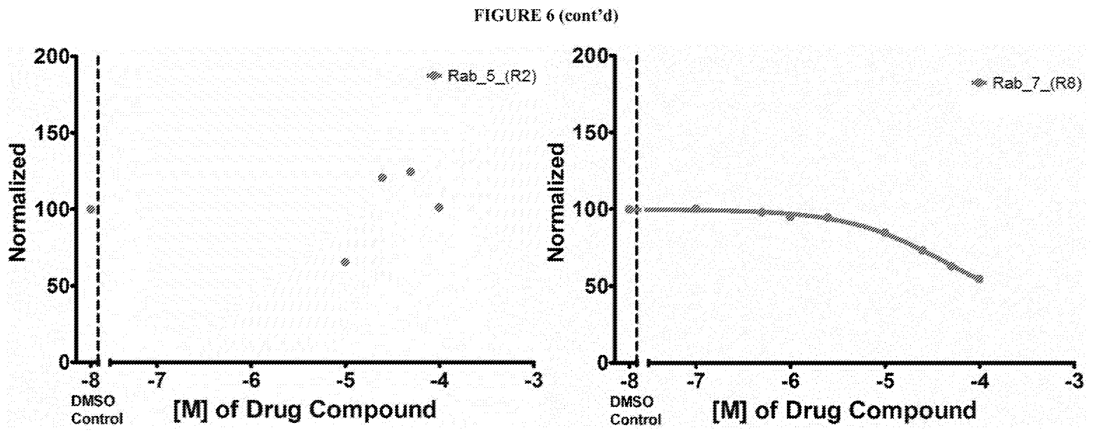

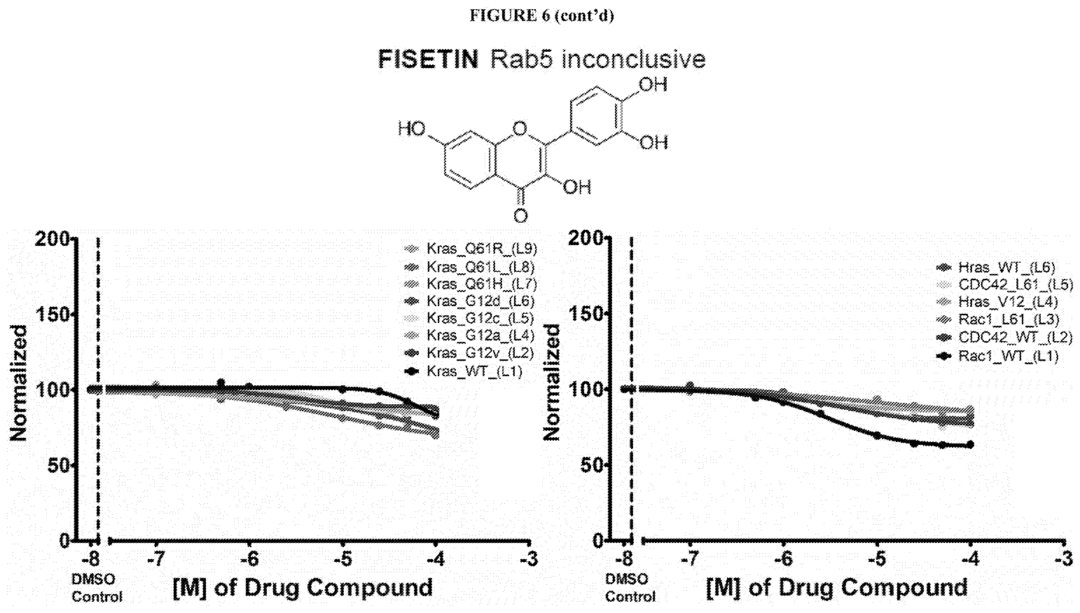

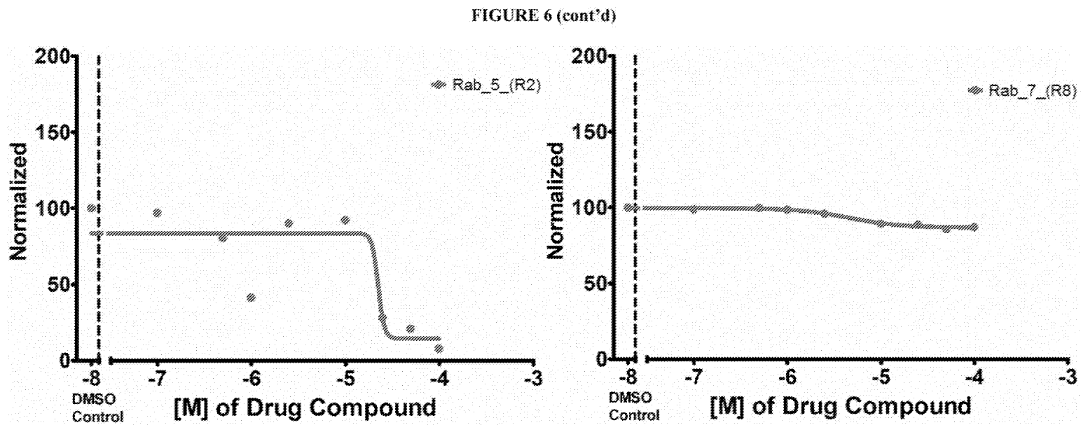

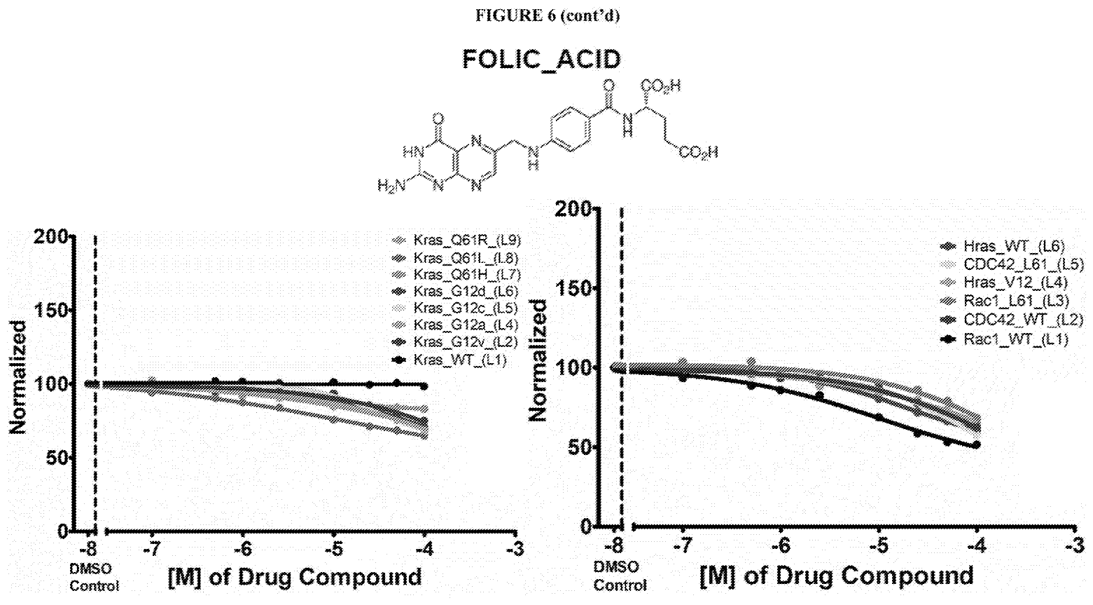

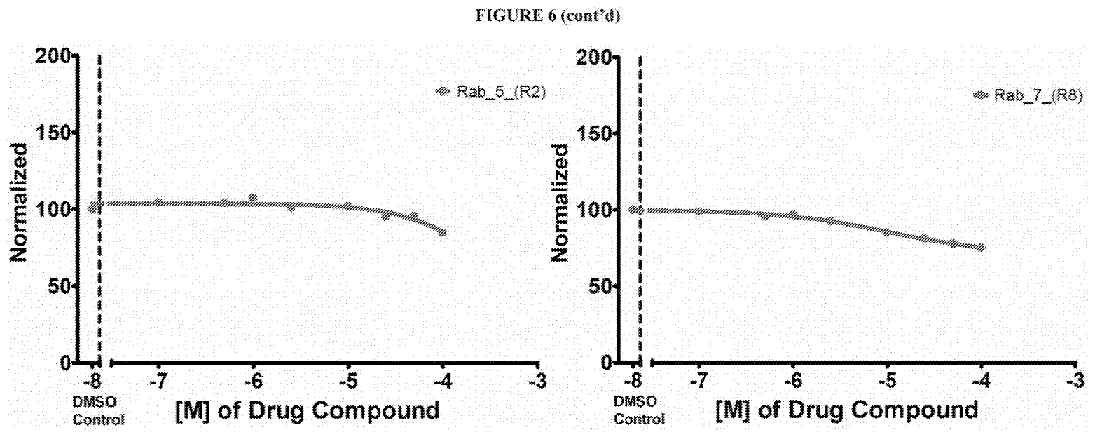

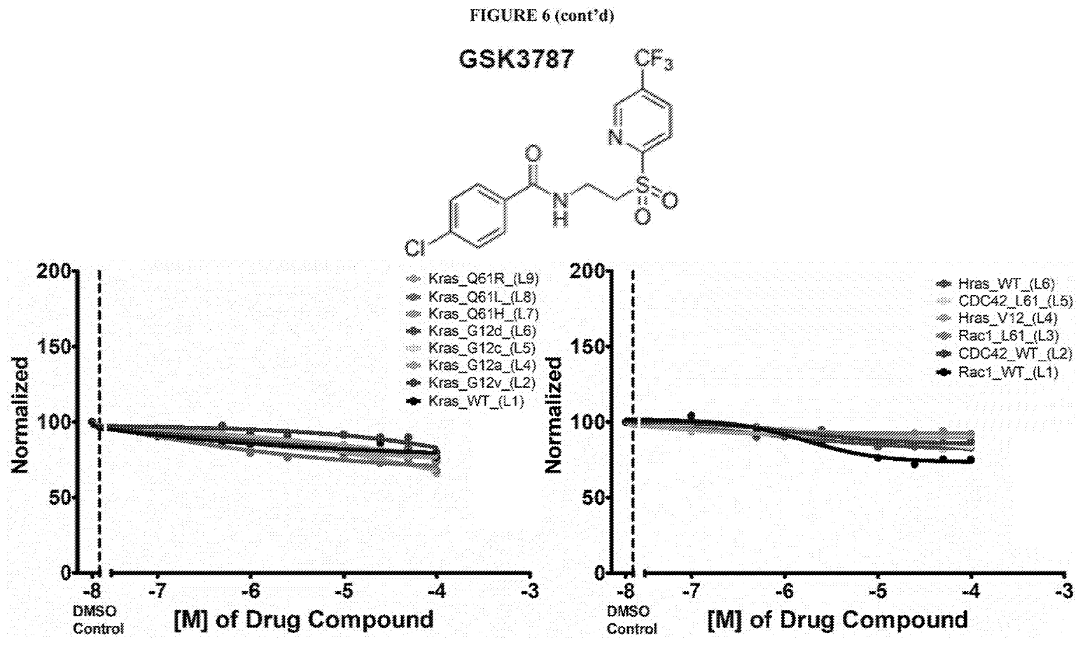

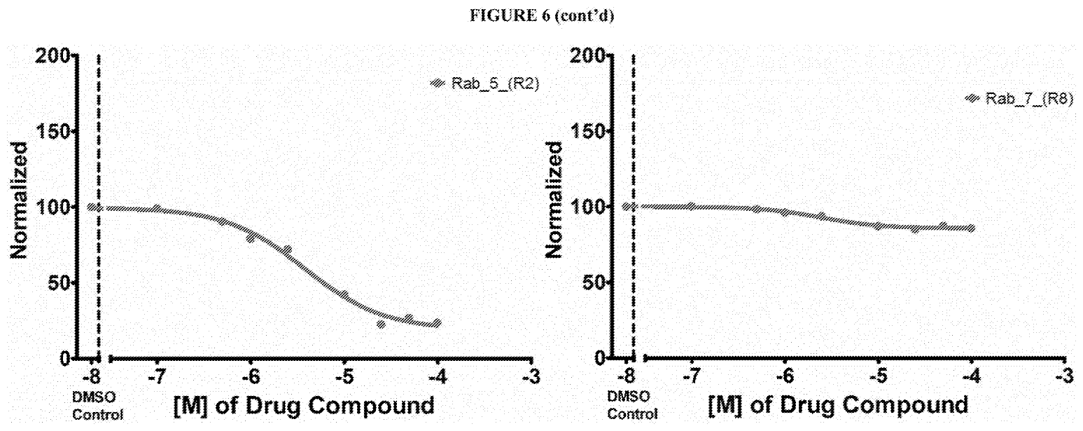

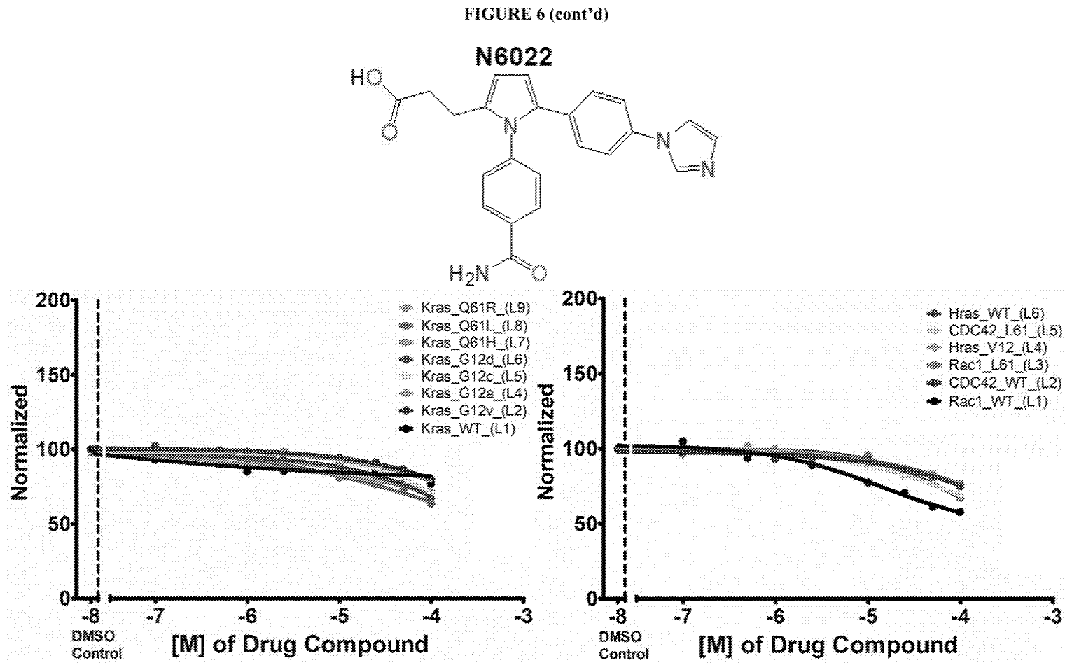

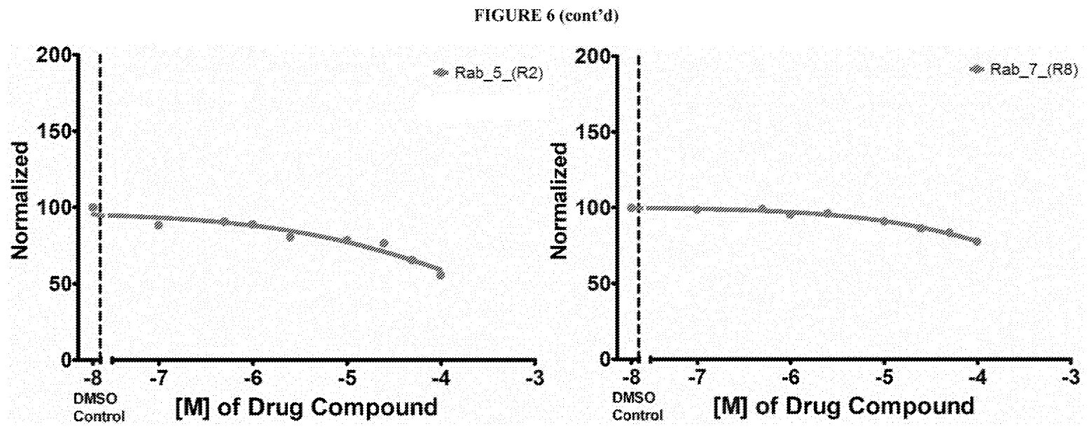

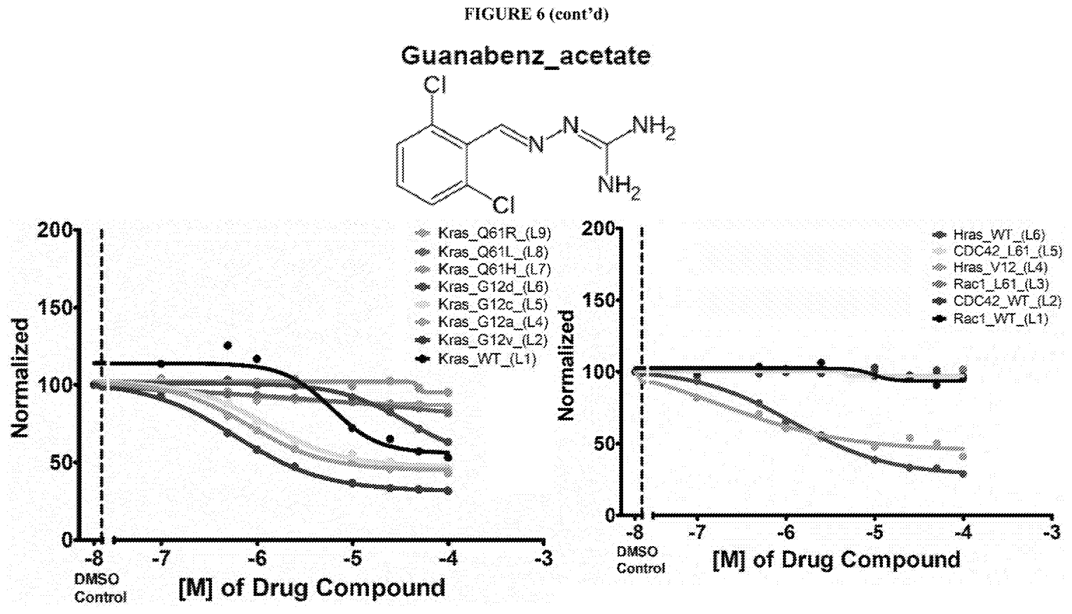

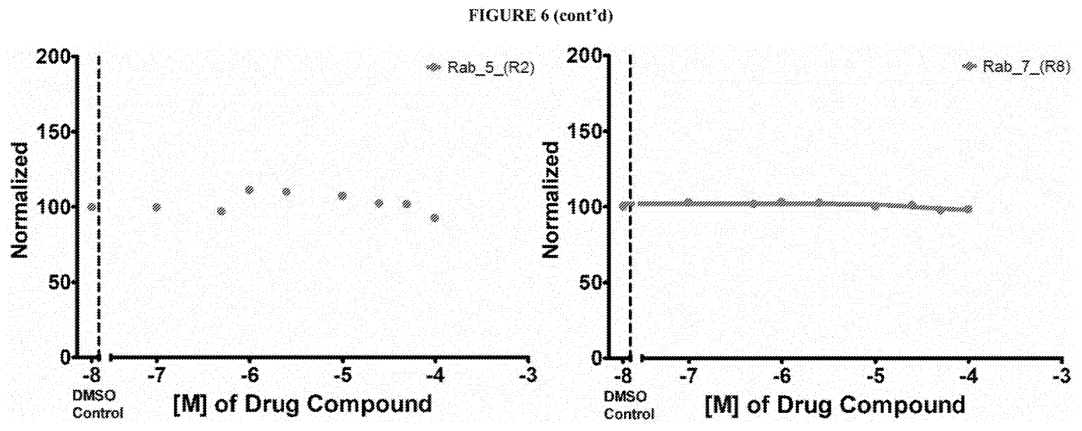

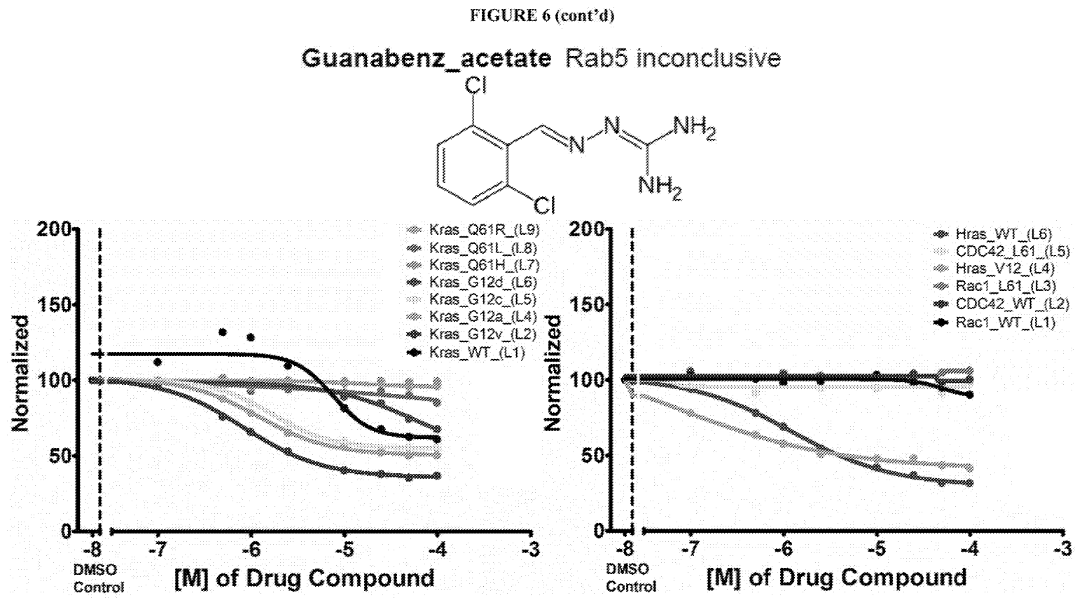

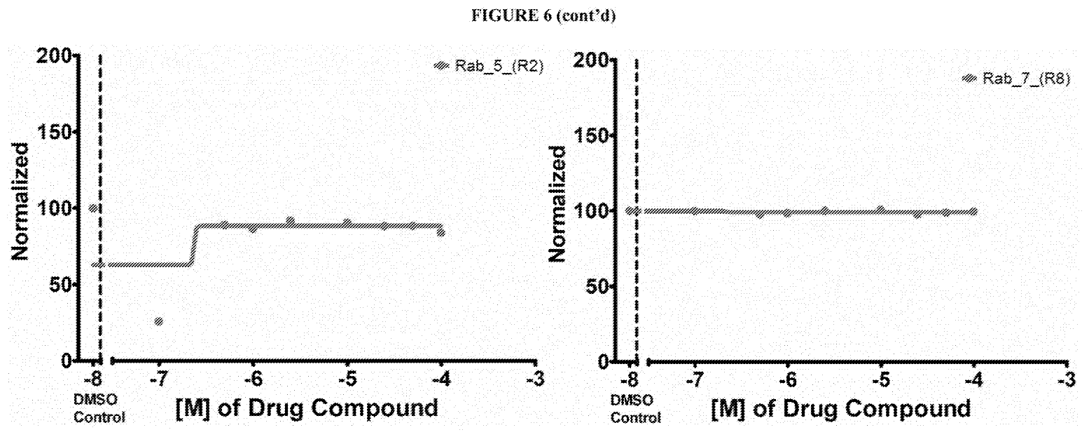

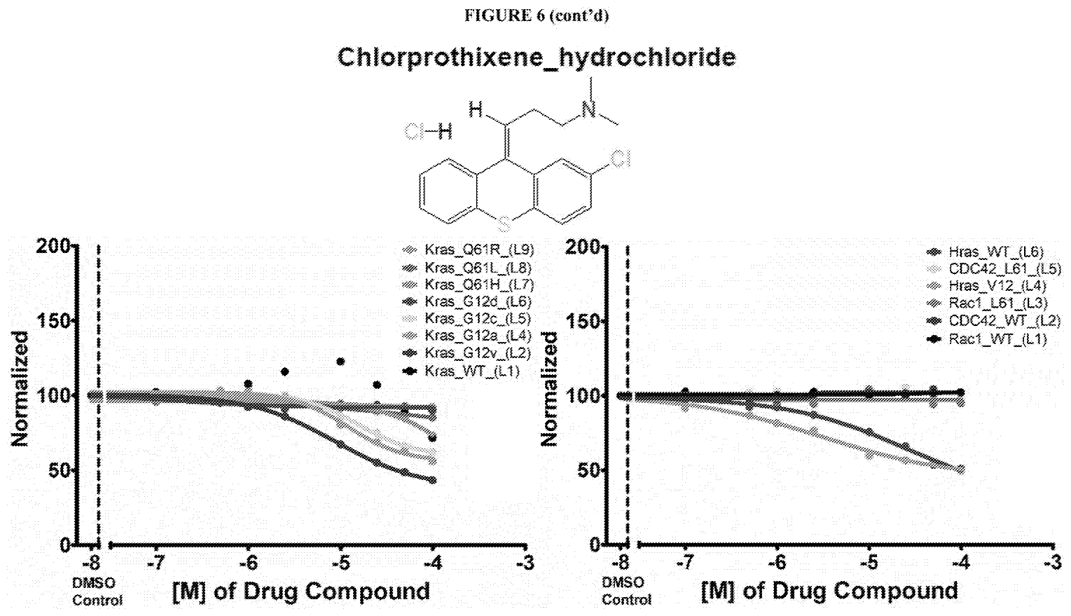

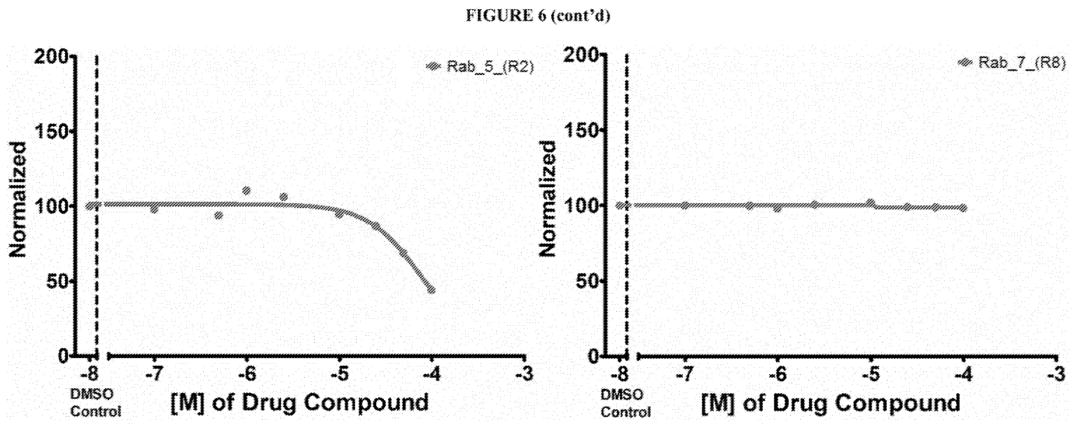

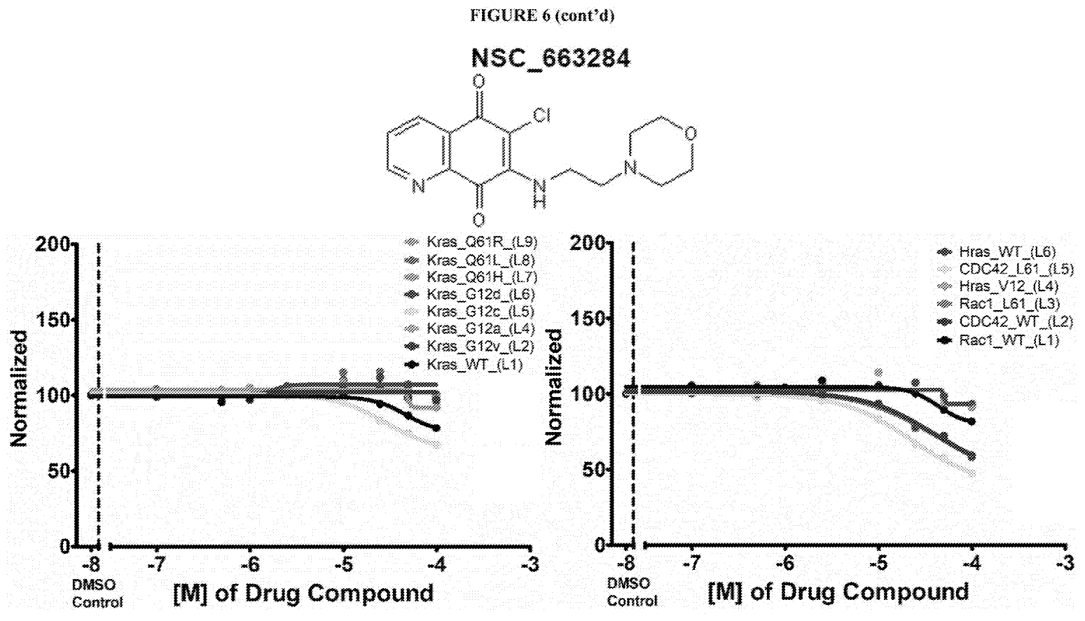

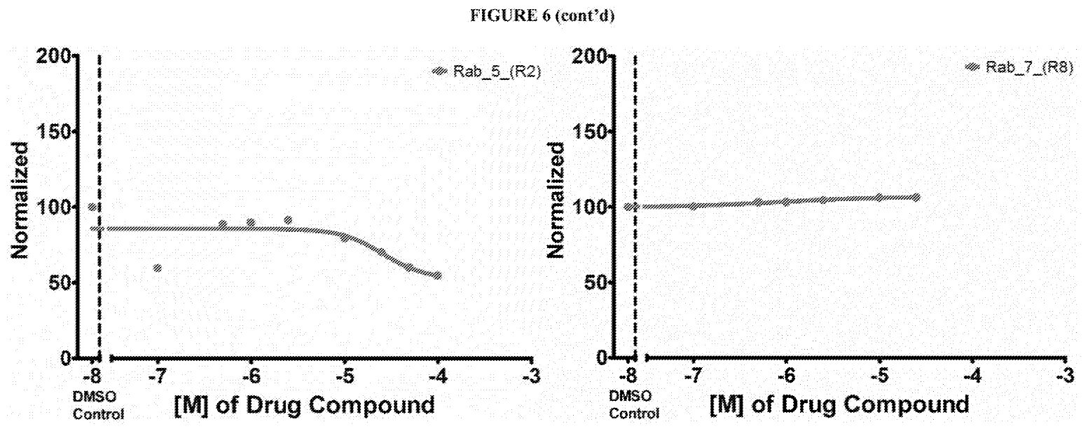

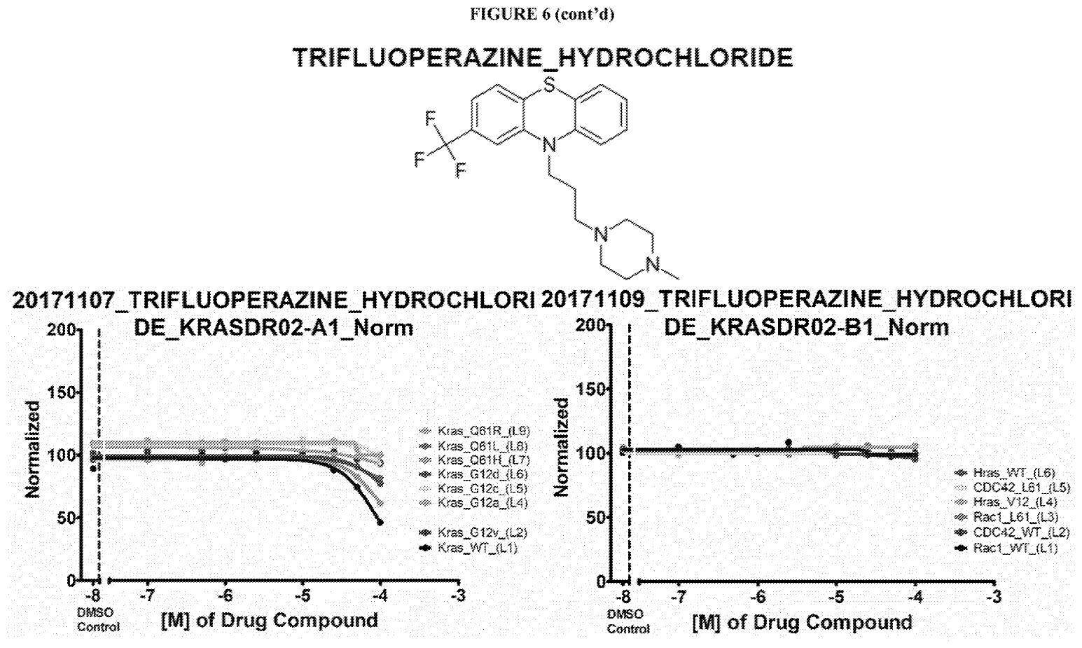

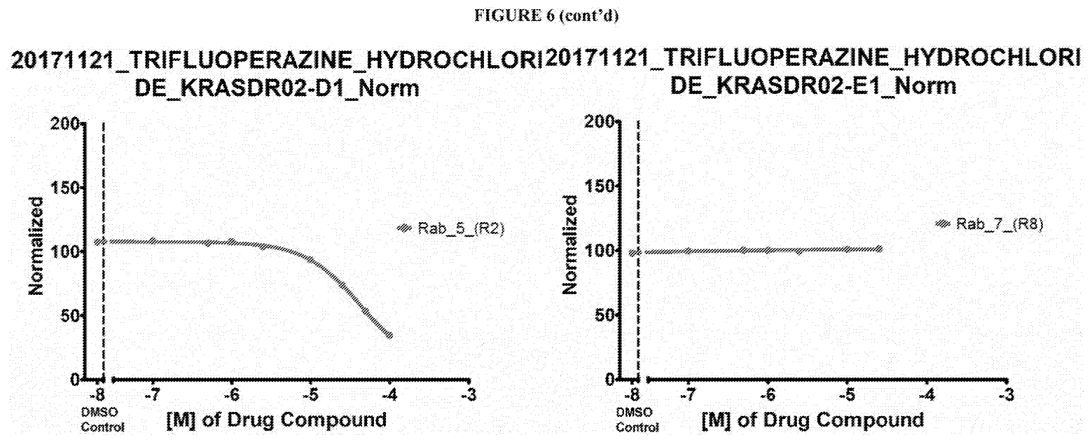

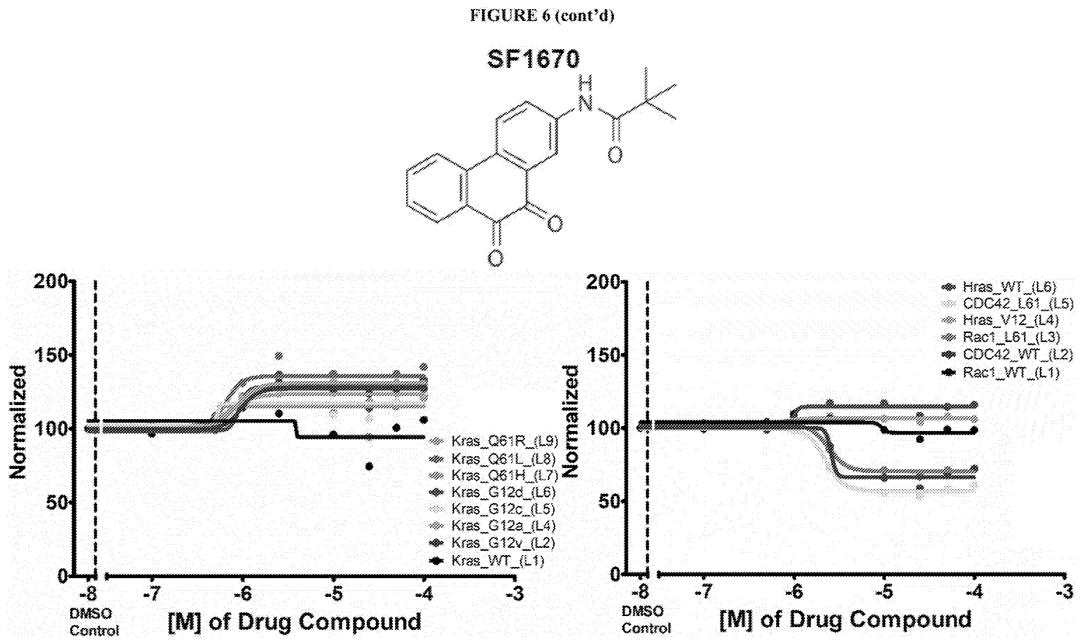

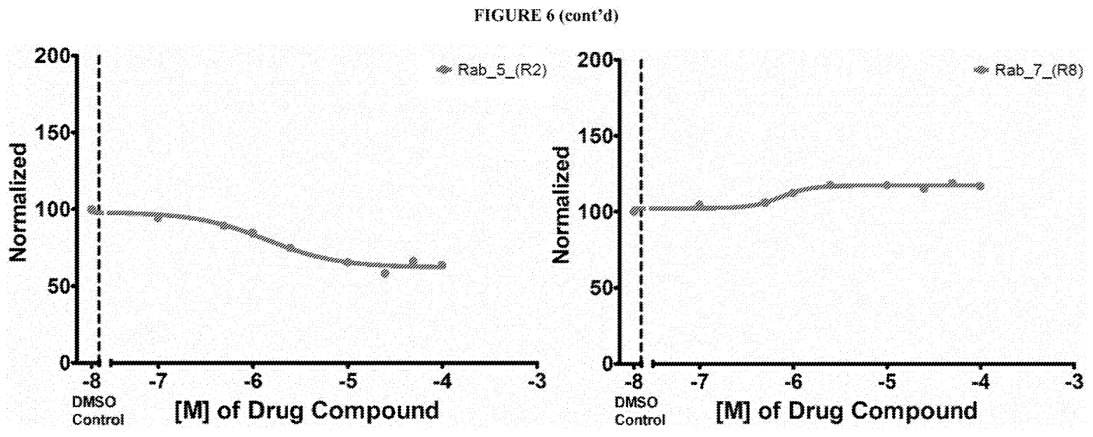

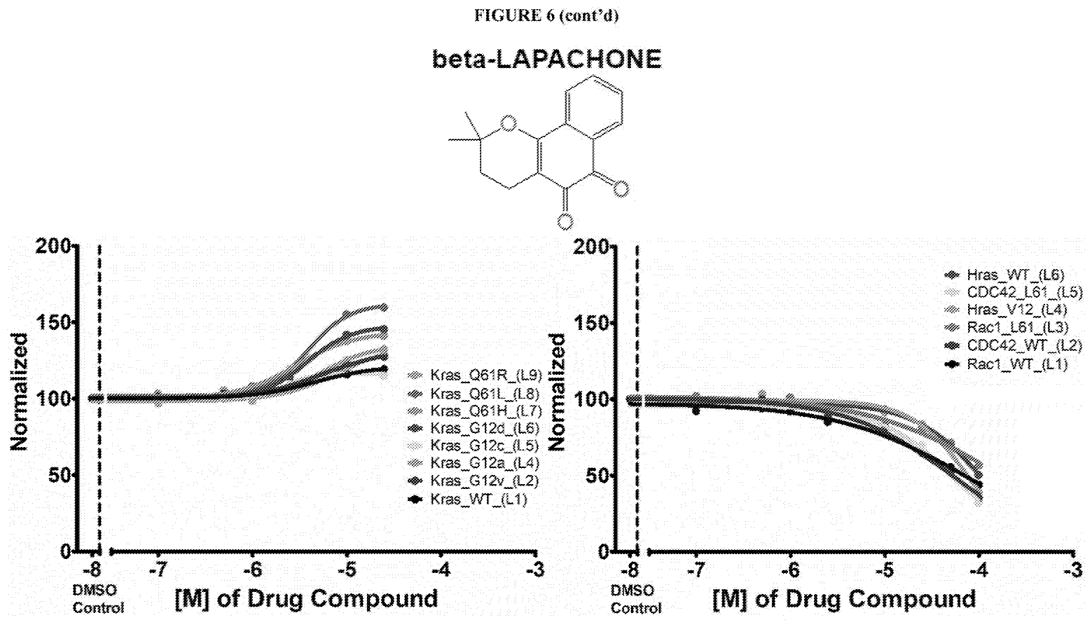

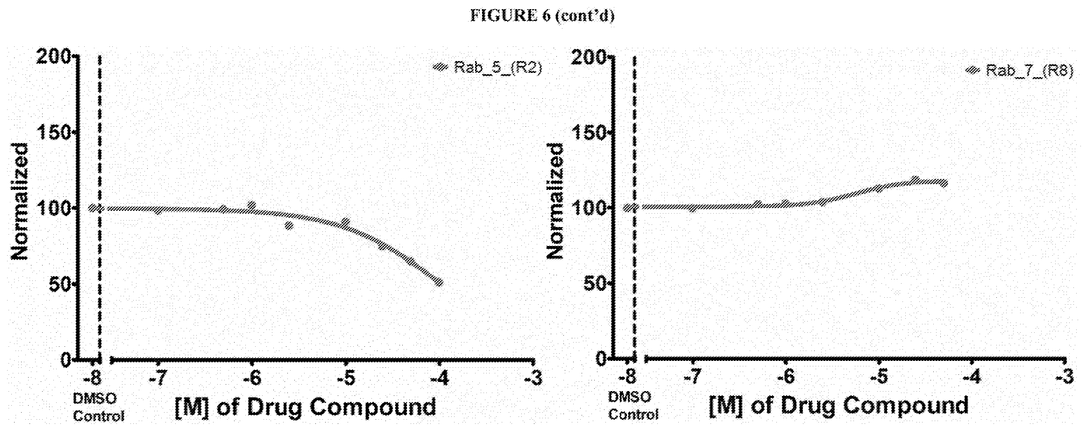

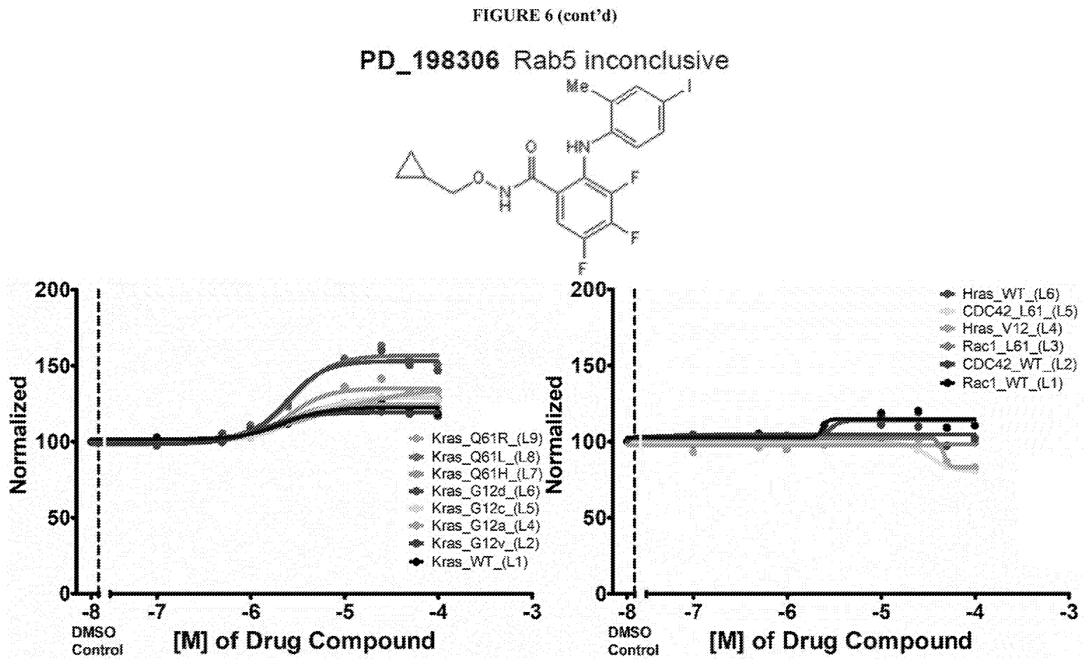

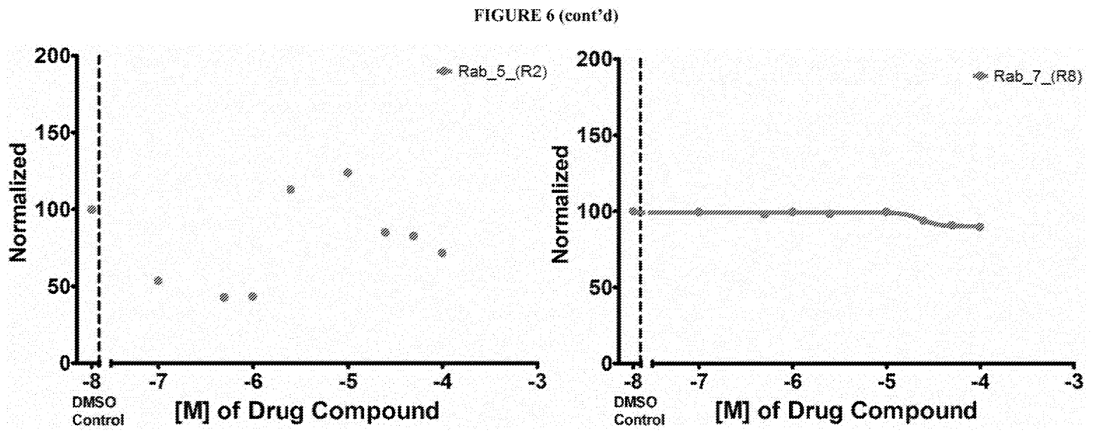

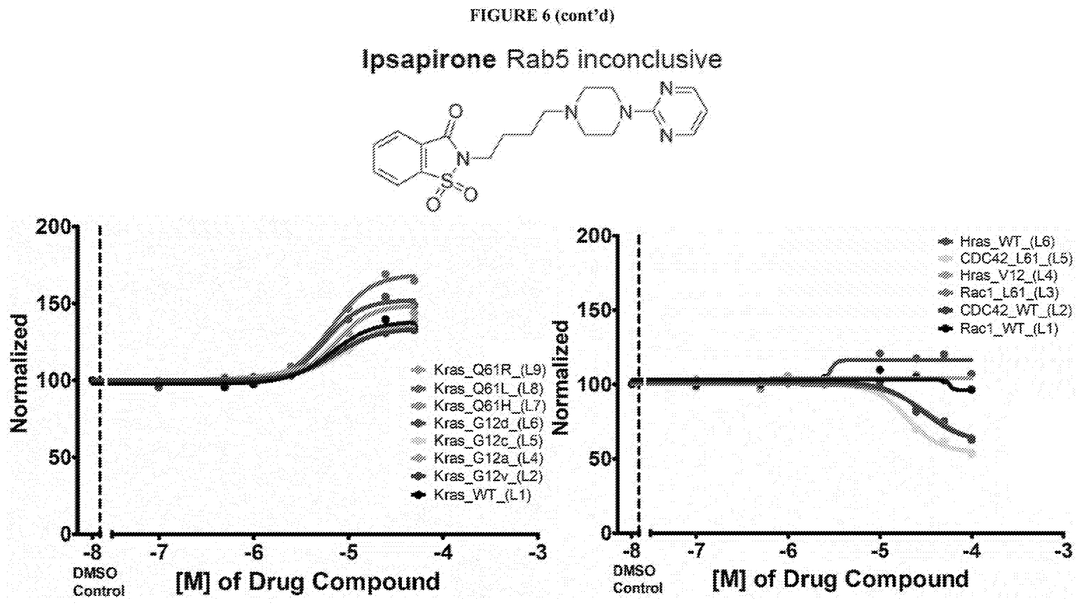

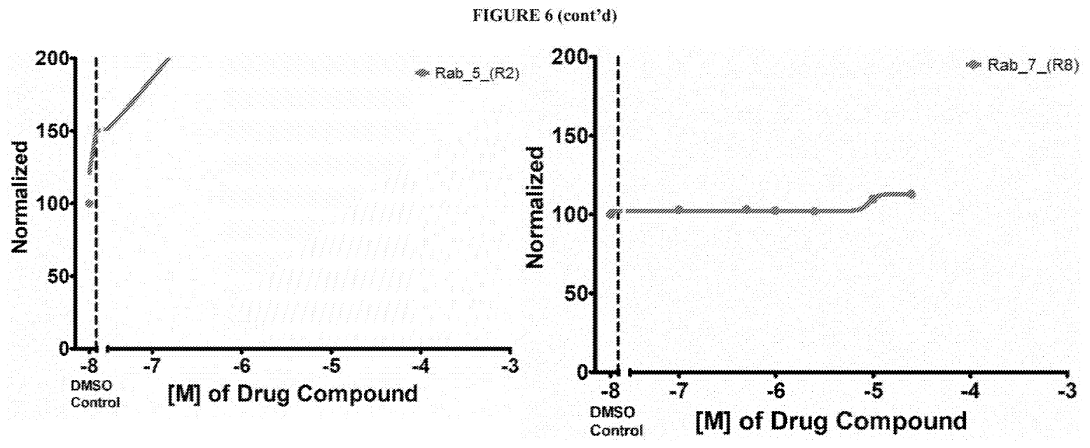

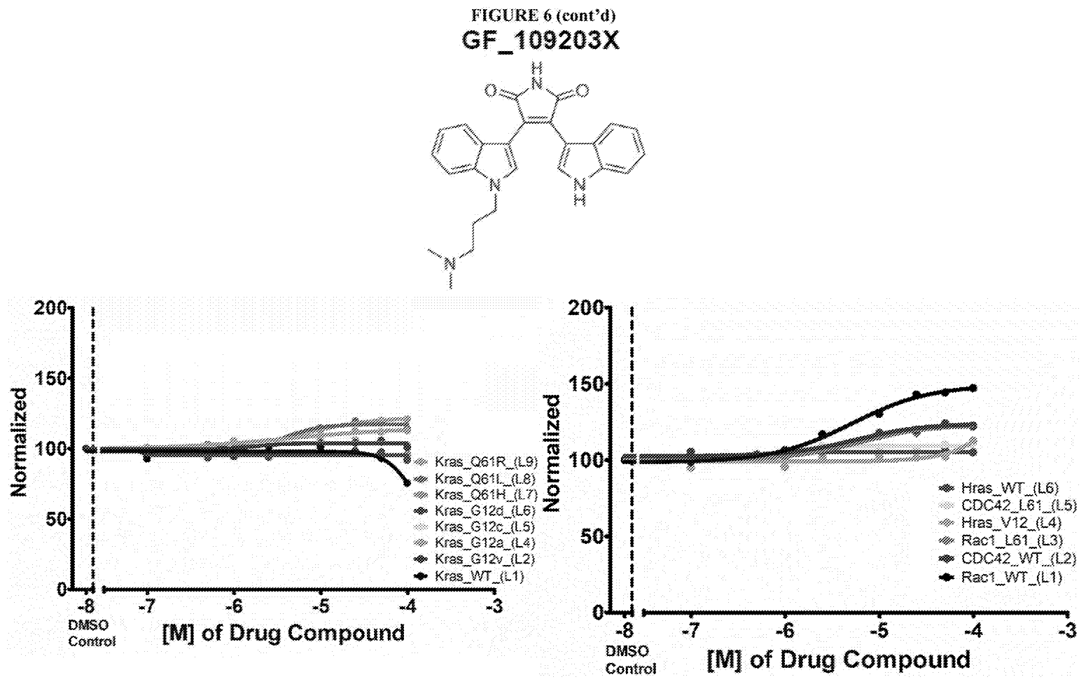

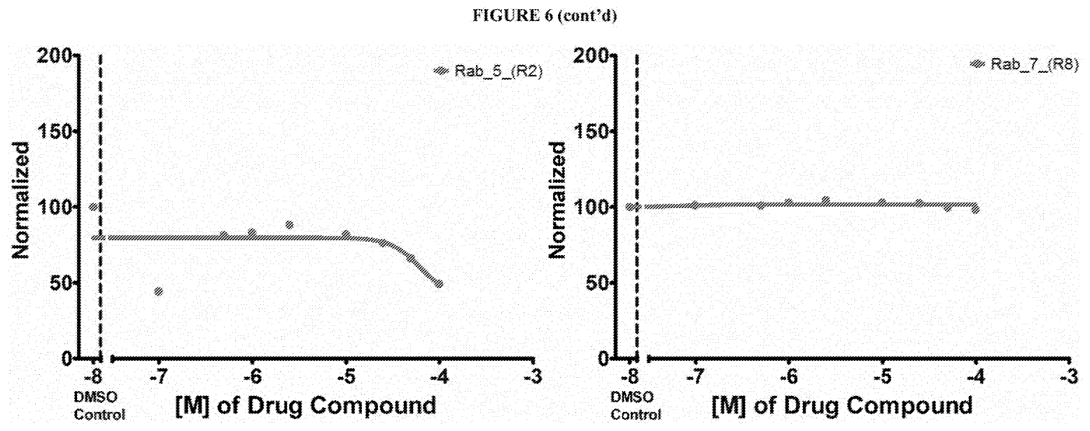

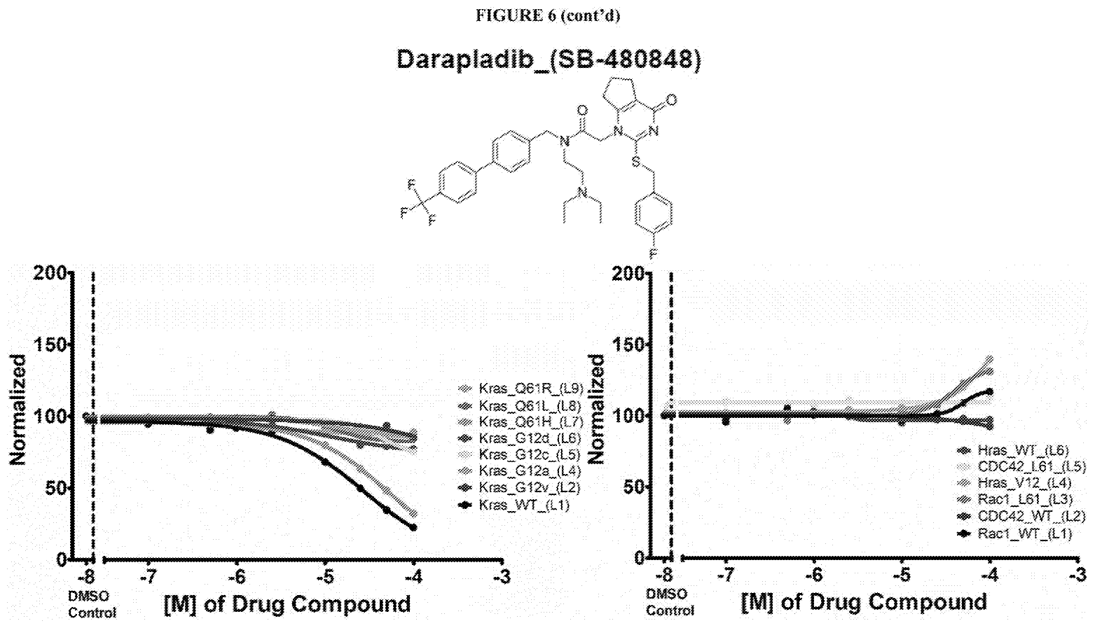

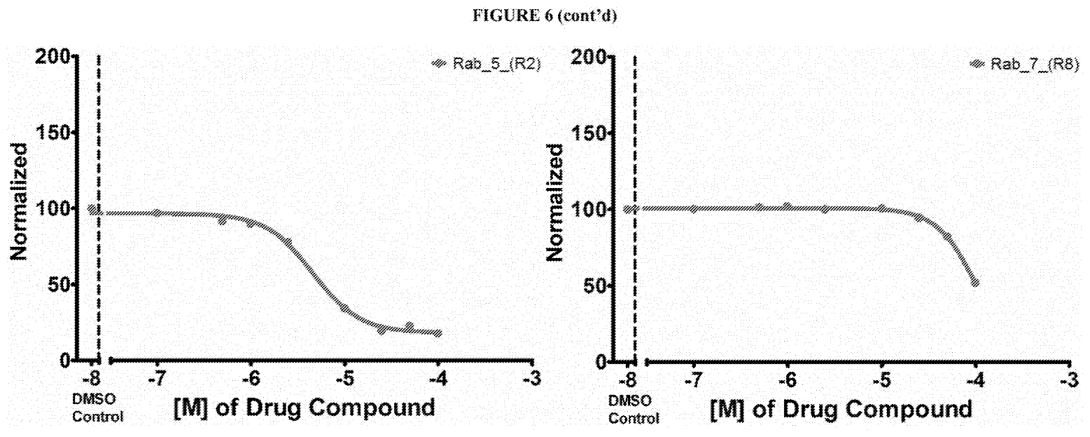

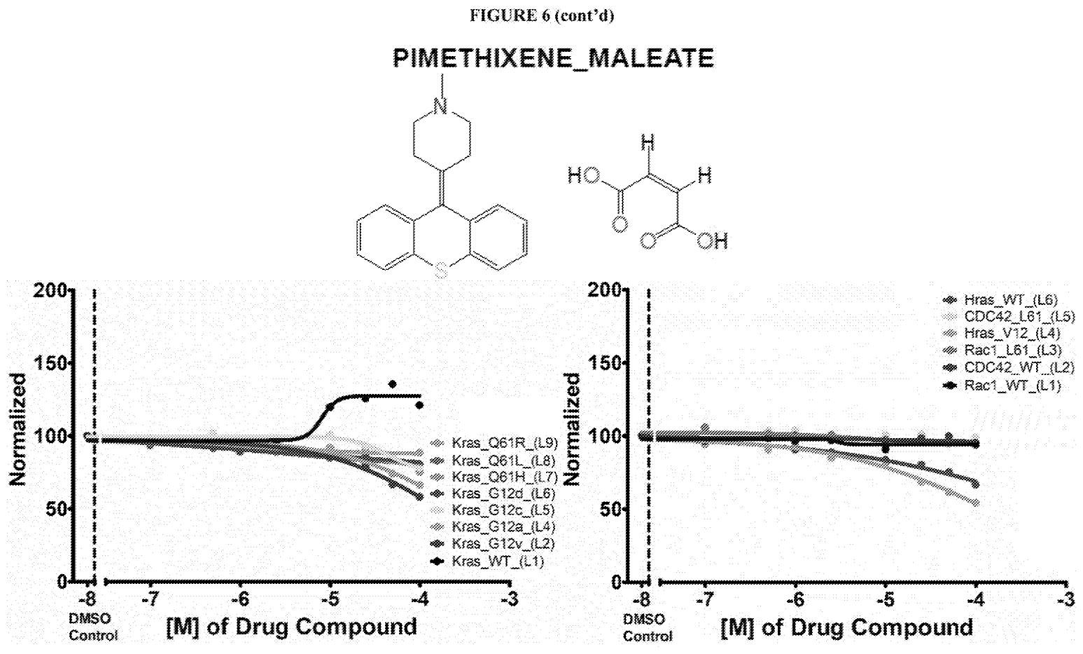

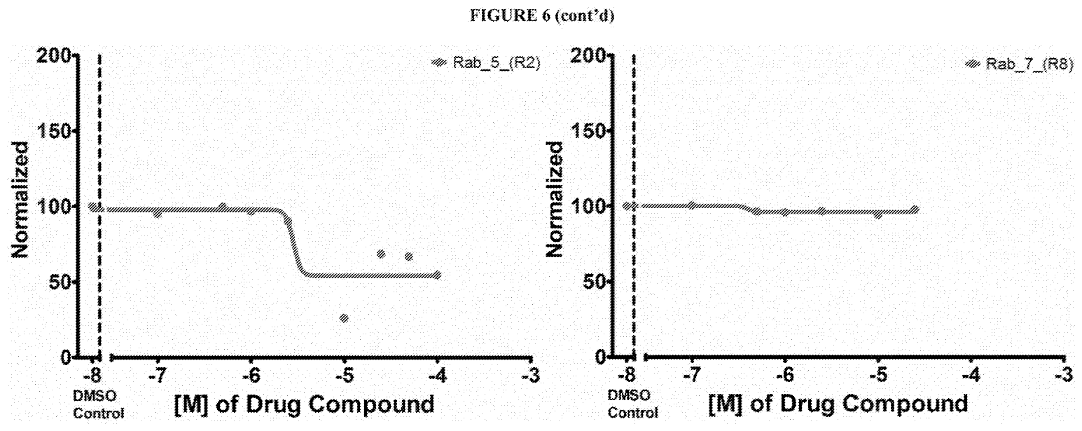

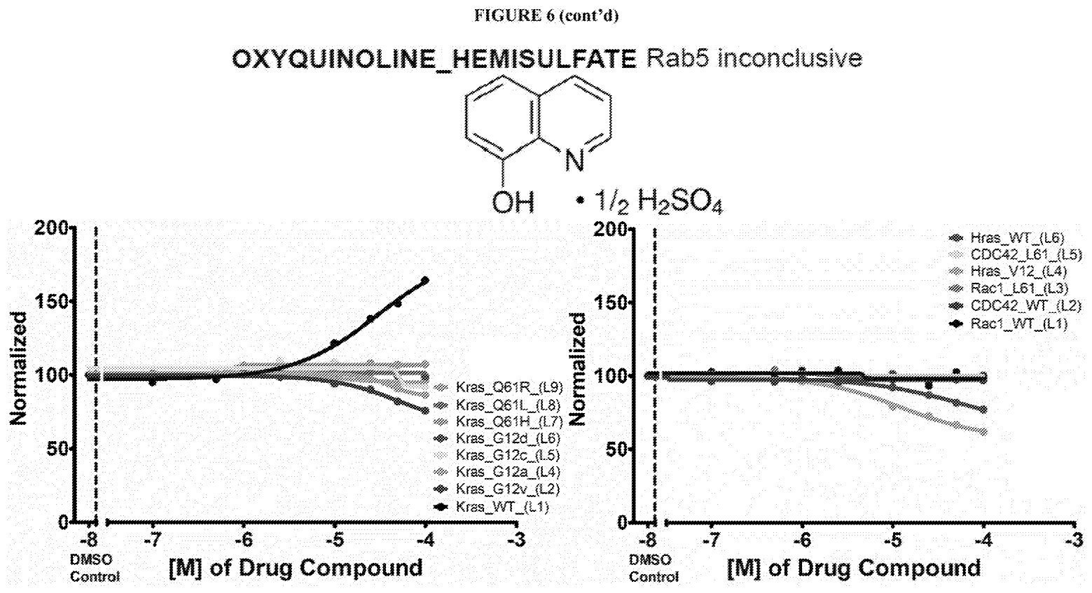

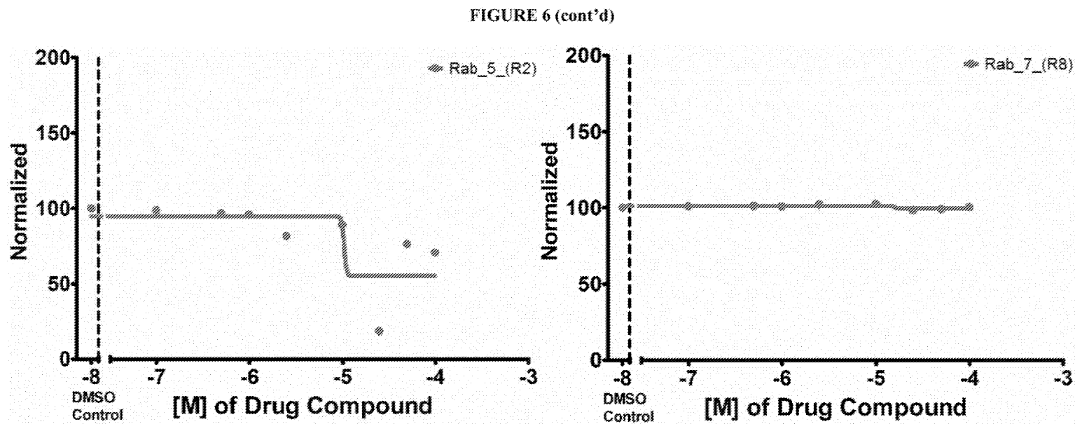

Initial tests were carried out in 50 uL volumes before transitioning to a plate-based, multiplex format where the remaining 8 proteins could be successfully tested. Previous reports determined that the GTPase-coupled glutathione-beads used here express approximately 1.2×10⁶ GST-GTPase molecules/bead⁴² and the amount of beads used represents an approximate concentration of 0.3 nM/GTPase. Examples of dose dependent Bodipy FL-GTP binding in multiplex to the various immobilized GTPase chimeric proteins is shown in FIGS. 1A and 1B. Briefly, 8 (FIG. 1A) or 6 (FIG. 1B) sets of beads, individually coated with GST-GTPase chimeric proteins, were incubated with increasing concentrations of Bodipy FL-GTP for 1 hour at 4° C. Non-specific binding was determined by pre-incubating bead mixtures with 30 μM unlabeled GTP. The nucleotide binding pocket of these GST-GTPase chimeric proteins is likely occupied by GDP, so it is reasonable to assume that the observed binding of Bodipy FL-GTP to the bead-bound GTPases involves an exchange reaction.^{54,61} As expected increasing concentrations of fluorescent ligand leads to increased bead fluorescence with maximal signals occurring between 100-300 nM Bodipy FL-GTP. Data analysis of the separate KRas proteins yielded binding affinities of 1-50 nM which is within the range published previously for these GTP analogues when testing other GTPase family members.^{54,61}. Cumulative affinity calculations from 3-6 binding experiments performed in both single and multiplex formats are given in FIG. 3, Table 1. These data demonstrate stable nucleotide binding over a 1-2 hour time period which would allow for the use of high throughput screening capabilities of commercial sets of small molecules representing 5000 unique compounds.

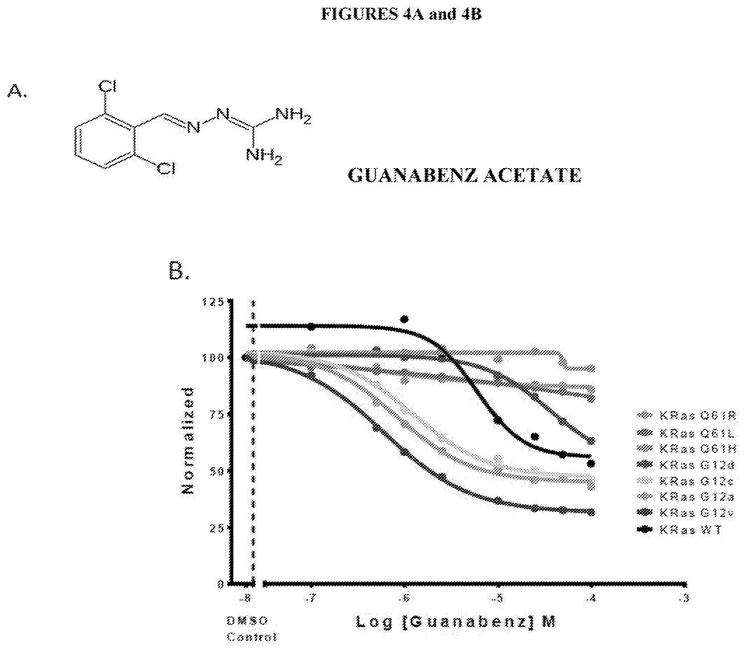

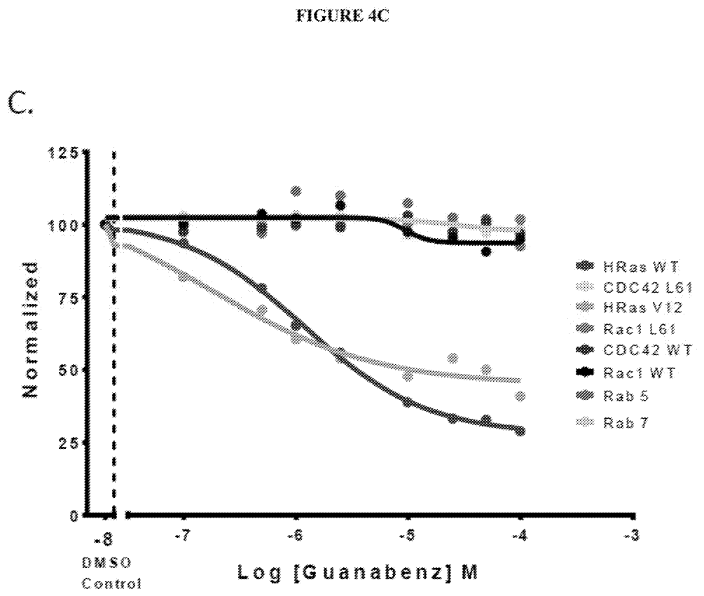

c. Screens. Five commercial libraries comprising greater than 5000 unique compounds that include FDA-approved drugs, natural products, and bioactive small molecules (7073 compounds in total) were screened against 8 KRas proteins. Each well of an assay plate contained 10 sets of beads with variable red fluorescent intensities. Nine protein-coupled bead sets, carrying the different GST-KRas proteins were combined with an uncoupled GSH-bead that serves as a scavenger for GST-proteins that might dissociate during the assay. Bead mixtures were dispensed into individual wells of a 384-well assay plate and incubated with fluorescent GTP in the presence of library compounds. The final concentration of reagents in each assay well was 10 μM compound, 10 nM Bodipy FL-GTP, and 0.1% DMSO. Each bead set was added at 200 beads/μL. Screening statistics and performance are given in FIG. 4, Table 2. Each plate was analyzed both in forward (starting with A1) and reverse direction to account for fluorescent compounds that can carryover during sampling and effect subsequent sample values. The initial hit selection criteria for the primary screen of small molecules was as follows; for any given protein-coupled bead, a hit was defined as a compound well that resulted in a 50% deviation in the Bodipy FL-GTP binding signal compared to the average signal calculated from the DMSO control wells that was also greater than 3 standard deviations from the DMSO controls included on every compound plate. Excess GTP containing wells were evaluated separately and were used, along with the DMSO control wells, to calculate a Z′ value for each bead set. Z′ values serve as an indicator of assay plate reliability.⁵⁸ Over the course of the screen the average Z′⁴⁷ value for each bead set ranged from 0.769-0.880, indicative of a robust assay. Using these selection criteria more than 300 small molecules were chosen for further evaluation. A secondary single point evaluation was performed on these identified primary hits. Identified active compounds were tested at three concentrations (2, 10, and 20 μM). This secondary analysis led to the identification of 61 compounds that were further tested in dose dependent assays.

The KRas multiplex performed in multi-point dose-response confirmed ˜50 KRas modulators (50/5000˜1%). The top 64 compounds were examined for concentration dependent effects on GTP binding using a range chosen for complete dose-response from the cherry pick. The dose response was run as an 8 point multiplex with a well with no compound added for each compound as a control. For visualization of the binding, MFI signals were scaled to 100% binding in the absence of compound.

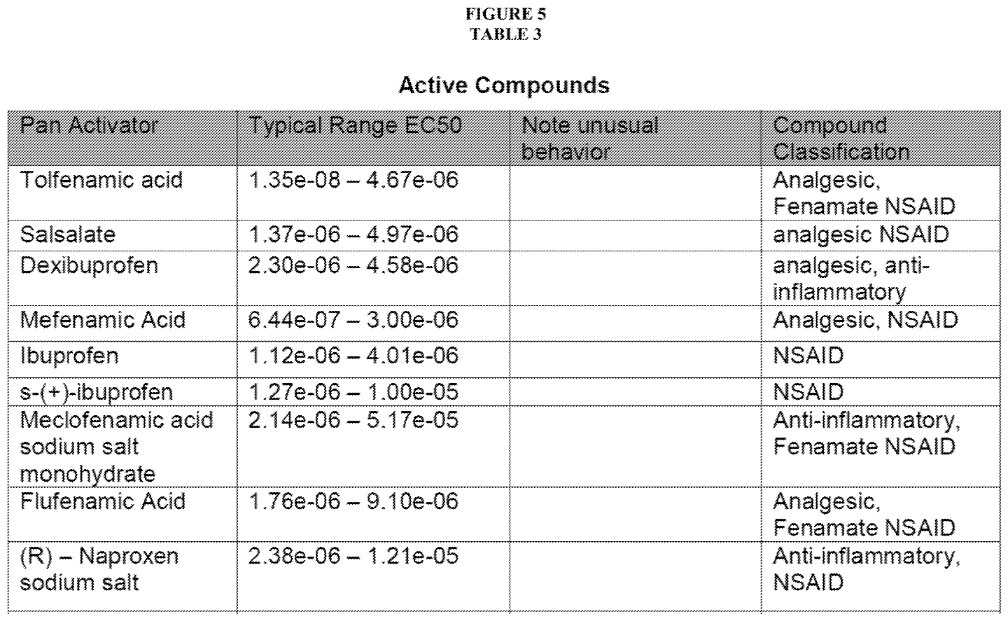

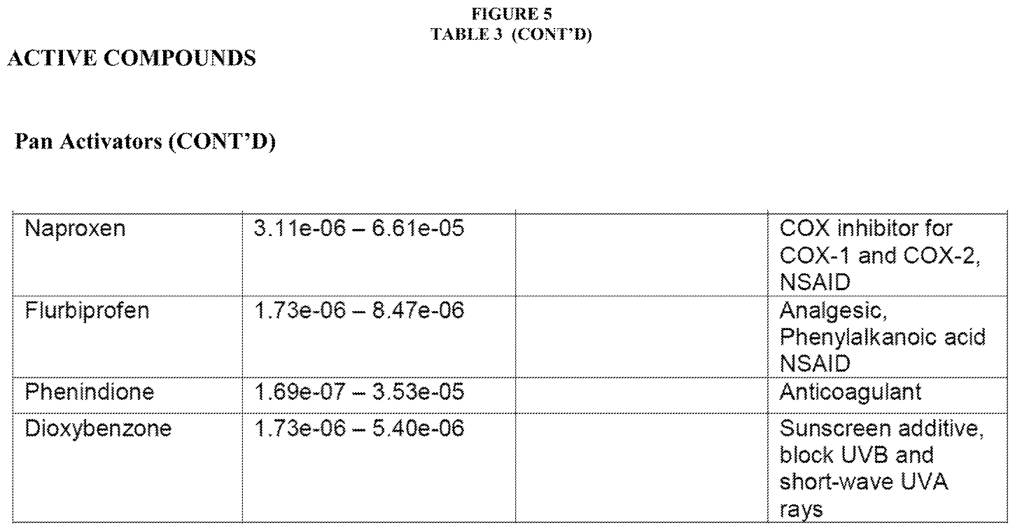

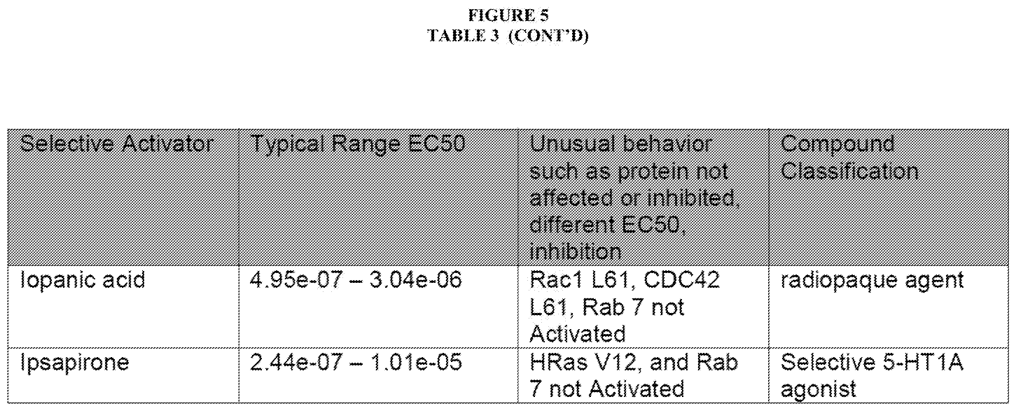

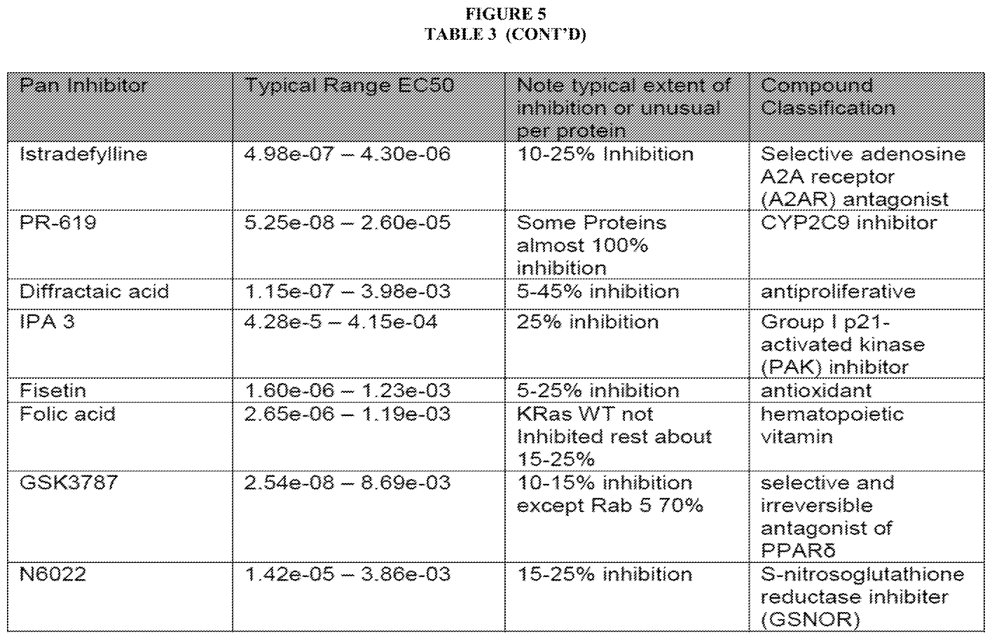

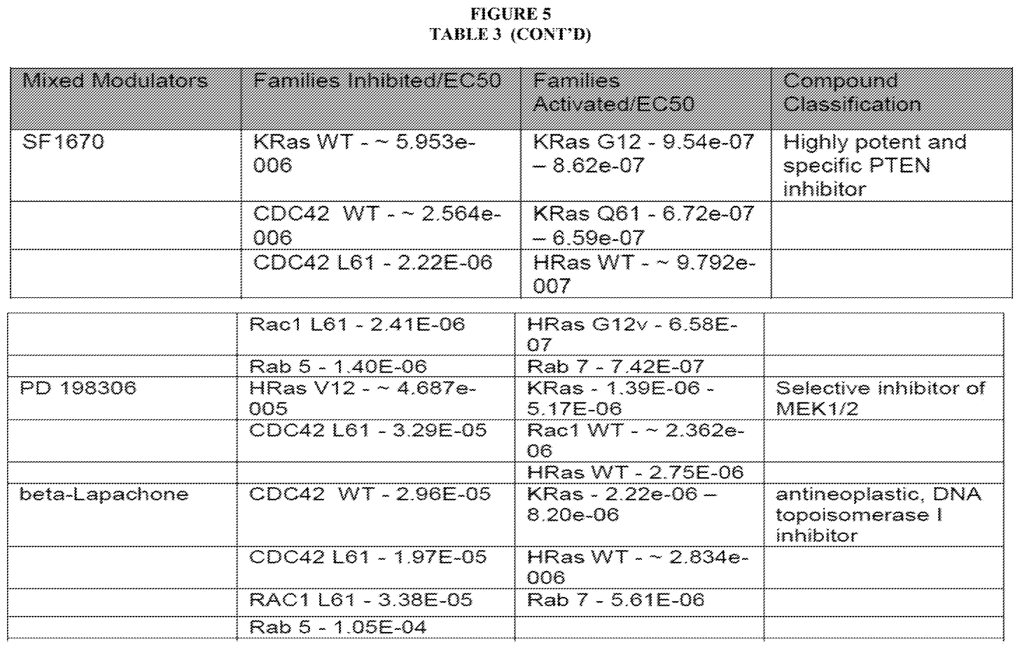

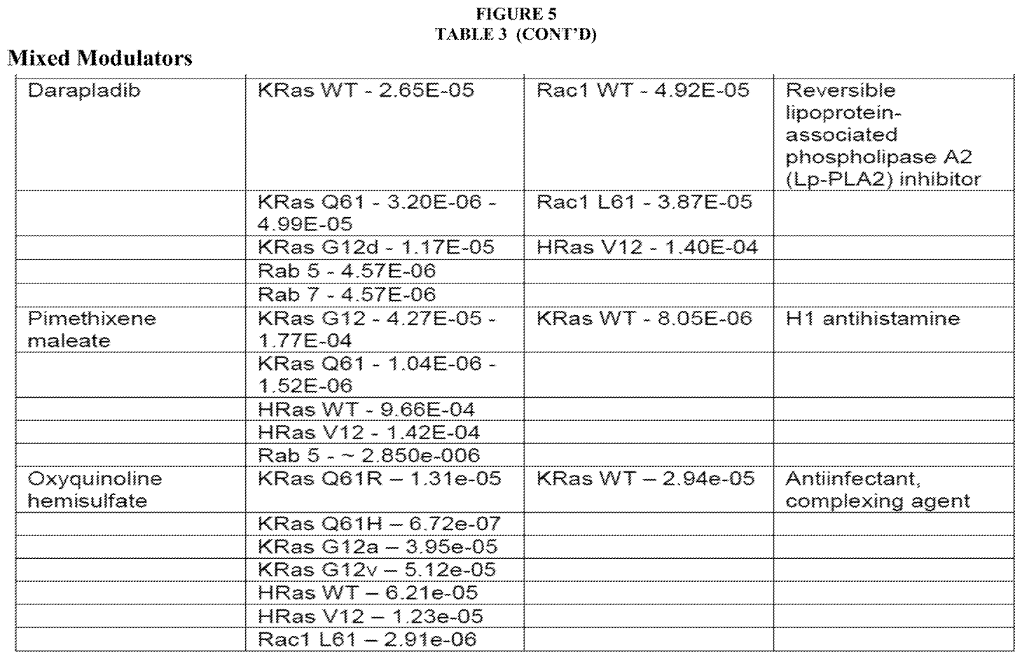

d. Selectivity. To assess selectivity of the compounds with respect to GTPase families we compared the dose-response to the multiplex described previously. Activators showed an increase in the MFI for proteins while inhibitors showed a decrease in the MFI for the proteins. For analysis, all data was normalized to 100% using the vehicle control (DMSO) wells. Compounds were identified as Activators, Pan Inhibitors, Selective Inhibitors, and Mixed Activity Modulators as shown in FIG. 5, Table 3 and FIG. 6.

Pan activators increased the binding of BODIPY-FL GTP to essentially all of the GTPases tested. They include NSAIDS, as previously reported⁴⁷. In approximate rank order, these include: tolfenamic acid, salsalate dexibuprofen, mefenamic acid, Ibuprofen, s-(+)-ibuprofen, meclofenamic acid sodium salt monohydrate, fufenamic acid, (R)-naproxen sodium salt, naproxen, and fluribuprofen. It is worth noting the variation in the binding increase. Because a single concentration of the BODIPY-FL GTP was used, and the EC50 varies among proteins (Table 1), the increased binding is larger when the concentration of BODIPY-FL GTP is lower (i.e., lower fractional occupancy). It is worth noting that (R)-naproxen sodium salt showed greater activity than did the naproxen sample. This may be due to naproxen having both R and S enantiomers while the (R)-naproxen sodium salt only has the R enantiomer. Similarly, S-ibuprofen was lower affinity than the mixture.

The inventors had previously identified “canonical” activator probes^{35,48} with two aromatic rings, one carboxylate, and a bridging chain, analogous to fenamates (flufenamic acid, melcfenamics acid, mefanamic acid, and tolfenamic acid), as distinct from the propionic acid NSAIDS (naproxen, ibuprofen, and dexiprofen but not flurbiprofen). A recent report identified bis-phenols as activators with ˜1000 fold less potency than the most active of those we have described.⁶³ It is interesting that the fenamate PD198306 appears to show mixed activity with mostly activation, but some inhibition as well. The NSAID ketorolac and NSAID-like sulindac sulfide have been reported as inhibitors.^{47,64} In addition, the NSAID-like iopanic acid (radiocontrast agent) and the aromatic phenidone and dioxybenzone, with acidic PKa, were also activators. In contrast, the orthoquinones B-lapachone and SF1760 with acid PKa exhibited mixed activity (see Mixed modulators below).

Pan inhibitors that decrease the binding of BODIPY-FL GTP to GTPases include: Istradefylline, PR-619, Diffractaic Acid, IPA 3, Fisetin, Folic acid, GSK3787 (HRas), N6022, and NSC 663284. The comparison of Kᵢ and Cₘₐₓ suggests that istradefylline and the polyphenolic coloring agent fisetin (3.5×10⁻⁵) could have physiological activity. The structural relationships among these molecules, our pan inhibitor ML282, and those previously described⁵ are worthy of further study. The physiological relationship between the extent of inhibition, the mechanism, and cell physiology also remain to be studied.

Selective inhibitors decrease the binding of BODIPY-FL GTP to a subset of the proteins. The most active include guanabenz acetate, an antihypertensive α2 adrenergic agonist, and chlorprothixene hydrochloride, an antiemetic as compared to NSC663284 and trifluoperazine. Guanabenz acetate and chlorprothixene hydrochloride appear to be selective for KRas WT, the KRas G12 mutants, and the HRas proteins. Based on Cₘₐₓ and K_{I}, both drugs have the potential for in vivo physiological activity^{65,66}. Our earlier screening previously identified a CDC42 selective inhibitor characterized in some detail, and a RHO family selective inhibitor.^{42,45} Guanabenz, based on its upstream activity against Elf4 as a regulator of Rac1 has been of interest in a recently closed bone resorption/metastasis trial. The tricyclic antidepressant chlorprothixene is related to a series of molecules identified by Burns et al as selective for Ras/SOS and similar to spiclomazine.

Moreover, for the first time to our knowledge, our screens identified a number of modulators with mixed activity, including the orthoquinolones SF1670 (activates Q61 KRas mutants) and Beta-lapachone mentioned above, the fenamate-like PD198306, ipsapirone (selective 5-HT1a agonist), GF109203X, darapladib, pimethixene maleate, and oxyquionline hemisulfate. It is notable that as a class the trifluorperazine/tricyclic antidepressants exhibit weak inhibition (trifluoperazine), selective inhibition (chlorproxithene), and mixed activity (pimethixine maleate). A potential role of maleate as a divalent chelator has not been further investigated with respect to GTPase.

Overall, the following observations are worthy of further consideration: 1) a structural progression from pan to selective inhibitors within small molecule chemotypes; 2) the potential role of the aromatic acids as modulators of divalent cation sites with respect to nucleotide binding; 3) inhibition of nucleotide binding by sulindac sulfide and ketorolac; 4) the potential for a single drug to exhibit mixed activity for GTPases through allosteric divalent cation site modulation.

Mechanism of Action.

To determine mechanism of action, we evaluated binding and dissociation of Bodipy FL-GTP in real-time kinetic experiments in the presence of Guanabenz acetate or GTP. Tests included pre-binding of Bodipy-FL GTP at 4′ C. or RT in the presence of Mg²⁺. In general, the compound was not able to displace Bodipy FL-GTP whereas it could be displaced by GTP. Since the KRas mutants were not stable in the presence of Mg, the inventors also tested stable GTPases in the absence of Mg, in which case GB also did not induce dissociation.

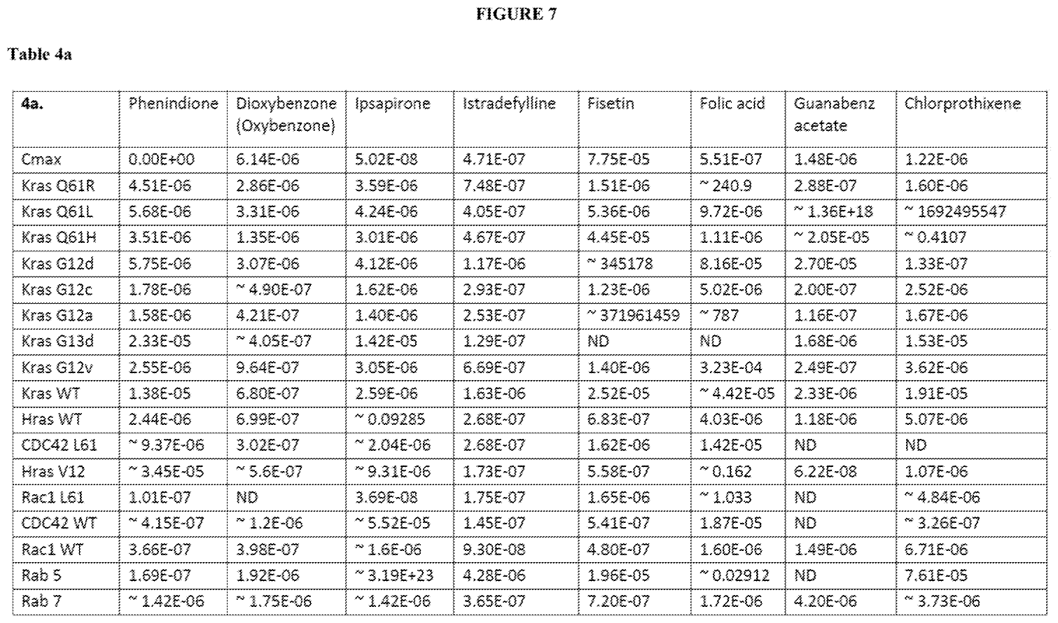

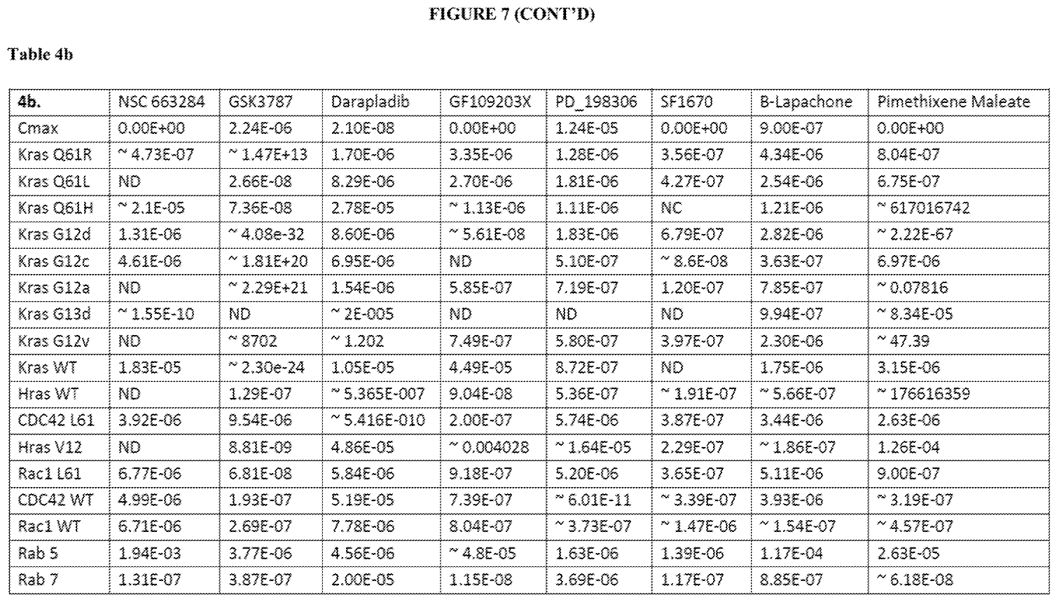

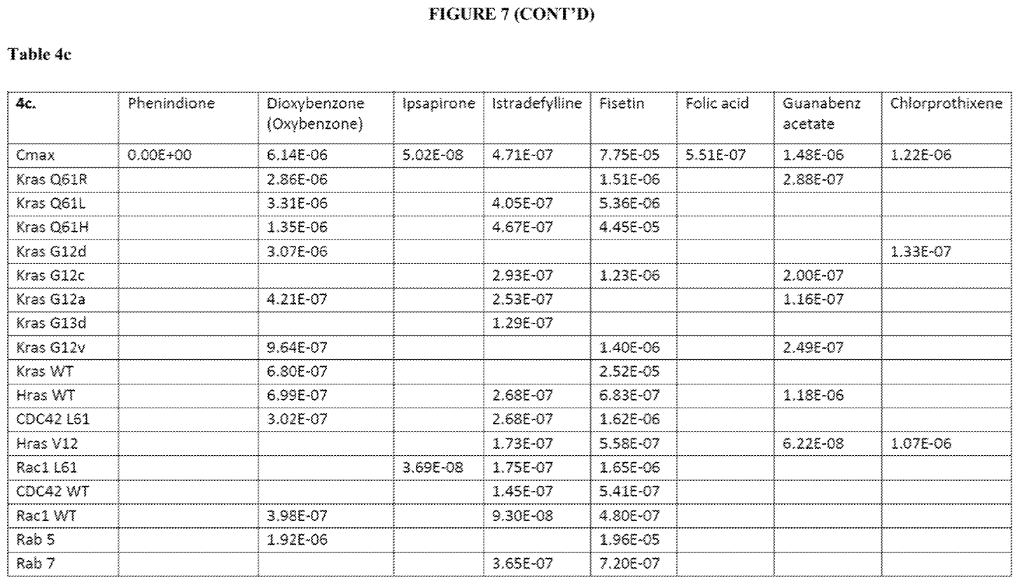

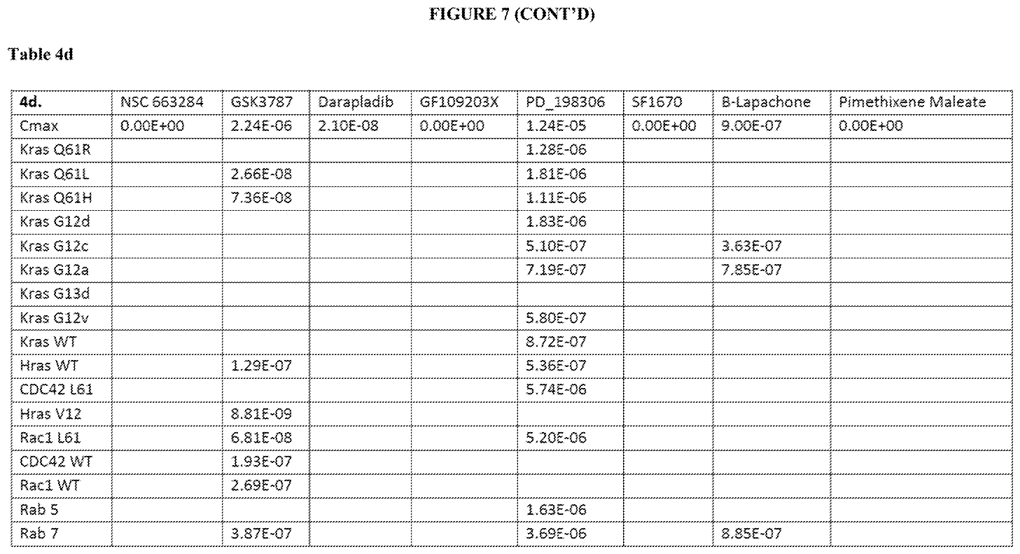

It was then elected to perform association rate analysis, using the order of addition of reagents used in screening, where guanabenz was added first, incubated at 4° C., then Bodipy FL-GTP was added at room temperature. Dramatic differences in association were noted between the GTPases identified previously as selective for guanabenz action (KRas G12v and HRas G12V vs Kras Q61R, Rac1 L61 and CD C42) (FIG. 7).

Discussion

Three RAS genes (HRAS, KRAS and NRAS) comprise the most frequently mutated oncogene family in cancer. With single point mutations (99%) predominately localized to codons 12, 13 and 61 (FIG. 9). A common feature of these point mutations is that they render Ras insensitive to down regulation by GTPase activating proteins (GAPs) that catalyze hydrolysis of GTP, resulting in constitutive signaling.⁶² As such, individual mutants have historically been considered oncogenic equivalents. However, recent observations suggest that codon-specific missense mutations result in mutant Ras proteins with different biochemical and tumorigenic properties that exhibit varying abilities to engage signaling effectors.^{5,62} Differences have also been observed in response and resistance to specific anti-cancer therapies. Delineating these differences has important clinical and biological implications. In particular, KRAS mutations are most prevalent in pancreas (G12D) followed by colon and lung (G12C).^{5,7}

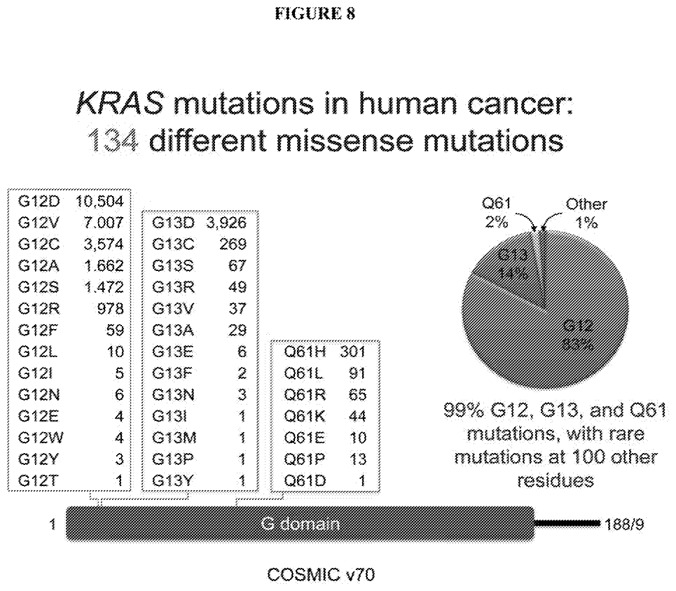

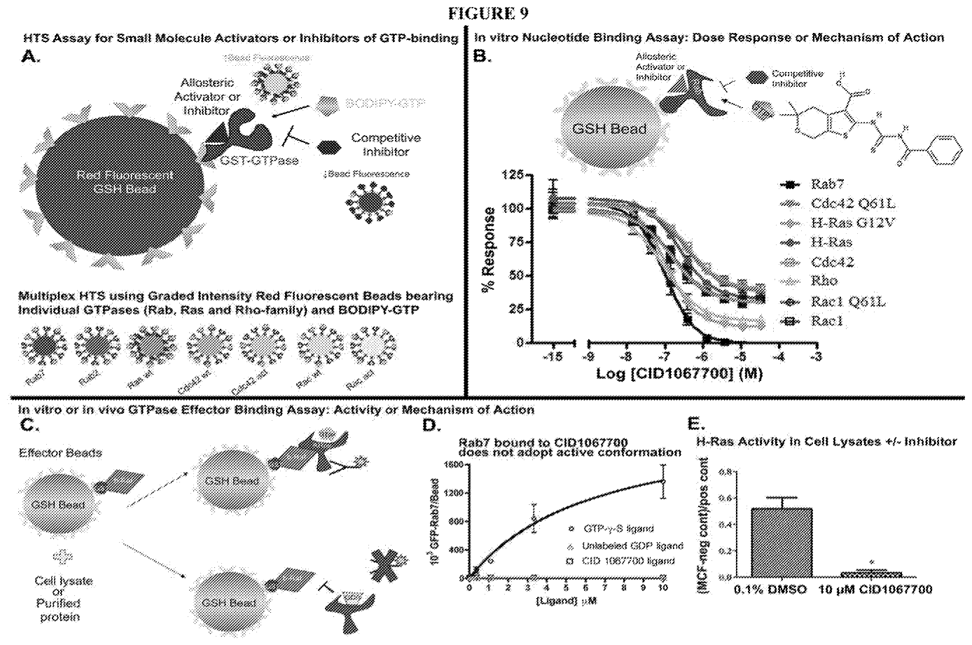

To identify molecules active on and selective for KRas, the inventors took advantage of a multiplex HTS platform (FIG. 10) that was previously described for Rho, Rab, and Hras families, but not KRas³⁵. These studies led to the identification of pan activators, pan inhibitors, selective inhibitors, and repurposed drugs. Assay performance was robust for all of the GTPases based on the Z′ screening reliability statistic⁵⁸. Approximately 1000 compounds including FDA approved drugs were selected and tested in secondary dose-response assays (FIG. 6B leading to the identification of several novel GTPase inhibitors, one evidencing utility in human cancer treatment based on Rac1 and Cdc42 inhibitory activity^{34,42,45-47,67}. One competitive guanine nucleotide binding inhibitor (CID1067700) showed inhibitory activity against H-Ras and H-RasG12V but also functioned as a broad spectrum inhibitor of the Rab and Rho subfamilies (FIG. 10C-E)^{34,53}. A selective inhibitor of the Rho-family protein Cdc42 (cell division control protein 42), that acts as a noncompetitive allosteric inhibitor⁴⁵ and a Rho family selective inhibitor were also identified⁴². The combination of high throughput screening leads and cheminformatic analyses predicted the FDA-approved drug Toradol™ ([R, S] ketorolac) as a Rac1 and/or Cdc42 inhibitor^{46,47,67,68}. Taken together, we have identified new chemical entities selective for class, family, and individual GTPases.

Cell-based assay assays. Inhibitors identified in these earlier studies were evaluated in cells to determine whether GTPase activities were impaired, as quantified using a flow based effector binding G-TRAP assay (FIG. 10C)^{43,44,47}. For example, the assay was able to distinguish individual ketorolac isomers and revealed that the IC₅₀ values for R-ketorolac inhibitory activity against the GTPases (0.5-1 μM) are 2-3 orders of magnitude less than S-ketorolac. The reverse is true for the enantiomer-selective inhibitory activities against cyclooxygenase (COX) enzymes (not shown).

Translation of repurposed drug. The inventors have translated repurposed drugs targeting Rho GTPases as a novel intervention for ovarian cancer⁶⁹⁻⁷¹ with detailed biochemical, cellular, and human data demonstrating that the R-enantiomer of an FDA approved NSAID, [R,S]-ketorolac, possesses a previously unrecognized pharmacologic property as a selective inhibitor of the Ras-related, Rac1 and Cdc42 GTPases with anti-tumor activity^{46,47,67,68}.

In summary, the present invention developed an innovative toolset that includes multiplexing with color-coded microspheres for: a) simultaneous high throughput screening of multiple GTPases to identify regulators of nucleotide binding^{34,42,53,55,59}; b) quantitative analyses of cellular GTPase activities using small volume samples^{43,44}; and c) small molecule mechanism of action studies through real-time kinetic measurements of ligand or effector binding⁵². The inventors have guided production of multiplexed beads from commercial vendors and sourced mutant KRas constructs and cell-lines from NCI Frederick/Leidos. This screening and multi-tiered analysis platform previously identified allosteric, selective inhibitors of Rho-family GTPases with clinical applicability^{53,70} as well as bioactives and repurposed drugs for KRas. This toolkit can be deployed to uncover novel KRas selective compounds, an area that remains relatively underexplored, which should help define the principles of KRas druggability and identify leads for therapeutic development.

One of the active molecules, guanabenz acetate has comparable activity to a recently described KRas inhibitor with low μM affinity¹⁹, but does not appear to limit selectivity to G12D. To our knowledge, this is the first report of an approved drug selective for Ras family GTPases.

All references cited herein are incorporated by reference herein.

### REFERENCES

1. Bos, J. L., Rehmann, H. & Wittinghofer, A. GEFs and GAPs: critical elements in the control of small G proteins. Cell 129, 865-877 (2007).
2. Shaw, R. J. & Cantley, L. C. Ras, PI(3)K and mTOR signalling controls tumour cell growth. Nature 441, 424-430 (2006).
3. Wittinghofer, A. Signal transduction via Ras. Biol Chem 379, 933-937 (1998).
4. Milburn, M. V., et al. Molecular switch for signal transduction: structural differences between active and inactive forms of protooncogenic ras proteins. Science 247, 939-945 (1990).
5. Cox, A. D., Fesik, S. W., Kimmelman, A. C., Luo, J. & Der, C. J. Drugging the undruggable RAS: Mission possible? Nat Rev Drug Discov 13, 828-851 (2014).
6. Fernandez-Medarde, A. & Santos, E. Ras in cancer and developmental diseases. Genes Cancer 2, 344-358 (2011).
7. Stephen A. G., Esposito, D., Bagni, R. K. & McCormick, F. Dragging ras back in the ring. Cancer Cell 25, 272-281 (2014).
8. Barcelo, C., et al. Oncogenic K-ras segregates at spatially distinct plasma membrane signaling platforms according to its phosphorylation status, J Cell Sci 126, 4553-4559 (2013).
9. Bhagatji, P., Leventis, R., Rich, R., Lin, C. J. & Silvius, J. R. Multiple cellular proteins modulate the dynamics of K-ras association with the plasma membrane. Biophys J 99, 3327-3335 (2010).
10. Blazevits, O., et al. Galectin-1 dimers can scaffold Raf-effectors to increase H-ras nanoclustering. Sci Rep 6, 24165 (2016).
11. Muratcioglu, S., et al. GTP-Dependent K-Ras Dimerization. Structure 23, 1325-1335 (2015).
12. Prior, I. A., Muncke, C., Parton, R. G. & Hancock, J. F. Direct visualization of Ras proteins in spatially distinct cell surface microdomains, J Cell Biol 160, 165-170 (2003).
13. Athuluri-Divakar, S. K., et al. A Small Molecule RAS-Mimetic Disrupts RAS Association with Effector Proteins to Block Signaling. Cell 165, 643-655 (2016).
14. Gao, Y., Dickerson, J. B., Guo, F., Zheng, J. & Zheng, Y. Rational design and characterization of a Rac GTPase-specific small molecule inhibitor. Proc Natl Acad Sci USA 101, 7618-7623 (2004).
15. Kauke, M. J., et al. An engineered protein antagonist of K-ras/B-af interaction. Sci Rep 19, 5831 (2017).
16. Niida, A., et al. Investigation of the structural requirements of K-Ras(G12D) selective inhibitory peptide KRpep-2d using alanine scans and cysteine bridging, Bioorg Med Chem Lett 27, 2757-2761 (2017).
17. Shima, F., et al., In silico discovery of small-molecule Ras inhibitors that display antitumor activity by blocking the Ras-effector interaction PNAS 110, 8182-8187 (2013).
18. Spencer-Smith, R., et al. Inhibition of RAS function through targeting an allosteric regulatory site. Nat Chem Biol 13, 62-68 (2017).
19. Welsch, M. E., et al. Multivalent Small-Molecule Pan-RAS Inhibitors. Cell 168, 878-889 e829 (2017).
20. Maurer, T., et al. Small-molecule ligands bind to a distinct pocket in Ras and inhibit SOS-mediated nucleotide exchange activity. Proc Natl Acad Sci USA 109, 5299-5304 (2012).
21. Cox, A. D., Der, C. J., & Philips, M. R. Targeting Ras membrane association: Back to the Future for Anti-Ras Drug Discovery. Clinical cancer research: an official journal of the American Association Cancer Research 15, 1819-1827 (2015).
22. Konstantinopoulos, P. A., Karamouzis, M. V. & Papavassiliou, A. G. Post-translational modifications and regulation of the RAS superfamily of GTPases as anticancer targets. Nat Rev Drug Discov 6, 541-555 (2007).
23. Tothova, Z. & Ebert, B. L. Doubling Down on Mutant RAS Can MEK or Break Leukemia. Cell 168, 749-750 (2017).
24. Adrian, F. J., et al. Allosteric inhibitors of Bcr-abl-dependent cell proliferation. Nat Chem Biol 2, 95-102 (2006).
25. Endicott, J. A., Noble, M. E. & Johnson, L. N. The structural basis for control of eukaryotic protein kinases. Annu Rev Biochem 81, 587-613 (2012).
26. Melnikova, I., Golden J. Targeting protein kinases. Nat Rev Drug Discov 3, 993-994 (2004).
27. Roskoski, R., Jr. A historical overview of protein kinases and their targeted small molecule inhibitors. Pharmacol Res 100, 1-23 (2015).
28. Ruegg, U. T. & Burgess, G. M. Staurosporine, K-562 and UCN-01: potent but non-specific inhibitors of protein kinases, Trends Pharmacol Sci 10, 218-220 (1989).
29. Liu, Y. & Gray, N. S. Rational design of inhibitors that bind to inactive kinase conformations. Nat Chem Biol 2, 358-364 (2006).
30. Niemann, H. H., Knetsch, M. L., Scherer, A., Manstein, D. J. & Kull, K. F. Crystal structure of a dynamin GTPase domain in both nucleotide-free and GDP-bound forms, EMBO J 20, 5813-5821 (2001).
31. Bourne, H. R., Sanders, D. A. & McCormick, F. The GTPase superfamily: conserved structure and molecular mechanism. Nature 349, 117-127 (1991).
32. John, J., et al. Kinetics of Interaction of Nucleotides with Nucleotide-Free H-ras p21. Biochemistry 29, 6058-6065 (1990).
33. Vigil, D., Cherfils, J., Rosman, K. L. & Der, C. J. Ras superfamily GEFs and GAPs: validated and tractable targets for cancer therapy? Nat Rev Cancer 10, 842-857 (2010).
34. Hong, L., et al. A small molecule pan-inhibitor of Ras-superfamily GTPases with high efficacy towards Rab7. in Probe Reports from the NIH Molecular Libraries Program (National Center for Biotechnology Information (US), Bethesda, Md., 2010).
35. Surviladze, Z., et al. Three small molecule pan activator families of Ras-related GTPases. in Probe Reports from the NIH Molecular Libraries Program (National Center for Biotechnology Information (US), Bethesda, Md., 2010).
36. Surviladze, Z., et al. A Potent and Selective Inhibitor of Cdc42 GTPase. in Probe Reports from the NIH Molecular Libraries Program (National Center for Biotechnology Information (US), Bethesda, Md., 2010).
37. Shang, X., et al. Rational design of small molecule inhibitors targeting RhoA subfamily Rho GTPases, Chem Biol 19, 699-710 (2012).
38. Nishikimi, A., et al. Blockade of inflammatory responses by a small molecule inhibitor of Rac activator DOCK2. Chem Biol 19, 488-497 (2012).
39. Shang, X., et al. Small-molecule inhibitors targeting G-protein coupled Rho guanine nucleotide exchange factors. Proc Natl Acad Sci USA 110, 3155-3160 (2013).
40. Zimmermann, G., et al. Small molecule inhibition of the KRAS-PDEδ interaction impairs oncogenic KRAS signalling. Nature 497, 638-642 (2013).
41. Kopra, K., et al. High-Throughput dual screening method for Ras activities and inhibitors. Anal Chem 89, 4508-4516 (2017).
42. Surviladze, Z., et al. Identification of a small GTPase inhibitor using a high-throughput flow cytometry bead-based multiplex assay. J Biomol Screen 15, 10-20 (2010).
43. Buranda, T., et al. Rapid parallel flow cytometry assays of active GTPases using effector beads. Anal Biochem 442, 149-157 (2013).
44. Agola, J. O., et al. Quantitative bead-based flow cytometry for assaying Rab7 GTPase interaction with the Rab-interacting lysosomal protein (RILP) effector protein. Methods Mol Biol 1298, 331-354 (2015).
45. Hong, L., et al. Characterization of a Cdc42 protein inhibitor and its use as a molecular probe. J Biol Chem 288, 8531-8543 (2013).
46. Guo, Y., et al. A Novel Pharmacologic Activity of Ketorolac for Therapeutic Benefit in Ovarian Cancer Patients. Clinical cancer research: an official journal of the American Association for Cancer Research 21, 5064-5072 (2015).
47. Oprea, T. I., et al. Novel Activities of Select NSAID R-Enantiomers against Rac1 and Cdc42 GTPases, PLoS One 10, e0142182 (2015).
48. Palsuledesai, C. C., et al. Activation of Rho family GTPases by small molecules. ACS Chem. Biol. (2018).
49. Hobbs, G. A., Wittinghofer, A. & Der, C. J. Selective Targeting of the KRAS G12C Mutant: Kicking KRAS When It's Down. Cancer Cell 29, 251-253 (2016).
50. Lito, P., Solomon, M., Li, L. S., Hansen, R. & Rosen, N. Allele-specific inhibitors inactivate mutant KRAS G12C by a trapping mechanism. Science 351, 604-608 (2016).
51. Ostrem, J. M., Peters, U., Sos, M. L., Wells, J. A. & Shokat, K. M. K-Ras(G12C) inhibitors allosterically control GTP affinity and effector interactions. Nature 503, 548-531 (2013).
52. Agola, J. O., et al. A Competative Nucleotide Binding inhibitor: In Vitro Characterization of Rab7 GTPase Inhibition. ACS Chem Biol 7, 1095-1108 (2012).
53. Hong, L., et al. A Pan-GTPase Inhibitor as a Molecular Probe. PloS One 10, e0134317 (2015).
54. Schwartz, S. L., et al. Flow Cytometry for Real-Time Measurement of Guanine Nucleotide Binding and Exchange by Ras-like GTPases. Anal Biochem 381, 258-266 (2008).
55. Surviladze, Z., Young, S. & Sklar, L. A. High Throughput Flow Cytometry Bead-based Multiplex Assay for Identification of Rho GTPase Inhibitors. Methods Mol Biol 827, 253-270 (2012).
56. Tessema, M., et al. Glutathione-S-transferase-green fluorescent protein fusion protein reveals slow dissociation from high site density beads and measures free GSH. Cytometry. Part A: the journal of the International Society for Analytical Cytology 69, 326-334 (2006).
57. (website) cancer.gov/research/key-initiatives/ras/screens-assays/model-development/cell-lines.
58. Zhang, J.-H., Chung, T. D. Y. & Oldenburg, K. R. A simple statistical parameter for use in evaluation and validation of high throughput screening assays. J Biomol Screen 4, 67-73 (1999).
59. Glutathione beads and GST fusion proteins. (2011).
60. McEwen, D. P., Gee, K. R., Kang, H. C. & Neubig, R. R. Fluorescent BODIPY-GTP Analogs: Real-Time measurement of nucleotide binding to G proteins Anal Biochem 291, 109-117 (2001).
61. Korlach, J., et al Spontaneous nucleotide exchange in low molecular weight GTPases by fluorescently labeled-phosphate-linked GTP analogs, Proc Natl Acad Sci USA 101, 2800-2805 (2004).
62. Hunter, J. C., et al. Biochemical and structural analysis of common cancer-associated KRAS mutations. Mol Cancer Res 13, 1325-1335 (2015).
63. Schopel, M., et al. Allosteric activation of GDP-bound Ras isoforms by bisphenol derivative plasticisers. Int. J. Mol. Sci. 19, 1133-1147 (2018).
64. Herrmann, C., et al. Sulindac sulfide inhibits Ras signaling. Oncogene 17, 1769-1776 (1998).
65. Wang, W. J. S., Liao, M. & Xia, C. Q. Confidence assessment of an absorption model using limited solubility and permeability data for 21 drugs within a dynamic physiologically-based pharmacokinetic simulator. JABP 3(2015).
66. Bagli, M., et al. Pharmacokinetics of chlorprothixene after single intravenous and oral administration of three galenic preparations. Arzneimittelforschung 46, 247-250 (1996).
67. Guo, Y., et al. R-Ketorolac Targets Cdc42 and Rac1 and Alters Ovarian Cancer Cell Behaviors Critical for Invasion and Metastasis. Molecular cancer therapeutics 14, 2215-2227 (2015).
68. Peretti, A. S., et al. The R-enantiomer of ketorolac delays mammary tumor development in mouse mammary tumor virus-polyoma middle T antigen (MMTV-PyMT) mice. Am J Pathol.
69. Wandinger-Ness, A., et al. Treating or inhibiting ovarian cancer in a patient, comprises administering N-(3-formyl-3-hydroxy-5,5-dimethyl-4,7-dyhydrothieno(2,3-c)pyran-2-yl)carbamoyl)benzamide.
70. Wandinger-Ness, A., et al. Rab7 GTPase inhibitors and related methods of treatment. (2016).
71. Wandinger-Ness, A., Hudson, L. G., Sklar, L., Surviladze, Z. & Oprea, T. Treating ovarian cancer in human patients by measuring expression or activity of rat sarcoma-related C3 botulinum toxin substrate 1 or cell division control protein 42 in sample, and administering composition comprising ketorolac (USA, 2017).

1. A method of identifying a compound as a potential selective agonist, antagonist, or regulator of a protein in a flow cytometer comprising
a. providing in a flow cytometer a multiplex comprising derivatized flow cytometer beads wherein each said derivatized bead is bound to a GST fusion protein comprising a fused protein and a fluorescently labeled binding partner of said fused protein bound thereto, wherein said binding partner emits fluorescent light upon excitation;
b. exposing said flow cytometer bead multiplex from step a to a solution comprising at least one compound of unknown activity; and
c. identifying said at least one compound of said solution as a potential agonist, antagonist, or regulator of said fused protein within said multiplex if said compound displaces or impacts the binding of said fluorescently labeled binding partner as evidenced by a reduction or increase in the fluorescent light being emitted.

2. The method according to claim 1 wherein said solution comprises a library of compounds.

3. The method according to claim 2 wherein said compounds are organic small molecules.

4. The method according to claim 1 wherein said compound identified as a potential agonist, antagonist, or regulator of said fused protein in said assay (first assay) is subjected to a second assay comprising derivatized flow cytometer beads comprising fused proteins to determine the activity of said compound as an agonist, antagonist, or regulator of fused proteins, wherein said second assay comprises a multiplex of derivatized flow cytometer beads wherein each of said derivatized beads is bound to a GST fusion protein comprising at least one fused protein and a fluorescently labeled binding partner of said fused protein bound thereto, wherein said derivatized beads of said second assay comprise fused proteins other than the fused proteins in said first assay, wherein said flow cytometer bead multiplex in said second assay is exposed to a solution comprising at least one compound identified as an agonist, antagonist, or regulator of fused protein in said first assay and identifying said compound from said first assay as a potential agonist, antagonist, or regulator of said fused protein within said multiplex of said second assay if said compound displaces or impacts the binding of said fluorescently labeled binding partner in said second assay as evidenced by a reduction or increase in the fluorescent light being emitted compared to a standard.

5. The method according to any of claims 1-4 wherein said fused protein is a GTPase.

6. The method according to any of claims 1-4 wherein said fused protein is a GST-GTPase.

7. The method according to claim 5 wherein said GTPase is a Rab, Rac, Rho, Cdc42, Ran, or Ras GTPase.

8. The method of any of claims 5-7 wherein said binding partner is GTP.

9. The method according to any of claims 5-8 wherein said GTPase is a mutant KRas GTPase.

10. A method of identifying a compound or portion of a compound as a binding partner of a protein in a flow cytometer comprising:
d. providing in a flow cytometer derivatized flow cytometer beads contained within a multiplex wherein said derivatized beads are each bound to a GST fusion protein comprising GST and a fused protein;
e. exposing said flow cytometer bead multiplex from step a to a solution comprising at least one fluorescently labeled compound having the potential for binding to said fused protein; and
f. identifying a compound of said solution as a potential binding partner of said fused protein if said compound binds to said fused protein as evidenced by an increase in the fluorescent light being emitted from said fused protein.

11. The method according to claim 10 wherein said compound is a protein or polynucleotide.

12. The method according to claim 10 or 11 wherein said solution comprises a series of fluorescently labeled polypeptides or polynucleotides of varying lengths and sequences obtained from a protein or polynucleotide known to be a binding partner of said fused protein.

13. The method according to claim 11 or 12 wherein said compound which binds to said fused protein is further identified by sequencing.

14. The method according to any of claims 1-13 wherein said fused protein requires the presence of another molecule in order for said binding partner to bind to said based protein.

15. A method of identifying a compound or portion of a compound as a binding partner of a protein in a flow cytometer comprising
f. providing in a flow cytometer a population of derivatized flow cytometer beads wherein each of said derivatized beads is bound to a GST fusion protein comprising GST and a fused protein which is fluorescently labeled;
g. exposing said flow cytometer beads from step a to a solution comprising at least one compound having the potential for binding to said fused protein;
h. identifying said compound or a region of said compound as a potential binding partner of said fused protein if said compound binds to said fused protein as evidenced by a decrease in the fluorescent light being emitted from said fused protein;
i. determining the selectivity of said compound identified in step c with respect to individual GTPases by exposing a multiplex of individual fluorescent flow cytometer beads comprising individual GTPases to a solution comprising said compound identified in step c and comparing the binding of said compound with said individual GTPases on said individual fluorescent flow cytometer beads; and
j. determining the selectivity of said compound identified in step c with respect to individual KRas mutants by exposing a multiplex of fluorescent flow cytometer beads comprising individual KRas mutant GTPases to a solution comprising said compound identified in step c and comparing the binding of said compound with said individual KRas mutant GTPases on said fluorescent flow cytometer beads, wherein the selectivity of said compound with respect to KRas mutants and other GTPases is determined by comparing the activities of said compound on said multiplexes comprising both KRas mutant and Ras GTPases with a standard.

16. The method according to claim 15 wherein said solution comprises a series of polypeptides or polynucleotides of varying lengths and sequences obtained from a protein or polynucleotide known to be a binding partner of said fused protein.

17. The method according to claim 16 wherein said compound which binds to said fused protein is further identified by sequencing.

18. The method according to any of claims 1-17 wherein said flow cytometer is a high throughput flow cytometer.

19. The method according to any of claims 10-18 wherein said fused protein is a GTPase.

20. The method according to claim 19 wherein said GTPase is a Rab, Rho, Ran or Ras GTPase.

21. The method according to claim 19 or 20 wherein said GTPase is a Rac or Cdc42 GTPase.

22. The method of any of claims 15-21 wherein said binding partner of GTPase is GTP.

23. The method according to any of claims 15-20 wherein said GTPase is a mutant KRas GTPase.

24. A method according to any of claims 1-23 wherein said flow cytometer is a high throughput flow cytometer.

25. A pharmaceutical composition comprising an effective amount of a GTPase modulator in combination with a pharmaceutically acceptable carrier, additive or excipient

26. The composition according to claim 25 wherein said modulator is an inhibitor of GTPase.

27. The composition according to claim 25 wherein said modulator is an agonist of GTPase.

28. The composition according to claim 25 wherein said modulator is a regulator of GTPase.

29. The composition according to either of claim 25 or 26 wherein said modulator is an inhibitor of Ras GTPase.

30. The composition according to either of claim 25 or 27 wherein said modulator is an agonist of Ras GTPase.

31. The composition according to claim 25 or 26 wherein said modulator is a pan inhibitor of GTPase.

32. The composition according to claim 25 or 27 wherein said modulator is a pan agonist of GTPase.

33. The composition according to claim 25 or 26 wherein said modulator is a selective inhibitor of Ras GTPase.

34. The composition according to claim 25 or 27 wherein said modulator is a selective agonist of Ras GTPase.

35. The composition according to claim 25 wherein said modulator is a mixed activity modulator.

36. The composition according to claim 25 wherein said modulator is a modulator with different activities within a family of GTPases.

37. The composition according to claim 36 wherein said family of GTPases is the Ras GTPases.

38. The composition according to claim 25 wherein said GTPase modulator is selected from the group consisting of Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid, Iopanic acid, Menindione, Iopanic acid, Istradefylline, PR-619, N6022, Diffractaic acid, IPA 3, Fisetin, Folic acid, GSK 3787, Guanabenz acetate, Chlorprothixene hydrochloride, NSC 663284, Ipsapirone, GF109203X, Beta Lapachone, SF1670, Darapladib (SB480848), PD 198306, Pimethixene Maleate, Oxyquinoline Hemisulfate, a pharmaceutically acceptable salt or alternative salt thereof, a stereoisomer thereof or a mixtures thereof.

39. The composition according to any of claims 25-38 further comprising an additional bioactive agent.

40. A modulator of GTPase which is selected from the group consisting of Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid, Iopanic acid, Menindione, Iopanic acid, Istradefylline. PR-619, N6022, Diffractaic acid, IPA 3, Fisetin, Folic acid, GSK 3787, Guanabenz acetate, Chlorprothixene hydrochloride, NSC 663284, Ipsapirone, GF109203X, Beta Lapachone, SF1670, Darapladib (SB480848), PD 198306, Pimethixene Maleate, Oxyquinoline Hemisulfate, a pharmaceutically acceptable salt or alternative salt thereof, a stereoisomer thereof or a mixtures thereof.

41. A method of treating a disease state or condition which is mediated through a GTPase in a patient in need comprising administering to said patient an effective amount of a composition selected from the group consisting of Salsalate, Tolfenamic acid, Dexibuprofen, Mefenamic Acid, Ibuprofen, S-(+)-Ibuprofen, Meclofenamic acid sodium salt monohydrate, (R)-Naproxen sodium salt, Naproxen, Flufenamic Acid, Flurbiprofen, Pheninidione, Dioxybenzone, A-7 hydrochloride, Usnic acid, Iopanic acid, Menindione, Iopanic acid, Istradefylline, PR-619, N6022, Diffractaic acid, IPA 3, Fisetin, Folic acid, GSK 3787, Guanabenz acetate, Chlorprothixene hydrochloride, NSC 663284, Ipsapirone, GF109203X Beta Lapachone, SF1670, Darapladib (SB480848), PD 198306, Pimethixene Maleate, Oxyquinoline Hemisulfate, a pharmaceutically acceptable salt or alternative salt thereof, a stereoisomer thereof or a mixtures thereof.

42. The method according to claim 41 wherein said disease state or condition is cancer, a histiocyte disorder, Noonan syndrome (NS), Noonan syndrome with multiple lentigines, Leopard syndrome, cardiofacio-cutaneous syndrome, neurofibromatosis type I (NF1) and secondary effects of neurofibromatosis type I, Legius syndrome, Costello syndrome (CS), capillary malformation-arteriovenous malformation syndrome (CFC syndrome), congenital myopathy with excess of muscle spindles (CMEMS), congenital heart disease, hereditary gingival fibromatosis type 1 or hypertrophic cardiomyopathy (HCM).

43. The method according to claim 42 wherein said disease state or condition is cancer.

44. The method according to claim 42 or 43 wherein said cancer is a naïve, recurrent, drug resistant or metastatic cancer.

45. The method according to claim 43 or 44 wherein said treatment further comprising co-administering an additional anticancer agent.

46. The method according to any of claims 43-45 wherein said cancer is selected from the group consisting of carcinomas (e.g., squamous-cell carcinomas, basal cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, ovary, pancreas, prostate, and stomach; hematologic cancers, including leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; blastomas, including glioblastoma and medulloblastoma (brain tumors), hepatoblastoma (liver tumor), nephroblastoma (kidney tumor), neuroblastoma (neural tumor), osteoblastoma (bone tumor) and retinoblastoma (retinal tumor in the eye), tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line (germ cell) tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas.

47. The method according to any of claims 43-45 wherein said cancer is thyroid cancer, salivary duct carcinoma, epithelial-myoepithelial carcinoma, kidney cancer, astrocytoma, and melanoma.

48. The method according to claim 43 or 44 wherein said cancer is choriocarcinoma, testicular choriocarcinoma, non-seminomatous germ cell testicular cancer, placental cancer (trophoblastic tumor) or embryonal cancer.

49. The method according to any of claims 45-48 wherein said additional anticancer agent is selected from the group consisting of everolimus, trabectedin, abraxane, TLK 286, AV-299, DN 101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR_{I} KRX-0402, lucanthone, LY 317615, neuradiab, vitespan, Rta 744, Sdx 102, talampanel, atraseman, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, CHIR-258); 3-[5-(methylsulfonylpiperadinemethyl)-indolylj-quinolone, vatalanib, AG-013736, AVE-0005, the acetate salt of [D-Ser(But)6, Azgly10](pyro-Glu-His-Trp-Ser-Tyr-D-Ser(But)-Leu-Arg-Pro-Azgly-NH₂ acetate [C₅₉H₈₄N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilumumab, vemurafenib among others among others, including immunotherapy agents such as IDO inhibitors (an inhibitor of indoleamine 2,3-dioxygenase (IDO) pathway) such as Indoximod (NLG-8187), Navoximod (GDC-0919) and NLG PDL1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab, tremelimumab and mixtures thereof.
